(19) United States
(12) Patent Application Publication
(10) Pub. No.: US 2025/0195070 A1
(43) Pub. Date: Jun. 19, 2025

(54) END TOOL OF SURGICAL INSTRUMENT, CARTRIDGE, SURGICAL INSTRUMENT, AND METHOD FOR DRIVING SURGICAL INSTRUMENT

(71) Applicant: LIVSMED INC., Seongnam-si (KR)

(72) Inventors: Jaeyeong LEE, Seongnam-si (KR); Jung Joo LEE, Seongnam-si (KR); Heejin KIM, Seongnam-si (KR); Dongkyu JANG, Seongnam-si (KR)

(21) Appl. No.: 19/073,000

(22) Filed: Mar. 6, 2025

Related U.S. Application Data
(63) Continuation of application No. PCT/KR2023/013467, filed on Sep. 7, 2023.

(30) Foreign Application Priority Data
Sep. 7, 2022 (KR) 10-2022-0113836
Oct. 7, 2022 (KR) 10-2022-0129114

(51) Int. Cl. A61B 17/072; A61B 17/00

(52) U.S. Cl. CPC A61B 17/07207; A61B 2017/00407; A61B 2017/00477; A61B 2017/00862; A61B 2017/07271; A61B 2017/07285

(57) ABSTRACT

Provided is a surgical instrument including an end tool that includes a jaw having a first jaw and a second jaw formed to face the first jaw, a first jaw pulley coupled to the jaw and formed to be rotatable around a first shaft, an opposing pulley formed to be rotatable around a shaft substantially the same as or parallel to the first shaft and formed to be spaced apart from the first jaw pulley by a certain extent, and a staple drive assembly including one or more staple pulleys at least partially formed between the first jaw pulley or the opposing pulley, a cartridge that includes a reciprocating assembly connected to the staple drive assembly and configured to move linearly in response to a rotational movement of the one or more staple pulleys, and an operation member that may be in contact with the reciprocating assembly and formed to move in one direction by the reciprocating assembly when the reciprocating assembly may move in the one direction, and a retraction wire connected to the operation member and formed to pull the operation member toward a proximal end of the jaw.

### CROSS-REFERENCE TO RELATED APPLICATION

This application is a continuation application of PCT International Application No. PCT/KR2023/013467, filed on Sep. 7, 2023, and claims priority to Korean Application No. 10-2022-0113836, filed on Sep. 7, 2022, and Korean Application No. 10-2022-0129114, filed on Oct. 7, 2022, in the Korean Intellectual Property Office, the entire disclosures of which are incorporated herein by reference.

### TECHNICAL FIELD

The present disclosure relates to an end tool and a cartridge of a surgical instrument, the surgical instrument, and a method for driving the surgical instrument.

### BACKGROUND ART

In recent years, laparoscopic surgery has been actively used to reduce postoperative recovery time and complications through small incisions. The laparoscopic surgery is a surgical method in which a plurality of small holes are drilled in the abdomen of a patient and the inside of the abdominal cavity is observed through these holes, and is widely used in general surgery and the like.

In performing such laparoscopic surgery, various instruments are being used. For example, suturing instruments that are inserted into the body to suture the surgical site within the abdominal cavity are used, and a surgical stapler that uses medical staples to suture the surgical site is used as the suturing instrument.

In general, a surgical stapler is a medical instrument that is often used for cutting and anastomosis of an organ in abdominal and thoracic surgery. The surgical stapler includes an open stapler used in thoracotomy and laparotomy and an endo stapler used in thoracoscopic surgery and celioscopic surgery.

The surgical stapler has advantages of not only shortening operation time since cutting of a surgical site and anastomosis of an organ are simultaneously performed, but also accurately suturing the surgical site. In addition, the surgical stapler has advantages of a faster recovery and a smaller scar than those when tissue is cut and sutured using a surgical stapling thread, and thus has been widely used in modern surgical operations. In particular, the surgical stapler has been widely used in cancer surgery to cut cancer tissue and suture a cut site.

### DESCRIPTION

### Technical Problem

The present disclosure is directed to providing a surgical instrument that may be mounted on a robotic arm or operated manually for use in laparoscopic surgery or various other surgeries, and includes an end tool that is rotatable in two or more directions and operates in a way that intuitively matches a motion of a manipulation part.

### Technical Solution

An embodiment of the present disclosure discloses a surgical instrument including an end tool that includes a jaw having a first jaw and a second jaw formed to face the first jaw, a first jaw pulley coupled to the jaw and formed to be rotatable around a first shaft, an opposing pulley formed to be rotatable around a shaft substantially the same as or parallel to the first shaft and formed to be spaced apart from the first jaw pulley by a certain extent, and a staple drive assembly including one or more staple pulleys at least partially formed between the first jaw pulley or the opposing pulley, a cartridge that includes a reciprocating assembly connected to the staple drive assembly and configured to move linearly in response to a rotational movement of the one or more staple pulleys, and an operation member that may be in contact with the reciprocating assembly and formed to move in one direction by the reciprocating assembly when the reciprocating assembly may move in the one direction, and a retraction wire connected to the operation member and formed to pull the operation member toward a proximal end of the jaw.

### Advantageous Effects

An end tool, a cartridge, a surgical instrument, and a method for driving the surgical instrument according to the present disclosure can provide the advantages of improving user convenience as well as enhancing the accuracy, reliability, and speed of surgeries.

### DESCRIPTION OF DRAWINGS

Panel (a) of FIG. 1 is a conceptual diagram of a pitch motion of a conventional surgical instrument, and panel (b) of FIG. 1 is a conceptual diagram of a yaw motion.

Panel (c) of FIG. 1 is a conceptual diagram of a pitch motion of another conventional surgical instrument, and panel (d) of FIG. 1 is a conceptual diagram of a yaw motion.

Panel (e) of FIG. 1 is a conceptual diagram of a pitch motion of a surgical instrument according to the present disclosure, and panel (f) of FIG. 1 is a conceptual diagram of a yaw motion.

FIG. 2 is a perspective view illustrating a surgical instrument according to a first embodiment of the present disclosure.

FIG. 3 is a side view of the surgical instrument of FIG. 2.

FIGS. 4 and 5 are perspective views illustrating an end tool of the surgical instrument of FIG. 2.

FIG. 6 is a perspective view illustrating an end tool hub of the end tool of the surgical instrument of FIG. 2.

FIGS. 7 to 10 are plan views illustrating the end tool of the surgical instrument of FIG. 4.

FIG. 11 is a side view illustrating the end tool of the surgical instrument of FIG. 4.

FIGS. 12 to 15 are exploded perspective views of the end tool of the surgical instrument of FIG. 4.

FIG. 16 is a side view illustrating a second jaw of the surgical instrument of FIG. 4.

FIG. 17 is a cross-sectional view illustrating the second jaw of the surgical instrument of FIG. 4.

FIG. 18 is a side view illustrating a first jaw of the surgical instrument of FIG. 4.

FIG. 19 is a cross-sectional view illustrating the first jaw of the surgical instrument of FIG. 4.

FIG. 20 is a cross-sectional view illustrating an open state of the first and second jaws of the surgical instrument of FIG. 4.

FIG. 21 is a cross-sectional view illustrating a closed state of the first and second jaws of the surgical instrument of FIG. 4.

FIGS. 22 and 23 are exploded perspective views illustrating a staple drive assembly of the surgical instrument of FIG. 4.

FIGS. 24 and 25 are side views illustrating an operation of the staple drive assembly of the surgical instrument of FIG. 4.

FIGS. 26 and 27 are perspective views illustrating operations of the staple drive assembly of the surgical instrument of FIG. 4.

FIG. 28 is a perspective view illustrating the first jaw and a cartridge of the surgical instrument of FIG. 2.

FIG. 29 is an exploded perspective view illustrating the cartridge of FIG. 28, and FIG. 30 is a side cross-sectional view illustrating the cartridge of FIG. 28.

FIGS. 31 and 32 are perspective views of an operation member of the cartridge of FIG. 28 viewed from one direction.

FIGS. 33 and 34 are perspective views of the operation member of the cartridge of FIG. 28 viewed from another direction.

FIGS. 35 and 36 are perspective cross-sectional views illustrating a stapling-related structure of the end tool of the surgical instrument of FIG. 2.

FIGS. 37 to 40 are perspective views illustrating a ratchet drive operation of the end tool of FIG. 35.

FIGS. 41 and 42 are plan views illustrating the ratchet drive operation of the end tool of FIG. 35.

FIG. 43 is a perspective view illustrating an entire ratchet drive operation of the end tool of FIG. 35.

FIGS. 44 and 45 are cross-sectional views illustrating the entire stapling motion of the end tool of FIG. 35.

FIG. 46 is a view illustrating a process in which the operation member is coupled to the second jaw.

FIG. 47 is a plan view illustrating a backward movement of the operation member of the end tool of FIG. 35.

FIGS. 48 to 51 are plan views illustrating an actuation motion of the end tool of the surgical instrument of FIG. 2.

FIGS. 52 to 55 are plan views illustrating an actuation motion of the end tool of the surgical instrument of FIG. 2.

FIGS. 56 and 57 are plan views illustrating a stapling motion of the end tool of the surgical instrument of FIG. 2.

FIGS. 58 and 59 are plan views illustrating a staple motion of the end tool of the surgical instrument of FIG. 2.

FIGS. 60 to 63 are perspective views illustrating a pitch motion of the surgical instrument of FIG. 2.

FIGS. 64 to 67 are perspective views illustrating a yaw motion of the surgical instrument of FIG. 2.

FIGS. 68 to 71 are perspective views illustrating a state in which the end tool of the surgical instrument of FIG. 2 is pitch-rotated and yaw-rotated.

FIG. 72 is a perspective view illustrating a first jaw of an end tool and a cartridge of a surgical instrument according to a second embodiment of the present disclosure.

FIG. 73 is a plan view of an operation member of the cartridge of FIG. 72 viewed from one direction.

FIGS. 74 and 75 are schematic perspective views of the cartridge of FIG. 72.

FIG. 76 is a partial perspective view of the first jaw of the cartridge of FIG. 72.

FIG. 77 is a plan view of the cartridge of FIG. 72 viewed from one direction.

FIG. 78 is a plan view of the cartridge of FIG. 72 viewed from another direction.

FIG. 79 is a view for describing a backward-movement prevention operation of the operation member of FIG. 72.

FIG. 80 is a view for describing a backward-movement prevention release and backward movement of the operation member of FIG. 72.

FIGS. 81 and 82 are perspective views illustrating an end tool of a surgical instrument according to a third embodiment of the present disclosure.

FIGS. 83 to 86 are plan views illustrating the end tool of the surgical instrument of FIG. 81.

FIG. 87 is a side view illustrating the end tool of the surgical instrument of FIG. 81.

FIGS. 88 to 90 are exploded perspective views of the end tool of the surgical instrument of FIG. 81.

FIG. 91 is a perspective view illustrating a first jaw and a cartridge of the surgical instrument of FIG. 81.

FIG. 92 is an exploded perspective view illustrating the cartridge of FIG. 91, and

FIG. 93 is a side cross-sectional view illustrating the cartridge of FIG. 91.

FIGS. 94 and 95 are perspective views of an operation member of the cartridge of FIG. 91 viewed from one direction.

FIGS. 96 and 97 are plan views of the operation member of the cartridge of FIG. 91 viewed from one direction.

FIGS. 98 and 99 are plan views of the cartridge of FIG. 91 viewed from one direction and another direction different from the one direction, respectively.

FIG. 100 is a plan view illustrating a backward-movement prevention operation of the operation member of the end tool of FIG. 91.

FIG. 101 is a plan view illustrating a backward movement of the operation member of the end tool of FIG. 91.

FIGS. 102 and 103 are perspective views illustrating an end tool of a surgical instrument according to a fourth embodiment of the present disclosure.

FIGS. 104 and 105 are plan views illustrating the end tool of the surgical instrument of FIG. 102.

FIG. 106 is a side view illustrating the end tool of the surgical instrument of FIG. 102.

FIGS. 107 and 108 are exploded perspective views of the end tool of the surgical instrument of FIG. 102.

FIGS. 109 and 110 are exploded perspective views of a staple pulley assembly of the surgical instrument of FIG. 102.

FIG. 111 is a side view illustrating operating states of a staple pulley in the end tool of FIG. 102.

FIGS. 112 and 113 are plan views illustrating opening and closing motions of the end tool of the surgical instrument of FIG. 102.

FIG. 114 is a perspective view illustrating a process in which the end tool of the surgical instrument of FIG. 102 is switched from a deactivated state to an activated state.

FIG. 115 is a perspective view illustrating a first jaw and a cartridge of the surgical instrument of FIG. 102.

FIG. 116 is a plan view illustrating a backward movement of an operation member of the end tool of FIG. 102.

### BEST MODE

An embodiment of the present disclosure discloses a surgical instrument including an end tool that includes a jaw having a first jaw and a second jaw formed to face the first jaw, a first jaw pulley coupled to the jaw and formed to be rotatable around a first shaft, an opposing pulley formed to be rotatable around a shaft substantially the same as or parallel to the first shaft and formed to be spaced apart from the first jaw pulley by a certain extent, and a staple drive assembly including one or more staple pulleys at least partially formed between the first jaw pulley or the opposing pulley, a cartridge that includes a reciprocating assembly connected to the staple drive assembly and configured to move linearly in response to a rotational movement of the one or more staple pulleys, and an operation member that may be in contact with the reciprocating assembly and formed to move in one direction by the reciprocating assembly when the reciprocating assembly may move in the one direction, and a retraction wire connected to the operation member and formed to pull the operation member toward a proximal end of the jaw.

In an embodiment, the opposing pulley may be a pulley for the retraction wire, with which the retraction wire comes into contact.

In an embodiment, the opposing pulley may be a second jaw pulley coupled to the second jaw.

In an embodiment, when the one or more staple pulleys rotate, the reciprocating assembly connected to the staple drive assembly may move toward a distal end or a proximal end of the cartridge.

In an embodiment, when the one or more staple pulleys alternately rotate in a clockwise direction and a counterclockwise direction, the reciprocating assembly connected to the staple drive assembly may alternately move toward the distal end and the proximal end of the cartridge.

In an embodiment, when the reciprocating assembly moves toward the distal end of the cartridge, the operation member may move toward the distal end of the cartridge by the reciprocating assembly.

In an embodiment, the staple drive assembly may include a first staple pulley and a second staple pulley that face each other and rotationally move.

In an embodiment, a bidirectional rotational movement of the first staple pulley or the second staple pulley may be converted into a reciprocating linear movement of the reciprocating assembly connected to the staple drive assembly, by the staple drive assembly.

In an embodiment, when the first staple pulley rotates in a first direction among a clockwise direction and a counterclockwise direction, and the second staple pulley rotates in a direction opposite to the first direction among the clockwise and counterclockwise directions, the reciprocating assembly connected to the first and second staple pulleys and the operation member in contact with the reciprocating assembly may move toward a distal end of the cartridge.

In an embodiment, when the first staple pulley rotates in the direction opposite to the first direction among the clockwise and counterclockwise directions, and the second staple pulley rotates in the first direction among the clockwise and counterclockwise directions, the reciprocating assembly connected to the one or more staple pulleys may move toward a proximal end of the end tool, and the operation member may remain stationary with respect to the one direction.

In an embodiment, as the operation member moves in the one direction, a wedge of the operation member may sequentially push and raise a plurality of staples in the cartridge to perform a stapling motion, and simultaneously, a blade formed on one side of the wedge of the operation member may move in the one direction to perform a cutting motion.

In an embodiment, the staple drive assembly may include a link member configured to connect the one or more staple pulleys to the reciprocating assembly.

In an embodiment, the operation member may include a ratchet member having at least one surface on which a ratchet is formed, and the ratchet of the ratchet member may be formed to be in contact with the reciprocating assembly.

In an embodiment, the operation member may move toward a distal end of the cartridge together with the reciprocating assembly only when the reciprocating assembly moves toward the distal end of the cartridge.

In an embodiment, the ratchet member of the operation member and the reciprocating assembly may be separated from each other by the retraction wire.

In an embodiment, the operation member may be formed to be movable toward a proximal end of the end tool by the retraction wire while being spaced apart from the reciprocating assembly.

In an embodiment, the surgical instrument may further include an operation part elastic member disposed on one side of the operation member and configured to apply an elastic force in a direction that brings the ratchet member of the operation member and the reciprocating assembly closer to each other.

In an embodiment, when the retraction wire is pulled, the ratchet member may press the operation part elastic member in a direction in which the operation part elastic member moves away from the reciprocating assembly.

In an embodiment, an elastic member may be disposed between the second jaw and the first jaw.

In a standby state, the second jaw and the first jaw may maintain an open state through the elastic member.

In an embodiment, the elastic member may be disposed to apply an elastic force in a direction in which the second jaw moves away from the first jaw.

In an embodiment, by moving the operation member, one region of the operation member may be disposed inside the second jaw, thereby applying a force that reduces a gap between the second jaw and the first jaw and maintains a closed state of the second jaw and the first jaw.

In an embodiment, the second jaw may be coupled to the first jaw, and may be formed to rotationally move integrally with the first jaw around the first shaft when the first jaw rotates.

In an embodiment, a passageway having a groove shape may be formed in the second jaw in a longitudinal direction of the second jaw, and a clamp of the operation member may be formed to be movable in the passageway.

In an embodiment, a guide part with an opening may be formed in one region of the passageway at a proximal end side to allow the clamp of the operation member to be inserted into the second jaw.

In an embodiment, when the operation member moves from a proximal end toward a distal end of the end tool, one region of the clamp of the operation member may pass through the guide part of the second jaw and may move along the passageway of the second jaw, thereby maintaining a closed state of the second jaw and the first jaw.

In an embodiment, the operation member may be formed to move toward a proximal end of the end tool independently of the reciprocating assembly by pulling the retraction wire.

In an embodiment, the retraction wire and the operation member may be integrally coupled.

In an embodiment, the retraction wire may be formed to be attachable and detachable by being fastened to or unfastened from the operation member.

In an embodiment, a backward-movement prevention member may be formed on one side of the operation member to restrict a backward movement of the operation member caused by the reciprocating assembly when the reciprocating assembly moves backward in a proximal end direction.

In an embodiment, the backward-movement prevention member of the operation member may engage with an engaging part on an inner surface of a space of the end tool, in which the operation member is disposed, to restrict the backward movement of the operation member.

In an embodiment, the engaging part may be formed on an inner side surface of the first jaw and may include one or more protruding regions and one or more grooves.

In an embodiment, the backward-movement prevention member may be formed to perform a rotational movement.

In an embodiment, pulling a backward-movement prevention release wire connected to the backward-movement prevention member may cause the backward-movement prevention member to perform the rotational movement, and the restriction on the backward movement of the operation member by the backward-movement prevention member may be released through the rotational movement of the backward-movement prevention member.

In an embodiment, the surgical instrument may further include one or more staple wires coupled to the one or more staple pulleys and configured to rotate the one or more staple pulleys.

In an embodiment, the surgical instrument may further include a pair of end tool first jaw pitch main pulleys formed to be rotatable around a second shaft that forms a certain angle with the first shaft.

In an embodiment, the end tool may be formed to be yaw-rotatable around the first shaft and simultaneously pitch-rotatable around the second shaft.

In an embodiment, the first jaw pulley, the one or more staple pulleys, and the opposing pulley may be sequentially stacked.

Another embodiment of the present disclosure discloses an end tool of a surgical instrument, the end tool including a first jaw formed to accommodate a cartridge, a second jaw formed to face the first jaw, a first jaw pulley coupled to the first jaw and formed to be rotatable around a first shaft, an opposing pulley formed to be rotatable around a shaft substantially the same as or parallel to the first shaft, and formed to be spaced apart from the first jaw pulley by a certain extent, and a staple drive assembly including one or more staple pulleys at least partially formed between the first jaw pulley or the opposing pulley, a staple wire at least partially in contact with the one or more staple pulleys and configured to transmit, to the one or more staple pulleys, a driving force for rotating the one or more staple pulleys, and a retraction wire connected to an operation member of the cartridge, wherein the staple drive assembly may be connected to a reciprocating assembly of the cartridge and converts a rotational motion of the one or more staple pulleys into a linear motion of the reciprocating assembly.

In an embodiment, the opposing pulley may be a pulley for the retraction wire, with which the retraction wire comes into contact.

In an embodiment, the opposing pulley may be a second jaw pulley coupled to the second jaw.

In an embodiment, The end tool may further include an end tool hub including a first jaw pulley coupling part and a second pulley coupling part, which are formed to face each other, and a guide part configured to connect the first jaw pulley coupling part to the second pulley coupling part, wherein the first jaw pulley may be disposed adjacent to the first jaw pulley coupling part of the end tool hub, the opposing pulley may be disposed adjacent to the second pulley coupling part of the end tool hub, and at least a portion of the staple drive assembly may be formed between the first jaw pulley and the opposing pulley.

In an embodiment, the first shaft may be sequentially inserted through the first jaw pulley coupling part, the first jaw pulley, the one or more staple pulleys, the opposing pulley, and the second pulley coupling part.

In an embodiment, the first jaw pulley, the one or more staple pulleys, and the opposing pulley may be sequentially stacked inside the end tool hub.

In an embodiment, the first jaw pulley, the one or more staple pulleys, and the opposing pulley may be formed to be rotatable independently of each other.

In an embodiment, the end tool may further include a staple auxiliary pulley disposed between the one or more staple pulleys and the guide part.

In an embodiment, the staple wire may be located on a common internal tangent of the one or more staple pulleys and the staple auxiliary pulley, and a rotation angle of the one or more staple pulleys may be increased by the staple auxiliary pulley.

In an embodiment, the staple drive assembly may further include a staple link assembly configured to connect the one or more staple pulleys to the reciprocating assembly.

In an embodiment, the staple link assembly may include a link member coupled to the one or more staple pulleys and the reciprocating assembly.

In an embodiment, when the one or more staple pulleys alternately rotate in a clockwise direction and a counterclockwise direction, the staple link assembly connected to the one or more staple pulleys may alternately move toward a distal end and a proximal end of the end tool.

In an embodiment, the staple drive assembly may include a first staple pulley and a second staple pulley that face each other and rotationally move.

In an embodiment, a bidirectional rotational movement of the first staple pulley or the second staple pulley may be converted into a reciprocating linear movement of the reciprocating assembly connected to the staple drive assembly, by the staple drive assembly.

In an embodiment, when the first staple pulley rotates in a first direction among a clockwise direction and a counterclockwise direction, and the second staple pulley may rotate in a direction opposite to the first direction among the clockwise and counterclockwise directions, the reciprocating assembly connected to the first and second staple pulleys and the operation member in contact with the reciprocating assembly may move toward a distal end of the cartridge.

In an embodiment, when the first staple pulley rotates in the direction opposite to the first direction among the clockwise and counterclockwise directions, and the second staple pulley may rotate in the first direction among the clockwise and counterclockwise directions, the reciprocating assembly connected to the one or more staple pulleys may move in a proximal end direction of the end tool, and the operation member may remain stationary with respect to a direction of the distal end of the cartridge.

In an embodiment, a guide groove may be formed in the first jaw in a longitudinal direction of the first jaw, and the staple link assembly may move along the guide groove.

In an embodiment, the end tool may further include a pair of end tool first jaw pitch main pulleys formed to be rotatable around a second shaft that forms a certain angle with the first shaft.

In an embodiment, the end tool may be formed to be yaw-rotatable around the first shaft and simultaneously pitch-rotatable around the second shaft.

In an embodiment, the end tool may further include a first jaw wire at least partially wound around the first jaw pulley and the pair of end tool first jaw pitch main pulleys.

In an embodiment, an elastic member may be disposed between the second jaw and the first jaw, and in a standby state, the second jaw and the first jaw may maintain an open state through the elastic member.

In an embodiment, the elastic member may be disposed to apply an elastic force in a direction in which the second jaw moves away from the first jaw.

In an embodiment, by moving the operation member, one region of the operation member may be disposed inside the second jaw, thereby applying a force that reduces a gap between the second jaw and the first jaw and maintains a closed state of the second jaw and the first jaw.

In an embodiment, the second jaw may be coupled to the first jaw, and may be formed to rotationally move integrally with the first jaw around the first shaft when the first jaw rotates.

In an embodiment, a passageway having a groove shape may be formed in the second jaw in a longitudinal direction of the second jaw, and a clamp of the operation member may be formed to be movable in the passageway.

In an embodiment, a guide part with an opening may be formed in one region of the passageway at a proximal end side of the second jaw to allow the clamp of the operation member to be inserted into the second jaw.

In an embodiment, when the operation member moves from a proximal end toward a distal end of the end tool, one region of the clamp of the operation member may pass through the guide part of the second jaw and may move along the passageway of the second jaw, thereby maintaining a closed state of the second jaw and the first jaw.

In an embodiment, the operation member may be formed to move toward a proximal end of the end tool independently of the reciprocating assembly by pulling the retraction wire.

In an embodiment, the retraction wire and the operation member may be integrally coupled.

In an embodiment, the retraction wire may be formed to be attachable and detachable by being fastened to or unfastened from the operation member.

In an embodiment, a backward-movement prevention member may be formed on one side of the operation member to restrict a backward movement of the operation member caused by the reciprocating assembly when the reciprocating assembly moves backward in a proximal end direction.

In an embodiment, the backward-movement prevention member of the operation member may engage with an engaging part formed on a corresponding surface of the first jaw to restrict the backward movement of the operation member.

In an embodiment, the backward-movement prevention member may be connected to the backward-movement prevention release wire.

In an embodiment, the restriction on the backward movement of the operation member by the backward-movement prevention member may be released by pulling the backward-movement prevention release wire.

In an embodiment, a ratchet member of the operation member and the reciprocating assembly may be separated from each other by the retraction wire.

In an embodiment, the operation member may be formed to be movable toward a proximal end of the end tool by the retraction wire while being spaced apart from the reciprocating assembly.

In an embodiment, the end tool may further include an operation part elastic member disposed on one side of the operation member and configured to apply an elastic force in a direction that brings the ratchet member of the operation member and the reciprocating assembly closer to each other.

In an embodiment, when the retraction wire is pulled, the ratchet member may press the operation part elastic member in a direction in which the operation part elastic member moves away from the reciprocating assembly.

Another embodiment of the present disclosure discloses an end tool of a surgical instrument, the end tool including a first jaw and a second jaw that are rotatable independently of each other, a first jaw pulley coupled to the first jaw and formed to be rotatable around a first shaft, an opposing pulley formed to be rotatable around a shaft substantially the same as or parallel to the first shaft, one or more staple pulleys formed to be rotatable around the shaft substantially the same as or parallel to the first shaft, and disposed adjacent to the first jaw pulley or the opposing pulley, a retraction wire formed to be pulled at least toward a proximal end of the first jaw, and a staple link assembly connected to the one or more staple pulleys and configured to reciprocate in response to a bidirectional rotation of the one or more staple pulleys.

In an embodiment, the opposing pulley may be a pulley for the retraction wire, with which the retraction wire comes into contact.

In an embodiment, the opposing pulley may be a second jaw pulley coupled to the second jaw.

In an embodiment, the staple link assembly may be coupled to a reciprocating assembly of a cartridge accommodated in the first jaw and may reciprocate the reciprocating assembly.

In an embodiment, the staple link assembly may move toward a distal end or a proximal end of the end tool according to a rotation direction of the one or more staple pulleys.

In an embodiment, a protrusion may be formed on one side of the one or more staple pulleys and the staple link assembly, a hole may be formed on another side of the one or more staple pulleys and the staple link assembly, and the protrusion may be axially coupled to the hole.

In an embodiment, a protruding member may be formed on the one or more staple pulleys, a slot may be formed in the staple link assembly, and when the one or more staple pulleys rotate, the protruding member may move in the slot while coming into contact with the slot.

In an embodiment, the one or more staple pulleys may include a first staple pulley and a second staple pulley that face each other and rotate.

In an embodiment, a bidirectional rotational movement of the first staple pulley or the second staple pulley may be converted into a reciprocating linear movement of the staple link assembly connected to the first staple pulley or the second staple pulley.

In an embodiment, when the first staple pulley rotates in a first direction among a clockwise direction and a counterclockwise direction, and the second staple pulley rotates in a direction opposite to the first direction among the clockwise and counterclockwise directions, a reciprocating assembly, which is accommodated in the first jaw, and an operation member of a cartridge move toward a distal end of the end tool through the staple link assembly connected to the first and second staple pulleys.

In an embodiment, when the first staple pulley rotates in the direction opposite to the first direction among the clockwise and counterclockwise directions, and the second staple pulley rotates in the first direction among the clockwise and counterclockwise directions, the reciprocating assembly connected to the one or more staple pulleys may move toward a proximal end of the end tool, and the operation member may remain stationary with respect to a direction of the distal end of the end tool.

In an embodiment, the operation member may be formed to be movable toward a proximal end of the end tool by the retraction wire while being spaced apart from the reciprocating assembly by the retraction wire.

In an embodiment, the end tool may further include an operation part elastic member disposed on one side of the operation member and configured to apply an elastic force in a direction that brings a ratchet member of the operation member and the reciprocating assembly closer to each other.

In an embodiment, when the retraction wire is pulled, the ratchet member may press the operation part elastic member in a direction in which the operation part elastic member moves away from the reciprocating assembly.

In an embodiment, a guide groove may be formed in the first jaw in a longitudinal direction of the first jaw, and the staple link assembly may move along the guide groove.

In an embodiment, the end tool may include a pair of end tool first jaw pitch main pulleys formed to be rotatable around a second shaft that forms a certain angle with the first shaft.

In an embodiment, when the first jaw pulley rotates around the second shaft, the one or more staple pulleys may rotate together with the first jaw pulley.

In an embodiment, when the first jaw pulley rotates around the first shaft, the one or more staple pulleys may rotate together with the first jaw pulley.

In an embodiment, while the one or more staple pulleys rotate around the first shaft by the staple wire, the first jaw pulley may not rotate.

In an embodiment, an elastic member may be disposed between the second jaw and the first jaw, and in a standby state, the second jaw and the first jaw may maintain an open state through the elastic member.

In an embodiment, the elastic member may be disposed to apply an elastic force in a direction in which the second jaw moves away from the first jaw.

In an embodiment, by moving an operation member, which may be disposed inside the first jaw and receives a driving force from the staple link assembly, one region of the operation member may be disposed inside the second jaw, thereby applying a force that reduces a gap between the second jaw and the first jaw and maintains a closed state of the second jaw and the first jaw.

In an embodiment, the second jaw may be coupled to the first jaw, and may be formed to rotationally move integrally with the first jaw around the first shaft when the first jaw rotates.

In an embodiment, a passageway having a groove shape may be formed in the second jaw in a longitudinal direction of the second jaw, and a clamp of the operation member may be formed to be movable in the passageway.

In an embodiment, a guide part with an opening may be formed in one region of the second jaw at a proximal end side of the end tool to allow the clamp of the operation member to be inserted into the second jaw.

In an embodiment, when the operation member moves from a proximal end toward a distal end of the end tool, one region of the clamp of the operation member may pass through the guide part of the second jaw and may move along the passageway of the second jaw, thereby maintaining a closed state of the second jaw and the first jaw.

In an embodiment, an operation member of a cartridge disposed inside the first jaw may be formed to move toward a proximal end of the end tool independently of the staple link assembly by pulling the retraction wire connected to the opposing pulley.

In an embodiment, a backward-movement prevention member may be formed on one side of the operation member so that a backward movement of the operation member toward a proximal end is restricted by the staple link assembly.

In an embodiment, the backward-movement prevention member may be connected to a backward-movement prevention release wire.

In an embodiment, a cartridge accommodation part in which a cartridge is accommodated may be formed in the first jaw, and an anvil with which a staple of the cartridge is in contact may be formed in the second jaw.

In an embodiment, the end tool may further include a first jaw wire at least partially wound around the first jaw pulley, and one or more staple wires at least partially wound around the one or more staple pulleys.

Another embodiment of the present disclosure discloses an end tool of a surgical instrument, the end tool including a first jaw and a second jaw that are rotatable independently of each other, a first jaw pulley coupled to the first jaw and formed to be rotatable around a first shaft, a first jaw wire at least partially wound around the first jaw pulley, an opposing pulley formed to be rotatable around the first shaft, a retraction wire formed to be pulled at least toward a proximal end of the first jaw, a pair of end tool first jaw pitch main pulleys formed on one side of the first jaw pulley, and formed to be rotatable around a second shaft that forms a certain angle with the first shaft, one or more staple pulleys formed to be rotatable around the first shaft and disposed between the first jaw pulley and the opposing pulley, a staple link assembly connected to the one or more staple pulleys and configured to reciprocate in response to a bidirectional rotation of the one or more staple pulleys, and one or more staple wires at least partially in contact with the one or more staple pulleys and configured to transmit, to the one or more staple pulleys, a driving force for rotating the one or more staple pulleys.

In an embodiment, the opposing pulley may be a pulley for the retraction wire, with which the retraction wire comes into contact.

In an embodiment, the opposing pulley may be a second jaw pulley coupled to the second jaw.

In an embodiment, a bidirectional rotational movement of the one or more staple pulleys may be converted into a reciprocating linear movement of the staple link assembly.

In an embodiment, the staple link assembly may be coupled to a reciprocating assembly of a cartridge accommodated in the first jaw, and a rotational motion of the one or more staple pulleys may be transmitted to an operation member of the cartridge via the staple link assembly and the reciprocating assembly.

In an embodiment, a bidirectional rotational movement of the one or more staple pulleys may be converted into a reciprocating linear movement of the reciprocating assembly, which is connected to the staple link assembly, by the staple link assembly.

In an embodiment, a ratchet member of the operation member and the reciprocating assembly may be separated from each other by the retraction wire.

In an embodiment, the operation member may be formed to be movable toward a proximal end of the end tool by the retraction wire while being spaced apart from the reciprocating assembly.

In an embodiment, the end tool may further include an operation part elastic member disposed on one side of the operation member and configured to apply an elastic force in a direction that brings the ratchet member of the operation member and the reciprocating assembly closer to each other.

In an embodiment, when the retraction wire is pulled, the ratchet member may press the operation part elastic member in a direction in which the operation part elastic member moves away from the reciprocating assembly.

In an embodiment, when the one or more staple pulleys alternately rotate in a clockwise direction and a counterclockwise direction, the staple link assembly connected to the one or more staple pulleys may alternately move toward a distal end and a proximal end of the end tool.

In an embodiment, the one or more staple pulleys may include a first staple pulley and a second staple pulley that face each other and rotate.

In an embodiment, a guide groove may be formed in the first jaw in a longitudinal direction of the first jaw, and the staple link assembly may move along the guide groove.

In an embodiment, a guide groove may be formed in an anvil of the second jaw in a longitudinal direction of the second jaw, and a clamp of a cartridge accommodated in the first jaw may be formed to be movable along the guide groove.

In an embodiment, coupling grooves through which the clamp may be inserted into or withdrawn from the anvil of the second jaw are formed at both end portions of the guide groove of the anvil.

In an embodiment, the end tool may further include a pair of end tool first jaw pitch main pulleys formed on one side of the first jaw pulley, and formed to be rotatable around a second shaft that forms a certain angle with the first shaft.

In an embodiment, the end tool may be formed to be yaw-rotatable around the first shaft and simultaneously pitch-rotatable around the second shaft.

In an embodiment, the end tool may further include at least one pair of staple pitch main pulleys formed on one side of the one or more staple pulleys and formed to be rotatable around a second shaft that forms a certain angle with the first shaft.

In an embodiment, the end tool may further include a staple wire separation-prevention pulley that is disposed between the one or more staple pulleys and the at least one pair of staple pitch main pulleys, and formed to be rotatable around a shaft substantially the same as or parallel to the second shaft and to guide a path of the one or more staple wires.

Another embodiment of the present disclosure discloses a cartridge of a surgical instrument having an end tool rotatable in at least one direction, the cartridge including a plurality of staples disposed in an inner space of the cartridge, a reciprocating assembly formed to be movable in a first direction, which may be a longitudinal direction of the cartridge, and having a plurality of recesses formed on at least one surface thereof, and an operation member formed on one side of the reciprocating assembly, formed to be in contact with the reciprocating assembly, movable in the first direction by the reciprocating assembly, and having a retraction wire connected to one side of the operation member.

In an embodiment, the cartridge may be disposed in the accommodation part of a first jaw of the end tool.

In an embodiment, in the cartridge, the retraction wire and the operation member may be integrally formed.

In an embodiment, the cartridge may be formed to allow the retraction wire and the operation member to be attachable and detachable through fastening or unfastening.

In an embodiment, the operation member may be formed to move toward a proximal end of the end tool independently of the reciprocating assembly by pulling the retraction wire.

In an embodiment, a backward-movement prevention member may be formed on one side of the operation member to restrict a backward movement of the operation member caused by the reciprocating assembly when the reciprocating assembly moves backward in a proximal end direction.

In an embodiment, the backward-movement prevention member of the operation member may engage with an engaging part on an inner surface of a first jaw of the end tool, in which the operation member is disposed, to restrict the backward movement of the operation member.

In an embodiment, the backward-movement prevention member of the operation member may be formed to perform a rotational movement around one shaft of the operation member.

In an embodiment, the cartridge may further include a backward-movement prevention elastic member formed to restrict the rotational movement of the backward-movement prevention member.

In an embodiment, the backward-movement prevention member may be connected to a backward-movement prevention release wire.

In an embodiment, by pulling the backward-movement prevention release wire, the backward-movement prevention member may perform a rotational movement, and the restriction on the backward movement, which is caused by the reciprocating assembly, of the operation member may be released.

In an embodiment, a ratchet member of the operation member and the reciprocating assembly may be separated from each other by the retraction wire.

In an embodiment, the operation member may be formed to be movable toward a proximal end of the end tool by the retraction wire while being spaced apart from the reciprocating assembly.

In an embodiment, the cartridge may further include an operation part elastic member disposed on one side of the operation member and configured to apply an elastic force in a direction that brings a ratchet member of the operation member and the reciprocating assembly closer to each other.

In an embodiment, when the retraction wire is pulled, the ratchet member may press the operation part elastic member in a direction in which the operation part elastic member moves away from the reciprocating assembly.

In an embodiment, the reciprocating assembly may be connected to a staple drive assembly formed on the end tool, and may move in the first direction when one or more staple pulleys of the staple drive assembly rotate.

In an embodiment, when the one or more staple pulleys alternately rotate in a clockwise direction and a counterclockwise direction, the reciprocating assembly connected to the staple drive assembly may alternately move toward a distal end and a proximal end of the cartridge.

In an embodiment, the reciprocating assembly may move the operation member in contact with the reciprocating assembly in the first direction while moving toward the distal end of the cartridge.

In an embodiment, the operation member may include a body, one or more wedges formed on one side of the body and each including an inclined surface formed to have a greater height at a proximal end side of the cartridge than at a distal end side of the cartridge, a blade formed on one side of the one or more wedges and including a sharp edge, and a ratchet member formed on one side of the body and having one or more ratchets formed thereon to be in contact with the plurality of recesses of the reciprocating assembly.

In an embodiment, the cartridge may further include an operation part elastic member formed between the body or the one or more wedges and the ratchet member and configured to provide an elastic force to press the ratchet member toward the reciprocating assembly.

In an embodiment, when the retraction wire is pulled, the ratchet member may press the operation part elastic member in a direction in which the operation part elastic member moves away from the reciprocating assembly.

In an embodiment, when the reciprocating assembly moves toward a distal end of the cartridge, the reciprocating assembly may be brought into close contact with the ratchet member and may push the one or more ratchets so that the operation member moves toward the distal end of the cartridge.

In an embodiment, when the reciprocating assembly moves toward a proximal end of the cartridge, the operation member may remain stationary with respect to the at least one direction.

Another embodiment of the present disclosure discloses a method of driving a surgical instrument, the method including operations (a) in which, when a staple pulley of a staple drive assembly rotates in a first direction around a first shaft, a staple link assembly connected to the staple pulley and a reciprocating assembly of a cartridge connected to the staple link assembly move along a second shaft toward a distal end of the cartridge, (b) in which, when the reciprocating assembly moves toward the distal end of the cartridge, an operation member in contact with the reciprocating assembly may move toward the distal end of the cartridge together with the reciprocating assembly, (c) in which, as the operation member may move toward the distal end of the cartridge, the operation member ejects staples in the cartridge to the outside of the cartridge, and simultaneously, a blade of the operation member may move toward the distal end of the cartridge, (d) in which, when the staple pulley rotates in a second direction opposite to the first direction around the first shaft, the staple link assembly, which may be connected to the staple pulley, and the reciprocating assembly of the cartridge, which may be connected to the staple link assembly, move toward a proximal end of the cartridge, and (e) in which the operation member may move toward the proximal end of the cartridge independently of the reciprocating assembly by pulling a retraction wire connected to the operation member.

In an embodiment, when the staple pulley rotates in the first direction or the second direction, the reciprocating assembly may move toward the distal end of the cartridge or the proximal end of the cartridge.

In an embodiment, a bidirectional rotational movement of the staple pulley around the first shaft may be converted into a reciprocating linear movement of the reciprocating assembly, which may be connected to the staple pulley, with respect to the second shaft.

In an embodiment, the operation member may move toward the distal end of the cartridge by the reciprocating linear movement of the reciprocating assembly.

In an embodiment, a rack may be formed on one surface of the reciprocating assembly, the operation member may include a ratchet member having a ratchet formed thereon, and as the rack pushes the ratchet member while being in close contact with the ratchet member, the ratchet member may move toward the distal end of the cartridge.

In an embodiment, in operation (d), the operation member may remain stationary with respect to a direction of the second shaft.

In an embodiment, a backward-movement prevention member formed on one side of the operation member may engage with an engaging part formed on a corresponding surface to further restrict a backward movement of the operation member.

In an embodiment, when the operation member moves toward the proximal end by pulling the retraction wire, the backward-movement prevention member may rotationally move to separate from the engaging part.

In an embodiment, the method may further include an operation in which the backward-movement prevention member rotationally moves to separate from the engaging part by pulling a backward-movement prevention release wire connected to the backward-movement prevention member.

In an embodiment, the surgical instrument may further include a staple wire coupled to the staple pulley to rotate the staple pulley, and a bidirectional rotation of the staple pulley may be converted into a reciprocating linear movement of the reciprocating assembly by the staple wire.

In an embodiment, as the operation member moves toward the distal end of the cartridge, a wedge of the operation member may sequentially push and raise a plurality of staples in the cartridge to perform a stapling motion, and simultaneously the blade formed on one side of the wedge of the operation member may move toward the distal end of the cartridge to perform a cutting motion.

In an embodiment, operations (a) to (e) may be repeatedly performed.

### MODE FOR INVENTION

While the present disclosure is susceptible to various modifications and alternative forms, specific embodiments thereof are shown by way of example in the drawings and will herein be described in detail. Advantages and features of the present disclosure and methods for accomplishing the same will be more clearly understood from embodiments described below with reference to the drawings. However, the present disclosure is not limited to the embodiments disclosed below but may be implemented in various forms.

Hereinafter, the embodiments of the present disclosure will be described in detail with reference to the accompanying drawings, but when describing with reference to the drawings, equal or corresponding components will be referred to as the same reference numerals, and redundant descriptions thereof will be omitted.

In the following embodiments, the terms “first,” “second,” and the like have been used to distinguish one component from another, rather than limitative in all aspects.

In the following embodiments, singular forms are intended to include plural forms as well, unless the context clearly indicates otherwise.

In the following embodiments, terms such as “include” or “have” means that the features or components described in the specification are present, and the possibility that one or more other features or components will be added is not excluded in advance.

Sizes of components in the drawings may be exaggerated or reduced for convenience of description. For example, the size and thickness of each component shown in the drawings are arbitrarily represented for convenience of description, and thus, the present disclosure is not necessarily limited thereto.

In the following embodiments, an x-axis, a y-axis, and a z-axis are not limited to three axes of the rectangular coordinate system, and may be interpreted in a broader sense. For example, the x-axis, the y-axis, and the z-axis may be perpendicular to one another, or may represent different directions that are not perpendicular to one another.

In cases where certain embodiments may be implemented otherwise, a specific process sequence may be performed differently from the described sequence. For example, two consecutively described processes may be performed substantially at the same time or performed in an order opposite to the described order.

In a surgical instrument according to the present disclosure, when a manipulation part is rotated in one direction for at least any one of pitch, yaw, and actuation motions, an end tool is rotated in intuitively the same direction as a direction in which the manipulation part is manipulated.

FIG. 1A is a conceptual diagram of a pitch motion of a conventional surgical instrument, and FIG. 1B is a conceptual diagram of a yaw motion.

Referring to FIG. 1A, in performing a pitch motion of a conventional surgical instrument, in a state in which an end tool 120a is formed in front of a rotation center 121a of the end tool, and a manipulation part 110a is formed at the rear of a rotation center 111a of the manipulation part, when the manipulation part 110a is rotated in a clockwise direction, the end tool 120a is also rotated in the clockwise direction, and when the manipulation part 110a is rotated in a counterclockwise direction, the end tool 120a is also rotated in the counterclockwise direction. Meanwhile, referring to FIG. 1B, in performing a yaw motion of the conventional surgical instrument, in a state in which the end tool 120a is formed in front of the rotation center 121a of the end tool, and the manipulation part 110a is formed at the rear of the rotation center 111a of the manipulation part, when the manipulation part 110a is rotated in the clockwise direction, the end tool 120a is also rotated in the clockwise direction, and when the manipulation part 110a is rotated in the counterclockwise direction, the end tool 120a is also rotated in the counterclockwise direction. In this case, in view of left and right directions of a user, when the user moves the manipulation part 110a to the left, the end tool 120a is moved to the right, and when the user moves the manipulation part 110a to the right, the end tool 120a is moved to the left. As a result, a manipulation direction of the user and an operation direction of the end tool are opposite to each other, which may cause the user to make a mistake, and user's manipulation may not be easy.

FIG. 1C is a conceptual diagram of a pitch motion of another conventional surgical instrument, and FIG. 1D is a conceptual diagram of a yaw motion.

Referring to FIG. 1C, in the conventional surgical instrument, which is partially formed in a mirror symmetrical shape, in performing a pitch motion, in a state in which an end tool 120b is formed in front of a rotation center 121b of the end tool, and a manipulation part 110b is formed at the rear of a rotation center 111b of the manipulation part, when the manipulation part 110b is rotated in the clockwise direction, the end tool 120b is rotated in the counterclockwise direction, and when the manipulation part 110b is rotated in the counterclockwise direction, the end tool 120b is rotated in the clockwise direction. In this case, in view of rotation directions of the manipulation part and the end tool, a rotation direction in which the user rotates the manipulation part 110b and a rotation direction of the end tool 120b according thereto are opposite to each other. As a result, the user may be confused with the manipulation direction, and as the operation of the joint is not intuitive, the user may make an error. Further, referring to FIG. 1D, in performing a yaw motion, in a state in which the end tool 120b is formed in front of the rotation center 121b of the end tool, and the manipulation part 110b is formed at the rear of the rotation center 111b of the manipulation part, when the manipulation part 110b is rotated in the clockwise direction, the end tool 120b is rotated in the counterclockwise direction, and when the manipulation part 110b is rotated in the counterclockwise direction, the end tool 120b is rotated in the clockwise direction. In this case, in view of rotation directions of the manipulation part and the end tool, a rotation direction in which the user rotates the manipulation part 110b and a rotation direction of the end tool 120b according thereto are opposite to each other. As a result, the user may be confused with the manipulation direction, and as the operation of the joint is not intuitive, the user may make an error. In the user's pitch or yaw manipulation of the conventional surgical instrument, the user's manipulation direction and the end tool's operation direction do not match each other in view of one of the rotation direction and the left and right directions. This is because the configurations of the end tool and the manipulation part are different from each other in the joint configuration of the conventional surgical instrument. That is, this is because the manipulation part is formed at the rear of the rotation center of the manipulation part, while the end tool is formed in front of the rotation center of the end tool. In order to address the above problems, in a surgical instrument according to an embodiment of the present disclosure, which is illustrated of FIGS. 1E and 1F, an end tool 120c is formed in front of a rotation center 121c of the end tool and a manipulation part 110c is also formed in front of a rotation center 111c of the manipulation part, so that the operations of the manipulation part 110c and the end tool 120c are intuitively matched with each other. In other words, unlike existing examples such as those shown in FIGS. 1A, 1B, 1C, and ID, in which the manipulation part is close to a user with respect to the joint thereof (that is, away from the end tool), the surgical instrument according to an embodiment of the present disclosure, which is illustrated of FIGS. 1E and 1F, is formed such that at least a portion of the manipulation part is closer (than a joint thereof) to the end tool with respect to the joint thereof at any one moment or more in a manipulation process.

In other words, in the conventional surgical instrument as illustrated of FIGS. 1A, 1B, 1C, and ID, the manipulation part is formed at the rear of the rotation center thereof, while the end tool is located in front of the rotation center thereof, and thus the end tool is moved at a front side thereof with a rear side fixed through a motion of the manipulation part that is moved at a rear side thereof with a front side thereof fixed, which is not an intuitively matching structure. Accordingly, a mismatch may occur between the manipulation of the manipulation part and the motion of the end tool in view of the left and right directions or in view of the rotation direction, which may cause confusion to the user, and the manipulation of the manipulation part may be difficult to perform intuitively and quickly and may cause mistakes. In contrast, in the surgical instrument according to an embodiment of the present disclosure, since both the end tool and the manipulation part are moved with respect to the rotation center formed at the rear side thereof, it may be said that the motions are intuitively matched with each other in terms of structure. In other words, moving portions of the manipulation part are moved with respect to the rotation center formed at the rear side thereof just as moving portions of the end tool are moved with respect to the rotation center formed at the rear side thereof, and thus it may be said that the motions are intuitively matched with each other in terms of structure. This allows the user to intuitively and quickly perform a control in a direction toward the end tool, and a possibility of making a mistake may be significantly reduced. Hereinafter, a detailed mechanism enabling the above-described function will be described below.

### First Embodiment of Surgical Instrument

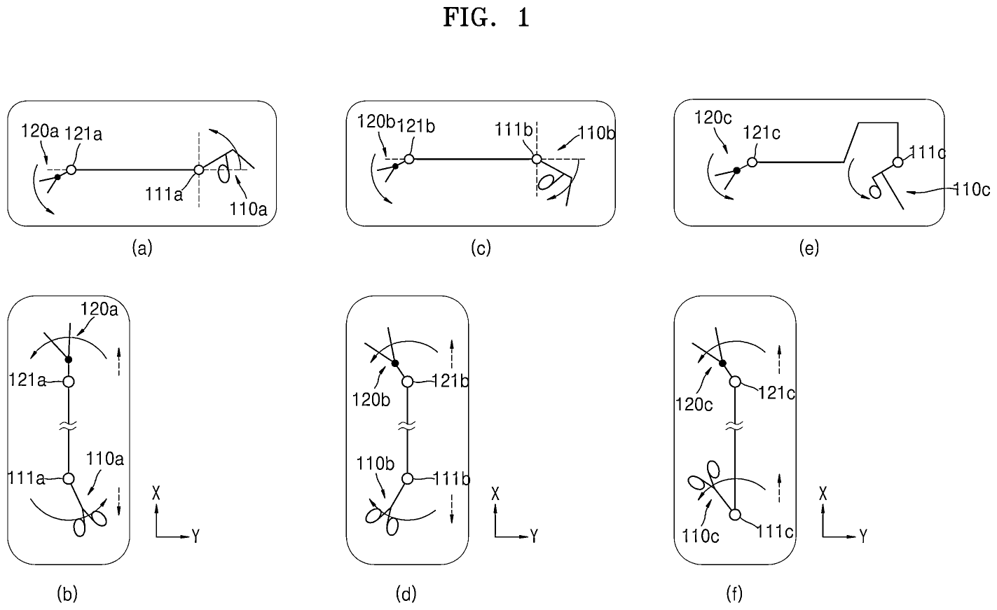

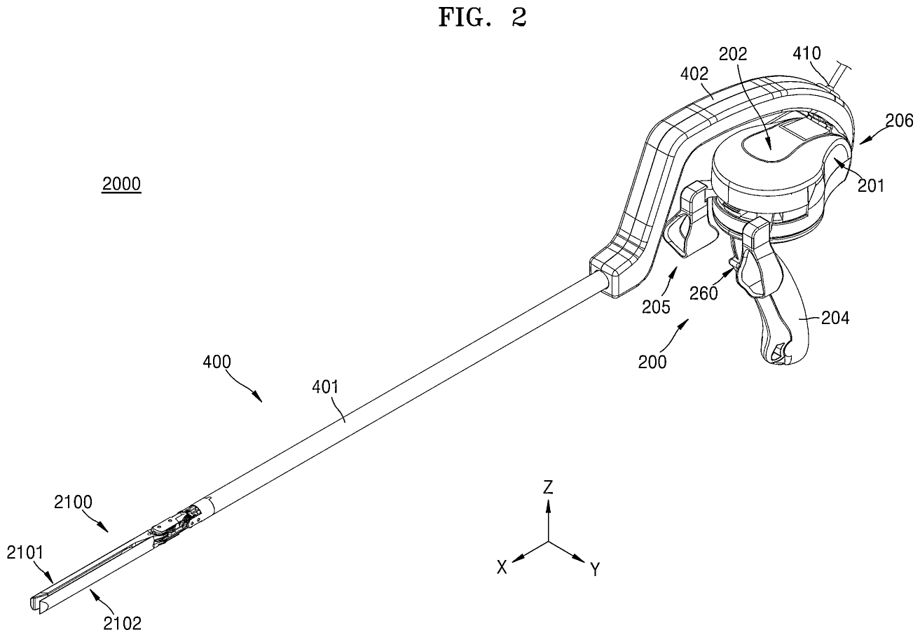

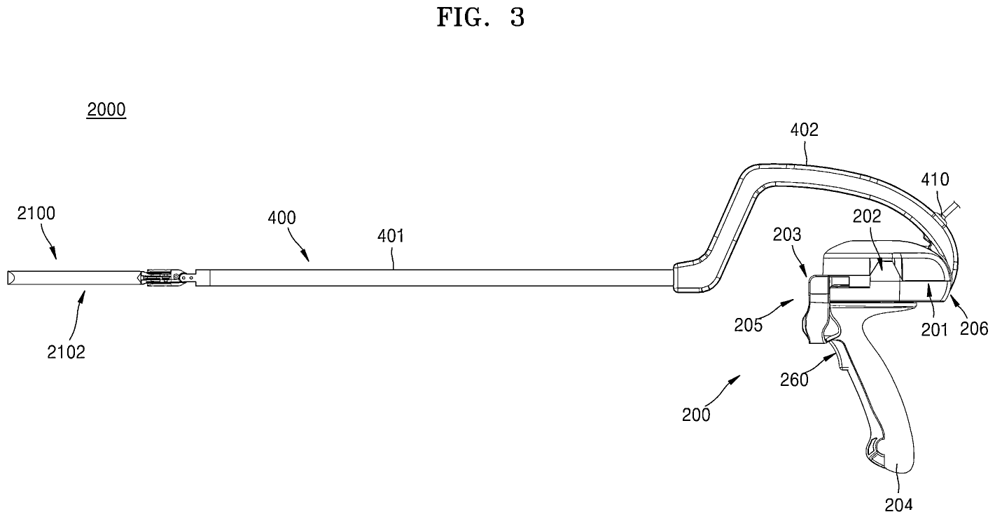

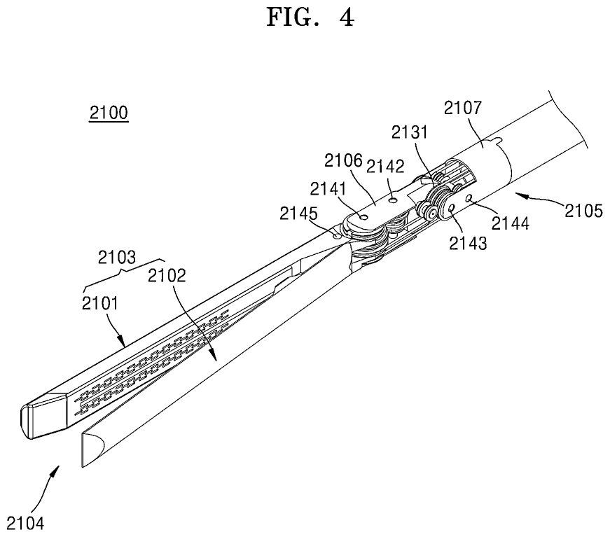

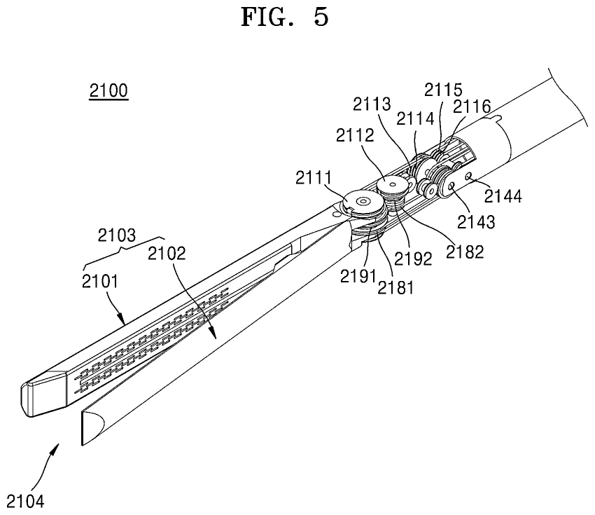

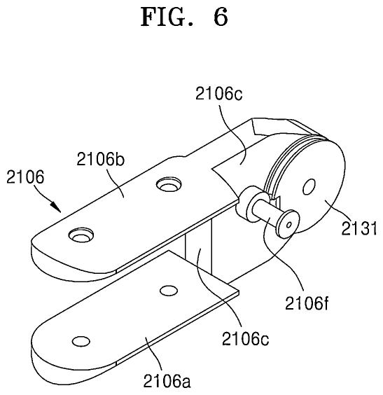

FIG. 2 is a perspective view illustrating a surgical instrument according to a first embodiment of the present disclosure, and FIG. 3 is a side view of the surgical instrument of FIG. 2. FIGS. 4 and 5 are perspective views illustrating an end tool of the surgical instrument of FIG. 2. FIG. 6 is a perspective view illustrating an end tool hub of the end tool of the surgical instrument of FIG. 2.

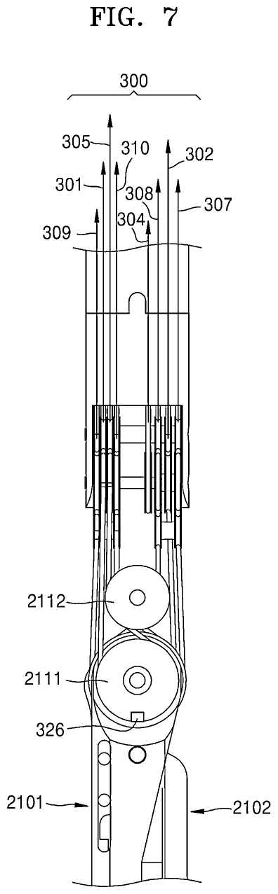

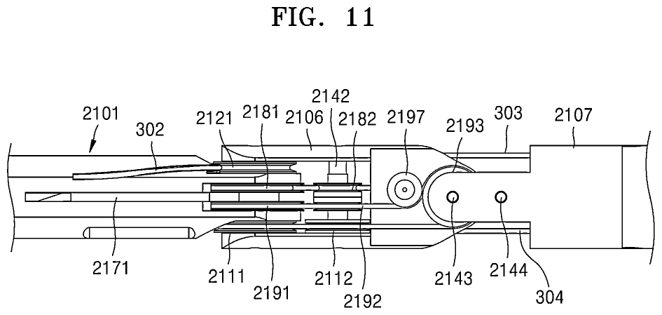

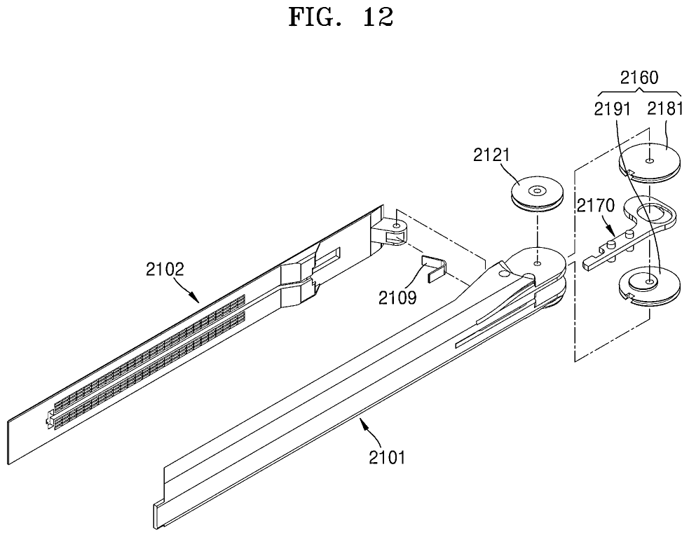

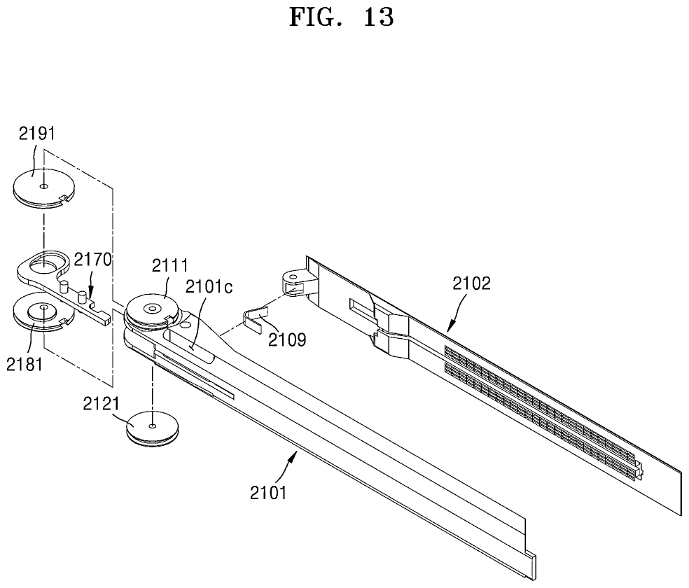

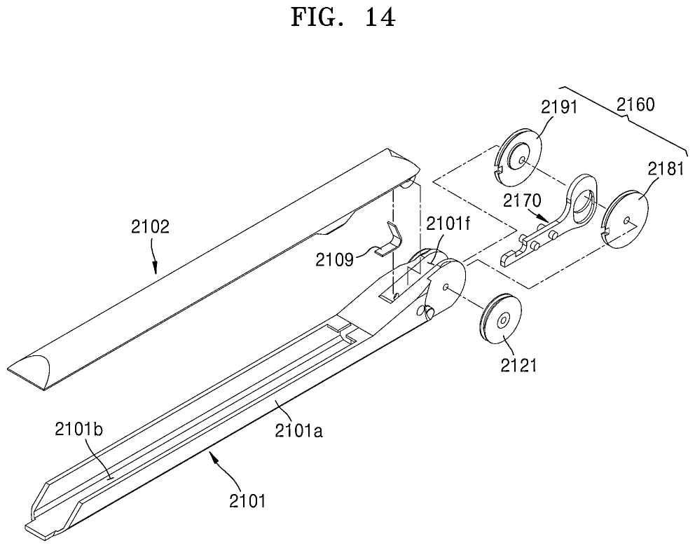

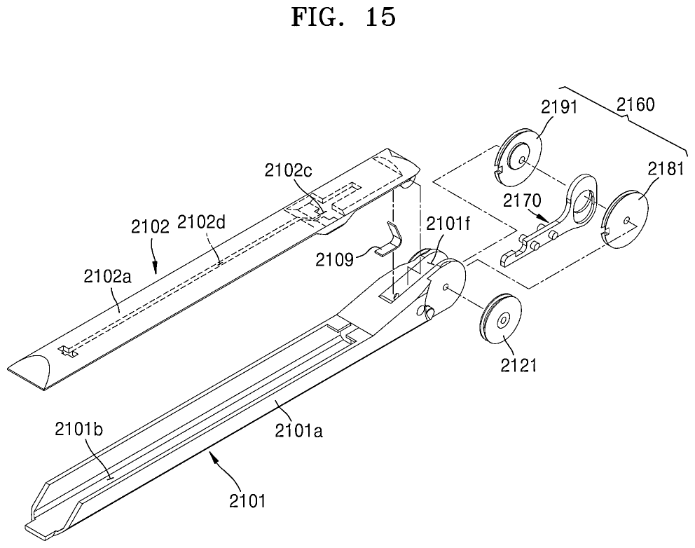

FIGS. 7 to 10 are plan views illustrating the end tool of the surgical instrument of FIG. 4. FIG. 11 is a side view illustrating the end tool of the surgical instrument of FIG. 4. FIGS. 12 to 15 are exploded perspective views of the end tool of the surgical instrument of FIG. 4.

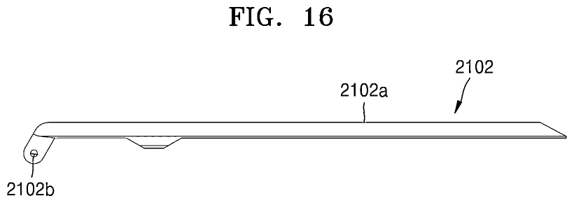

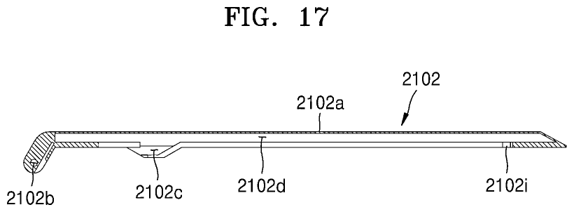

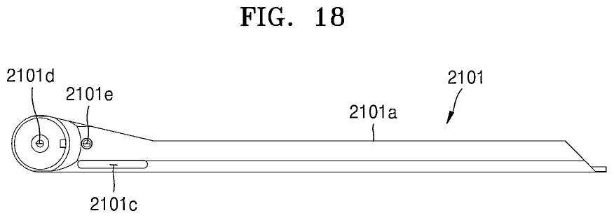

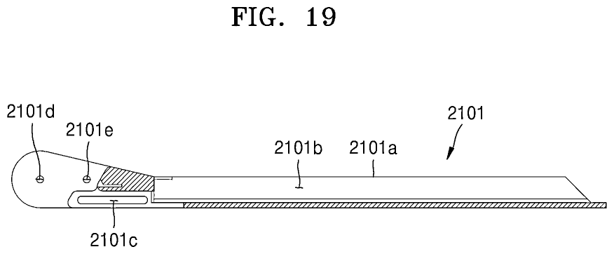

FIG. 16 is a side view illustrating a second jaw of the surgical instrument of FIG. 4, and FIG. 17 is a cross-sectional view illustrating the second jaw of the surgical instrument of FIG. 4. FIG. 18 is a side view illustrating a first jaw of the surgical instrument of FIG. 4, and FIG. 19 is a cross-sectional view illustrating the first jaw of the surgical instrument of FIG. 4.

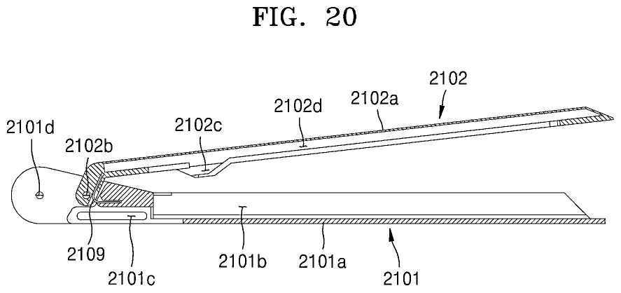

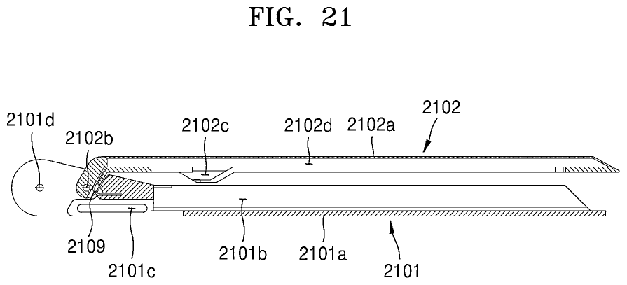

FIG. 20 is a cross-sectional view illustrating an open state of the first and second jaws of the surgical instrument of FIG. 4, and FIG. 21 is a cross-sectional view illustrating a closed state of the first and second jaws of the surgical instrument of FIG. 4.

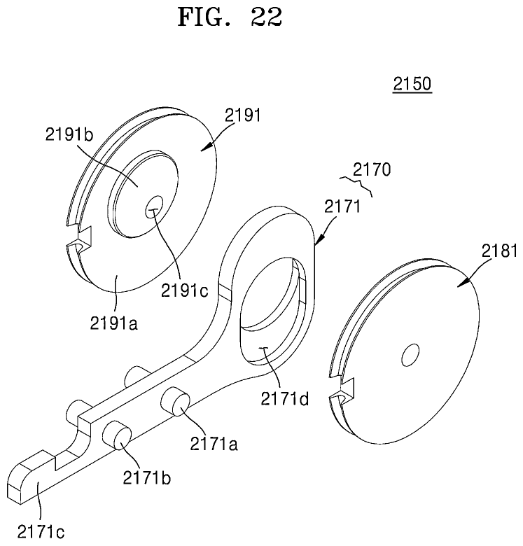

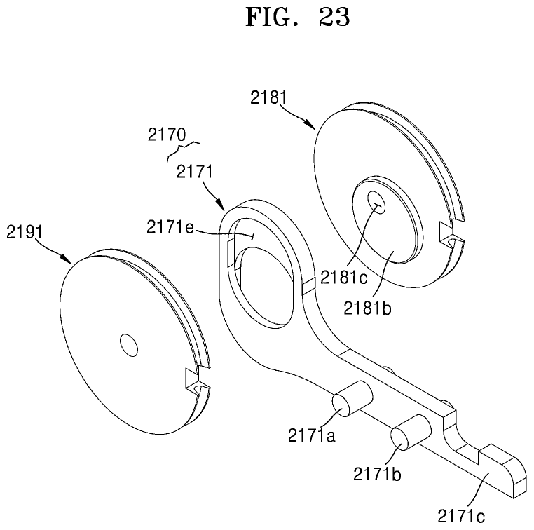

FIGS. 22 and 23 are exploded perspective views illustrating a staple drive assembly of the surgical instrument of FIG. 4.

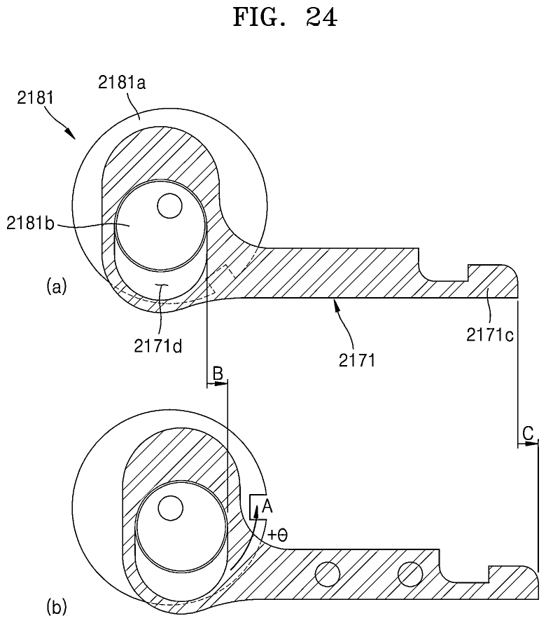

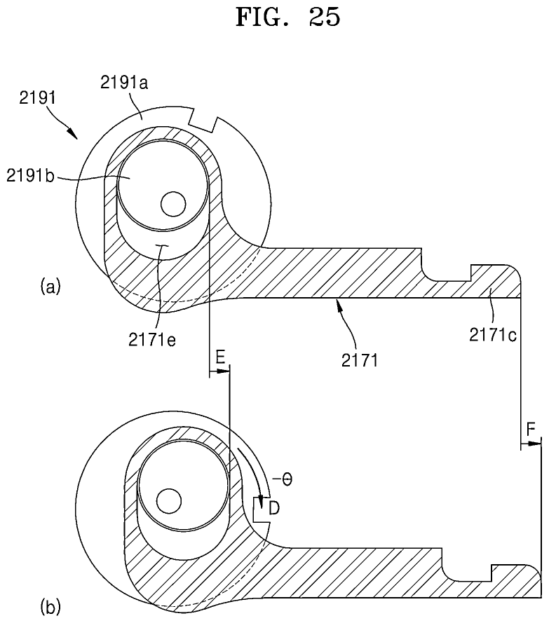

FIGS. 24 and 25 are side views illustrating an operation of the staple drive assembly of the surgical instrument of FIG. 4.

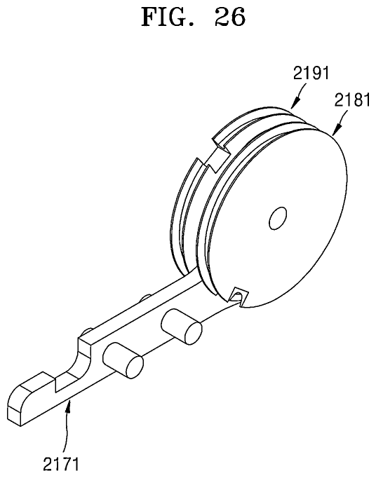

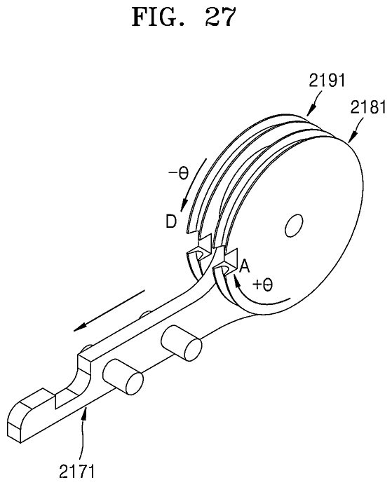

FIGS. 26 and 27 are perspective views illustrating operations of the staple drive assembly of the surgical instrument of FIG. 4.

A surgical instrument 2000 according to the first embodiment of the present disclosure includes an end tool 2100, a manipulation part 200, a power transmission part 300, and a connection part 400.

Here, the connection part 400 is formed in the shape of a hollow shaft, and one or more wires and electric wires may be accommodated therein. The manipulation part 200 is coupled to one end portion of the connection part 400, while the end tool 2100 is coupled to another end portion, and the connection part 400 may serve to connect the manipulation part 200 to the end tool 2100. Here, the connection part 400 of the surgical instrument 2000 according to the first embodiment of the present disclosure includes a straight part 401 and a bent part 402, wherein the straight part 401 is formed at a side of the connection part 400 coupled to the end tool 2100, and the bent part 402 is formed at a side pf the connection part 400 to which the manipulation part 200 is coupled. As such, since the end portion of the connection part 400 at the side of the manipulation part 200 is formed to be bent, a pitch manipulation part 201, a yaw manipulation part 202, and an actuation manipulation part 203 may be formed along an extension line of the end tool 2100 or adjacent to the extension line. In other words, it may be said that the pitch manipulation part 201 and the yaw manipulation part 202 are at least partially accommodated in a concave portion formed by the bent part 402. Due to the above-described shape of the bent part 402, the shapes and motions of the manipulation part 200 and the end tool 2100 may be further intuitively matched with each other.

Meanwhile, a plane on which the bent part 402 is formed may be substantially the same as a pitch plane, that is, an XZ plane of FIG. 2. As such, as the bent part 402 is formed on the plane substantially the same as the XZ plane, interference with the manipulation part may be reduced. Of course, for intuitive motions of the end tool and the operation part, configurations related to, but not limited to, the XZ plane are also possible.

Meanwhile, a connector 410 may be formed on the bent part 402. The connector 410 may be connected to an external power source (not shown), and the connector 410 may also be connected to the end tool 2100 via an electric wire, and may transmit, to the end tool 2100, electric energy supplied from the external power source (not shown). In addition, the electric energy transmitted to the end tool 2100 as described above may produce a driving force for rotating staple pulleys (see 2181 and 2191 of FIG. 5) to be described below in the clockwise or counterclockwise direction.

The manipulation part 200 is formed at the one end portion of the connection part 400 and provided as an interface to be directly controlled by a medical doctor, for example, a tongs shape, a stick shape, a lever shape, or the like, and when the medical doctor controls the manipulation part 200, the end tool 2100, which is connected to the interface and inserted into the body of a surgical patient, performs a certain motion, thereby performing surgery. Here, the manipulation part 200 is illustrated of FIG. 2 as being formed in a handle shape that is rotatable while the finger is inserted therein, the concept of the present disclosure is not limited thereto, and various types of manipulation parts that are connected to the end tool 2100 and manipulate the end tool 2100 may be possible.

The end tool 2100 is formed on another end portion of the connection part 400, and performs necessary motions for surgery by being inserted into a surgical site. As an example of the end tool 2100, a pair of jaws 2103 for performing a grip motion may be used. However, the concept of the present disclosure is not limited thereto, and various devices for performing surgery may be used as the end tool 2100. For example, a configuration such as a cantilever cautery may also be used as the end tool. The end tool 2100 is connected to the manipulation part 200 by the power transmission part 300, and receives a driving force of the manipulation part 200 through the power transmission part 300 to perform a motion necessary for surgery, such as gripping, cutting, suturing, or the like.

Here, the end tool 2100 of the surgical instrument 2000 according to the first embodiment of the present disclosure is formed to be rotatable in at least one direction, for example, the end tool 2100 may perform a pitch motion around a Y-axis of FIG. 2 and simultaneously perform a yaw motion and an actuation motion around a Z-axis of FIG. 2.

Here, each of the pitch, yaw, and actuation motions used in the present disclosure are defined as follows.

First, the pitch motion means a motion of the end tool 2100 rotating in a vertical direction with respect to an extension direction of the connection part 400 (an X-axis direction of FIG. 2), that is, a motion rotating around the Y-axis of FIG. 2. In other words, the pitch motion means a motion of the end tool 2100, which is formed to extend from the connection part 400 in the extension direction of the connection part 400 (the X-axis direction of FIG. 2), rotating vertically around the Y-axis with respect to the connection part 400.

Next, the yaw motion means a motion of the end tool 2100 rotating in the left and right directions, that is, a motion rotating around the Z-axis of FIG. 2, with respect to the extension direction of the connection part 400 (the X-axis direction of FIG. 2). In other words, the yaw motion means a motion of the end tool 2100, which extends from the connection part 400 in the extension direction of the connection part 400 (the X-axis direction of FIG. 2), rotating horizontally around the Z-axis with respect to the connection part 400. That is, the yaw motion means a motion of the two jaws 2103, which are formed on the end tool 2100, rotating around the Z-axis in the same direction.

Meanwhile, the actuation motion means a motion of the end tool 2100 rotating around the same shaft of rotation as that of the yaw motion, while the two jaws 2103 rotate in opposite directions so as to be closed or opened. That is, the actuation motion means a motion of the two jaws 2103, which are formed on the end tool 2100, rotating in the opposite directions around the Z-axis.

The power transmission part 300 may serve to connect the manipulation part 200 to the end tool 2100 to transmit the driving force of the manipulation part 200 to the end tool 2100, and may include a plurality of wires, pulleys, links, sections, gears, and the like.

The end tool 2100, the manipulation part 200, the power transmission part 300, and the like of the surgical instrument 2000 of FIG. 2 will be described in detail later.

### (Intuitive Driving)

Hereinafter, intuitive driving of the surgical instrument 2000 of the present disclosure will be described.

First, while holding a first handle 204 with the palm of the hand, the user may rotate the first handle 204 around the Y-axis to perform a pitch motion, and rotate the first handle 204 around the Z-axis to perform a yaw motion. In addition, the user may perform an actuation motion by manipulating the actuation manipulation part 203 in a state in which the thumb and the index finger are inserted into a hand ring-shaped actuation extension part formed at one end portion of the actuation manipulation part 203.

Here, in the surgical instrument 2000 according to the first embodiment of the present disclosure, when the manipulation part 200 is rotated in one direction with respect to the connection part 400, the end tool 2100 is rotated in a direction that is intuitively the same as a manipulation direction of the manipulation part 200. In other words, when the first handle 204 of the manipulation part 200 is rotated in one direction, the end tool 2100 is also rotated in a direction intuitively the same as the one direction, so that the pitch motion or the yaw motion is performed. Here, the phrase “intuitively the same direction” may be further explained as meaning that a direction of movement of the user's finger gripping the manipulation part 200 and a direction of movement of a distal end of the end tool 2100 form substantially the same direction. Of course, “the same direction” as used herein may not be a perfectly matching direction on a three-dimensional coordinate, and may be understood to be equivalent to the extent that, for example, when the user's finger moves to the left, the distal end of the end tool 2100 is moved to the left, and when the user's finger moves down, the distal end of the end tool 2100 is moved down.

In addition, to this end, in the surgical instrument 2000 according to the first embodiment of the present disclosure, the manipulation part 200 and the end tool 2100 are formed in the same direction with respect to a plane perpendicular to the extension axis (X-axis) of the connection part 400. That is, when viewed based on a YZ plane of FIG. 2, the manipulation part 200 is formed to extend in a positive (+) X-axis direction, and the end tool 2100 is also formed to extend in the positive (+) X-axis direction. In other words, it may be said that a formation direction of the end tool 2100 on one end portion of the connection part 400 is the same as a formation direction of the manipulation part 200 on another end portion of the connection part 400 on the basis of the YZ plane. Alternatively, in other words, it may be said that the manipulation part 200 may be formed in a direction away from the body of a user gripping the manipulation part 200, that is, in a direction in which the end tool 2100 is formed. That is, the first handle 204, the actuation manipulation part, or the like, which the user grips and moves to perform the actuation, yaw, and pitch motions, is formed such that the portion that moves to perform each motion extends in the positive (+) X-axis direction beyond the center of rotation of each joint for that motion. In this manner, the manipulation part 200 may be configured in the same manner as the end tool 2100 in which each moving portion is formed to extend in the positive (+) X-axis direction from the rotation center of the corresponding joint for the motion, and as described with reference to FIG. 1, the manipulation direction of the user may be identical to the operation direction of the end tool from the viewpoint of the rotation directions and the left and right directions. As a result, intuitively the same manipulation may be achieved.

In detail, in the case of the conventional surgical instrument, a direction in which a user manipulates the manipulation part is different from a direction in which the end tool is actually operated, that is, intuitively different from the direction in which the end tool is actually operated, and thus, a surgical operator may not easily intuitively manipulate the surgical instrument and may spend a long time to learn a skill of operating the end tool in desired directions, and in some cases, malfunctions may occur, which may cause damage to patients.

In order to address such problems, the surgical instrument 2000 according to the first embodiment of the present disclosure is configured such that the manipulation direction of the manipulation part 200 and the operation direction of the end tool 2100 are intuitively identical to each other. To this end, the manipulation part 200 is configured similar to the end tool 2100, that is, in the manipulation part 200, portions that are actually moved for actuation, yaw, and pitch motions are formed to extend respectively from rotation centers of the corresponding joints in the positive (+) X-axis direction.

Hereinafter, the end tool 2100, the manipulation part 200, the power transmission part 300, and the like of the surgical instrument 2000 of FIG. 2 will be described in more detail.

### (Power Transmission Part)

Hereinafter, the power transmission part 300 of the surgical instrument 2000 of FIG. 2 will be described in more detail.

Referring to FIGS. 2 to 11 and the like, the power transmission part 300 of the surgical instrument 2000 according to an embodiment of the present disclosure may include a wire 301, a wire 302, a wire 303, a wire 304, a wire 305, a wire 307, a wire 308, a wire 309, and a wire 310.

Here, the wire 301 and the wire 305 may be paired to serve as first jaw wires. In addition, in the present embodiment, the second jaw wire may not be provided, and thus,

the wire 301 and the wire 305, which are first jaw wires, may be referred to as jaw wires. The wire 303 and the wire 304 may be paired to serve as pitch wires.

In addition, the wire 307 and the wire 308 may be paired to serve as first staple wires. The wire 309 and the wire 310 may be paired to serve as second staple wires. Here, a component encompassing the wires 307 and 308, which are first staple wires, and the wires 309 and 310, which are second staple wires, may be referred to as a staple wire.

In addition, the power transmission part 300 of the surgical instrument 2000 according to an embodiment of the present disclosure may include one or more coupling members (e.g., 326) coupled to an end portion of each of the wires in order to couple the wires to the pulleys. Here, each of the coupling members may have various shapes as necessary, such as a ball shape, a tube shape, and the like.

An example of the coupling relationship between the wires, the coupling members, and the respectively pulleys will be described as follows.

First, the wires 301 and 305, which are first jaw wires, may be a single wire. A first jaw wire-end tool coupling member is inserted at an intermediate point of the first jaw wire, which is a single wire, and both strands of the first jaw wire centered on the coupling member may be referred to as the wire 301 and the wire 305, respectively.

Alternatively, the wires 301 and 305, which are first jaw wires, may also be formed as separate wires and connected to each other by a coupling member. In addition, by coupling the coupling member to the pulley 2111, the wires 301 and 305 may be fixedly coupled to the pulley, thereby allowing the pulley 2111 to rotate as the wires 301 and 305 are pulled and released.

In addition, by coupling the first jaw wire-manipulation part coupling member to the pulley as described above, the wires 301 and 305 may be fixedly coupled to the pulley. As a result, when the pulley is rotated by a motor or human force, the wires 301 and 305 are pulled and released, thereby allowing the pulley 2111 of the end tool 2100 to rotate.

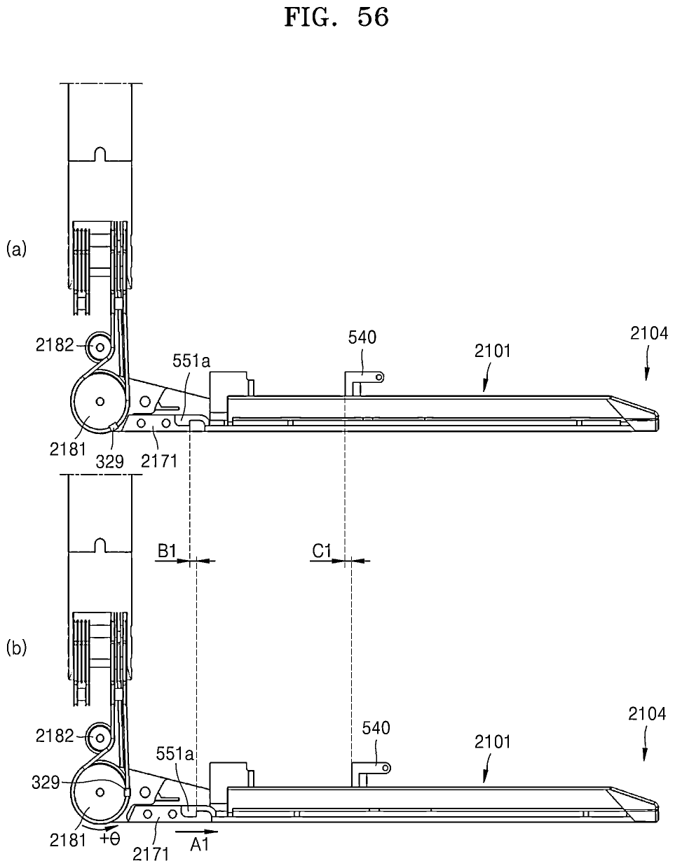

In the same manner, the wires 307 and 308, which are first staple wires, are respectively coupled to a coupling member (see 329 of FIG. 56), which is a staple wire-end tool coupling member, and a staple wire-manipulation part coupling member (not shown). In addition, the coupling member (see 329 of FIG. 56) is coupled to a first staple pulley 2181, and the staple wire-manipulation part coupling member (not shown) is coupled to a pulley (not shown) on a side of the manipulation part. As a result, when the pulley on the side of the manipulation part is rotated by a motor or a human force, the first staple pulley 2181 of the end tool 2100 may be rotated as the wires 307 and 308 are pulled and released.

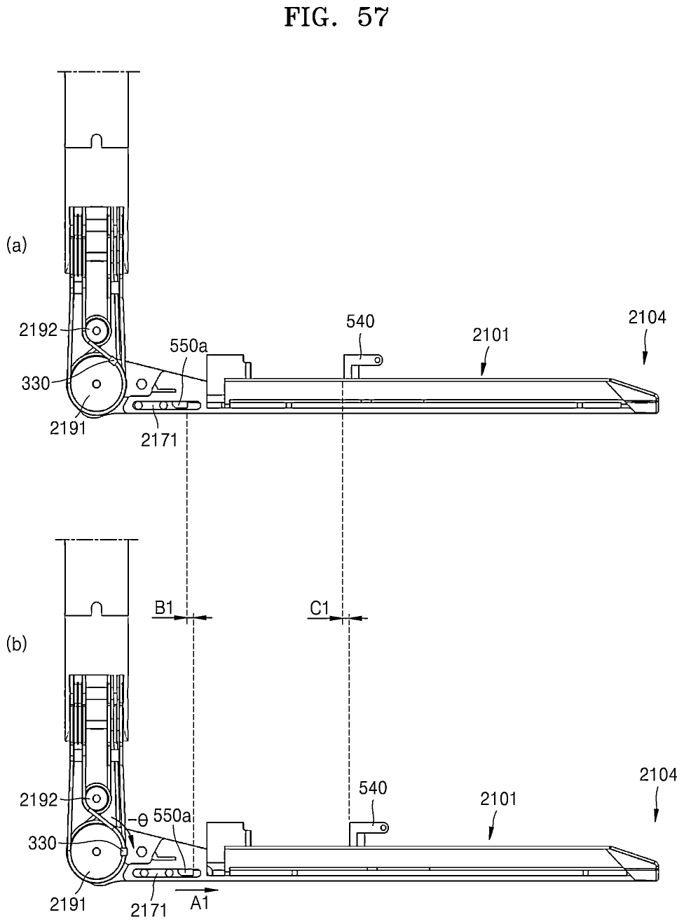

In the same manner, the wire 309 and the wire 310, which are second staple wires, are respectively coupled to a coupling member (see 330 of FIG. 57), which is a staple wire-end tool coupling member, and the staple wire-manipulation part coupling member (not shown). In addition, the coupling member (see 330 of FIG. 57) is coupled to a second staple pulley 2191, and the staple wire-manipulation part coupling member (not shown) is coupled to the pulley (not shown) on the side of the manipulation part. As a result, when the pulley on the side of the manipulation part is rotated by a motor or a human force, the second staple pulley 2191 of the end tool 2100 may be rotated as the wires 309 and 310 are pulled and released.

### (End Tool)

Hereinafter, the end tool 2100 of the surgical instrument 2000 of FIG. 2 will be described in more detail.

FIGS. 4 and 5 are perspective views illustrating the end tool of the surgical instrument of FIG. 2, FIG. 6 is a perspective view illustrating the end tool hub of the end tool of the surgical instrument of FIG. 2, and FIGS. 7 to 10 are plan views illustrating the end tool of the surgical instrument of FIG. 4.

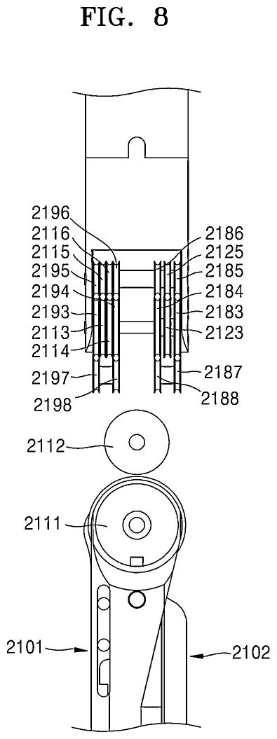

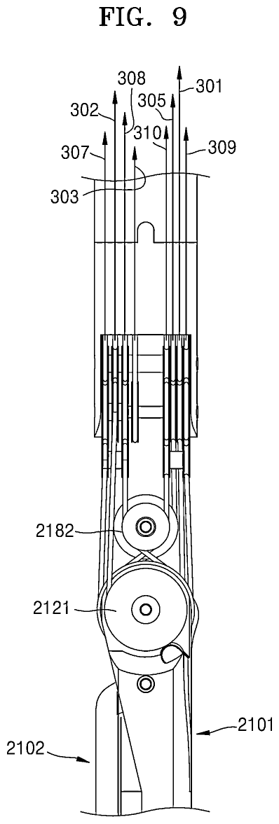

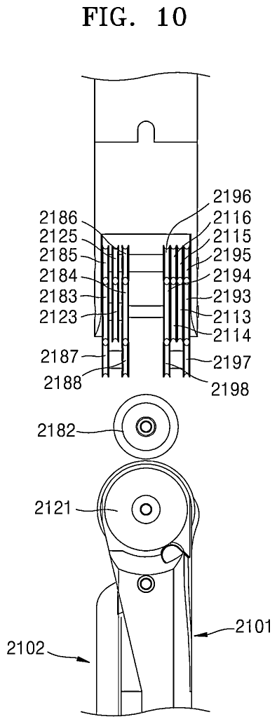

Here, FIG. 4 illustrates a state in which an end tool hub 2106 and a pitch hub 2107 are coupled to the end tool, and FIG. 5 illustrates a state in which the end tool hub 2106 is removed from the end tool. Meanwhile, FIG. 7 is a view mainly illustrating the wires, and FIG. 8 is a view mainly illustrating the pulleys. Meanwhile, FIGS. 9 and 10 are plan views viewed from a different direction than that of FIG. 7. FIG. 9 is a view mainly illustrating the wires, and FIG. 10 is a view mainly illustrating the pulleys.

The end tool 2100 of the first embodiment of the present disclosure includes a pair of jaws for performing a grip motion, that is, a first jaw 2101 and a second jaw 2102. Here, each of the first jaw 2101 and the second jaw 2102, or a component encompassing the first jaw 2101 and the second jaw 2102 may be referred to as the jaw 2103.

Further, the end tool 2100 may include the pulley 2111, a pulley 2112, a pulley 2113, a pulley 2114, a pulley 2115, and a pulley 2116 that are related to a rotational motion of the first jaw 2101.

In addition, the end tool 2100 may include a pulley 2121, a pulley 2122, a pulley 2123, and a pulley 2125 that are related to a rotational movement of the retraction wire 302.

Here, the pulleys facing each other are illustrated in the drawings as being formed parallel to each other, but the concept of the present disclosure is not limited thereto, and each of the pulleys may be variously formed with a position and a size suitable for the configuration of the end tool.

Further, the end tool 2100 of the first embodiment of the present disclosure may include the end tool hub 2106 and the pitch hub 2107.

A rotation shaft 2141 and a rotation shaft 2142 may be inserted through the end tool hub 2106, and the end tool hub 2106 may internally accommodate at least some of the pulley 2111 and the pulley 2121, which are axially coupled to the rotation shaft 2141. In addition, the end tool hub 2106 may internally accommodate at least some of the pulley 2112 and the pulley 2122 that are axially coupled to the rotation shaft 2142.

In detail, referring to FIG. 6, the end tool hub 2106 includes a first jaw pulley coupling part 2106a, a second pulley coupling part 2106b, a guide part 2106c, a pitch pulley coupling part 2106e, and a separation-prevention-pulley coupling part 2106f.

In detail, the first jaw pulley coupling part 2106a and the second pulley coupling part 2106b are formed to face each other, and the pulley 2111, the pulley 2121, and the staple pulleys 2181 and 2191 are accommodated inside the first jaw pulley coupling part 2106a and the second pulley coupling part 2106b. In addition, a through hole is formed in each of the first jaw pulley coupling part 2106a and the second pulley coupling part 2106b such that the rotation shaft 2141 passes through and axially couples the first jaw pulley coupling part 2106a, the pulley 2111, the staple pulleys 2181 and 2191, the pulley 2121, and the second pulley coupling part 2106b.

The first jaw pulley coupling part 2106a and the second pulley coupling part 2106b are connected to each other by the guide part 2106c. For example, the first jaw pulley coupling part 2106a and the second pulley coupling part 2106b parallel to each other are coupled to each other by the guide part 2106c formed in a direction substantially perpendicular thereto, so that the first jaw pulley coupling part 2106a, the second pulley coupling part 2106b, and the guide part 2106c form a substantially “U-shape,” in which the pulley 2111, the pulley 2121, and the staple pulleys 2181 and 2191 are accommodated.

Here, the pulley 2111, which is a first jaw pulley, is disposed adjacent to the first jaw pulley coupling part 2106a of the end tool hub 2106, and the pulley 2121, which is a pulley for the retraction wire 302, is disposed adjacent to the second pulley coupling part 2106b of the end tool hub 2106, so that the staple pulleys 2181 and 2191 and at least a portion of a staple link assembly 2170 may be disposed between the first jaw pulley coupling part 2106a and the second pulley coupling part 2106b.

As described above, in the present disclosure, a stapling and cutting motion can be performed using the staple pulleys 2181 and 2191 and the staple link assembly 2170, along with the pitch and yaw motions of the end tool 2100, by arranging the staple pulleys 2181 and 2191 and at least a portion of the staple link assembly 2170 between the pulley 2111, which is a first jaw pulley, and the pulley 2121, which is a pulley for the retraction wire 302.

In an optional embodiment, the pulley 2121 may be defined simply as a pulley that is opposite to the pulley 2111, which is a first jaw pulley, and functions as something other than the pulley for the retraction wire. For example, the pulley 2121, which is an opposing pulley, may also function as a second jaw pulley. That is, in the end tool 2100 of the present embodiment, when the second jaw 2102 is coupled to the first jaw 2101, and the second jaw 2102 rotates together with the first jaw 2101, an additional second jaw pulley may not be necessary. However, as another example, in an alternative configuration in which the second jaw 2102 is coupled to a separate second jaw pulley similar to the first jaw 2101 (for example, refer to an end tool 5100 in a fourth embodiment to be described later), the pulley 2121 may function as the second jaw pulley, and in this case, the staple pulleys 2181 and 2191 and at least a portion of the staple link assembly 2170 can be disposed between the first jaw pulley and the second jaw pulley.

Meanwhile, a pulley 2131 serving as an end tool pitch pulley may be formed at one end portion of the end tool hub 2106. As shown in FIG. 6, the pulley 2131 may be integrally formed with the end tool hub 2106 as one body. That is, a disk-shaped pulley is formed at one end portion of the end tool hub 2106, and a groove around which a wire may be wound may be formed on an outer circumferential surface of the pulley. Alternatively, the pulley 131 may be formed as a separate member from the end tool hub 2106 to be coupled to the end tool hub 2106.

The wires 303 and 304 are coupled to the pulley 2131 serving as an end tool pitch pulley, and a pitch motion is performed as the pulley 2131 is rotated around a rotation shaft 2143.

Meanwhile, the separation-prevention-pulley coupling part 2106f may be further formed on one side of the pulley 2131. The separation-prevention-pulley coupling part 2106f is formed parallel to the rotation shaft 2143, which is an end tool pitch rotation shaft, to allow a pulley 2187, a pulley 2188, a pulley 2197, and a pulley 2198, which will be described later, to be coupled thereto. Here, the pulley 2187 and the pulley 2188 may function as first-staple-wire-separation-prevention pulleys, and the pulley 2197 and the pulley 2198 may function as second staple wire separation-prevention pulleys. This will be described in more detail later.

The rotation shaft 2143 and a rotation shaft 2144 are inserted through the pitch hub 2107, and the pitch hub 2107 may be axially coupled to the end tool hub 2106/pulley 2131 by the rotation shaft 2143. Thus, the end tool hub 2106 and the pulley 2131 may be formed to be rotatable around the rotation shaft 2143 with respect to the pitch hub 2107.

Further, the pitch hub 2107 may internally accommodate at least some of the pulley 2113, the pulley 2114, and the pulley 2123 that are axially coupled to the rotation shaft 2143. In addition, the pitch hub 2107 may internally accommodate at least some of the pulley 2115, the pulley 2116, and the pulley 2125 that are axially coupled to the rotation shaft 2144.

As described above, the end tool 2100 of the first embodiment of the present disclosure may include the rotation shaft 2141, the rotation shaft 2142, the rotation shaft 2143, and the rotation shaft 2144, the rotation shaft 2141 and the rotation shaft 2142 may be inserted through the end tool hub 2106, and the rotation shaft 2143 and the rotation shaft 2144 may be inserted through the pitch hub 2107.

The rotation shaft 2141, the rotation shaft 2142, the rotation shaft 2143, and the rotation shaft 2144 may be arranged sequentially from a distal end 2104 toward a proximal end 2105 of the end tool 2100. Accordingly, starting from the distal end 2104, the rotation shaft 2141 may be referred to as a first pin, the rotation shaft 2142 may be referred to as a second pin, the rotation shaft 2143 may be referred to as a third pin, and the rotation shaft 2144 may be referred to as a fourth pin.

Here, the rotation shaft 2141 may function as an end tool jaw pulley rotation shaft, the rotation shaft 2142 may function as an end tool jaw auxiliary pulley rotation shaft, the rotation shaft 2143 may function as an end tool pitch rotation shaft, and the rotation shaft 2144 may function as an end tool pitch auxiliary rotation shaft of the end tool 2100.

One or more pulleys may be inserted into each of the rotation shafts 2141 2142, 2143, and 2144.

Meanwhile, a rotation shaft 2145 may be further formed on one side of the rotation shaft 2141, specifically, one side of the rotation shaft 2141 at the distal end 2104 side. The rotation shaft 2145 may be inserted through the first jaw 2101 and the second jaw 2102, so that the second jaw 2102 may function as a jaw rotation shaft that rotates with respect to the first jaw 2101.

The pulley 2111 functions as an end tool first jaw pulley, and the pulley 2121 functions as a pulley for the retraction wire 302.

The pulley 2111 may be referred to as a first jaw pulley, an end tool jaw pulley, or simply a jaw pulley.

The pulley 2111, which is an end tool jaw pulley, and the pulley 2121, which is a pulley for the retraction wire 302, are formed to face each other, and are formed to be rotatable independently of each other around the rotation shaft 2141, which is an end tool jaw pulley rotation shaft. At this time, the pulleys 2111 and 2121 are formed to be spaced apart to a certain distance, allowing an accommodation part for the staple pulleys 2181 and 2191 and the staple link assembly 2170 to be formed therebetween, so that the staple pulleys 2181 and 2191 and at least a portion of the staple link assembly 2170 may be disposed in the accommodation part.

Here, in the drawings, it is illustrated that the pulley 2111 and the pulley 2121 are formed to rotate around one rotation shaft 2141, but it is of course possible that each pulley may be formed to be rotatable around a separate shaft. Here, the first jaw 2101 is fixedly coupled to the pulley 2111 and rotates together with the pulley 2111. For example, the pulley 2111 may be formed integrally with the first jaw 2101.

As the retraction wire 302 is wound around the pulley 2121, and the retraction wire 302 wound around the pulley 2121 is pulled toward the proximal end, an operation member 540 may be moved backward. This will be described later.

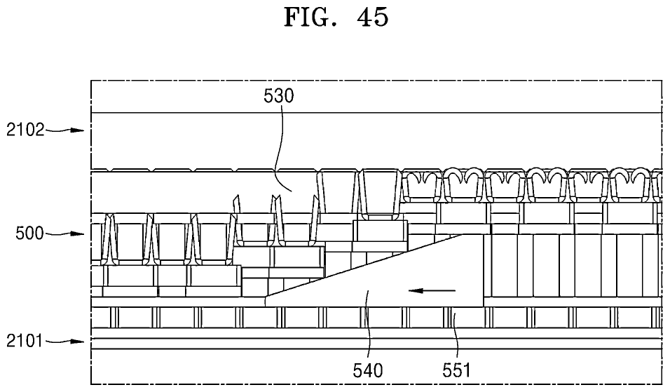

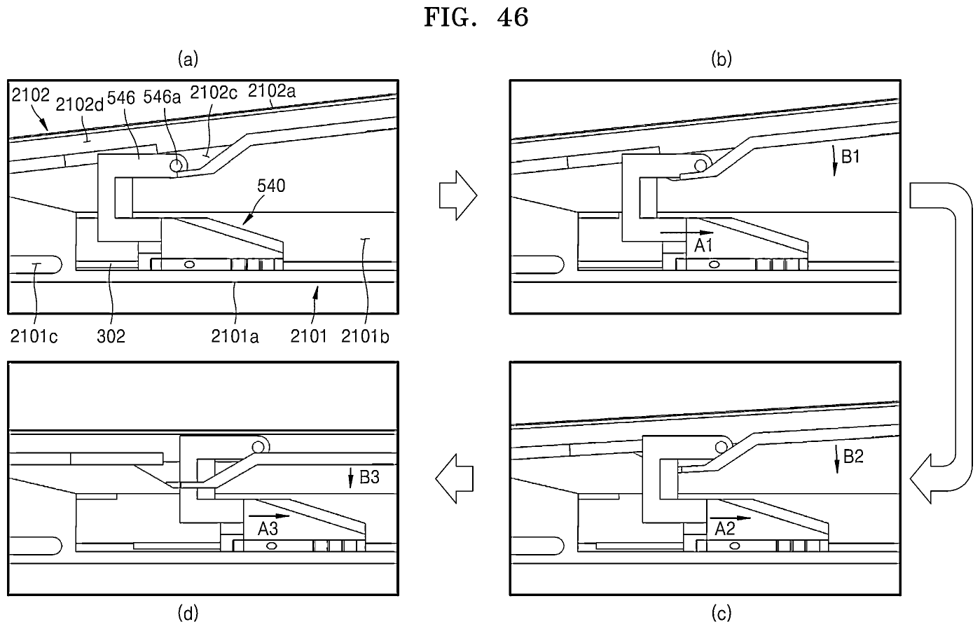

The end tool 2100 may perform a yaw motion in response to the rotation of the pulley 2111. In addition to this yaw motion, the end tool 2100 of the present embodiment may perform actuation without the rotation of the pulley. For example, due to an elastic member 2109, which will be described later, the second jaw 2102 may maintain an open state with respect to the first jaw 2101 as a default state thereof, and as the operation member 540 moves, a clamp 546 is coupled to the second jaw 2102 and, more specifically moves, into the interior of the second jaw 2102 through a guide part 2102c of the second jaw 2102, so that, as the clamp 546 moves forward (see FIG. 46 to be described later), the first jaw 2101 and the second jaw 2102 may naturally maintain a closed state.

Here, in the drawing, the first jaw 2101 and the pulley 2111 are illustrated as being integrally formed as one body. As another example, the first jaw 2101 and the pulley 2111 may be formed as separate members and then coupled together.

The pulley 2112 may function as an end tool first jaw auxiliary pulley.

In detail, the pulley 2112, which is an auxiliary pulley, may be additionally provided on one side of the pulley 2111, in other words, the pulley 2112, which is an auxiliary pulley, may be disposed between the pulley 2111 and the pulley 2113/pulley 2114. The pulley 2112 may be formed to be rotatable independently of each other around the rotation shaft 2142. The pulley 2113 and the pulley 2114 may function as end tool first jaw pitch main pulleys.

The pulley 2115 and the pulley 2116 may function as end tool first jaw pitch sub-pulleys.

Hereinafter, components related to the rotation of the pulley 2111 will be described.

The pulley 2113 and the pulley 2114 function as end tool first jaw pitch main pulleys. That is, the pulley 2113 and the pulley 2114 function as main rotation pulleys for a pitch motion of the first jaw 2101. Here, the wire 301, which is a first jaw wire, is wound around the pulley 2113, and the wire 305, which is a first jaw wire, is wound around the pulley 2114.

The pulley 2115 and the pulley 2116 function as end tool first jaw pitch sub-pulleys. That is, the pulley 2115 and the pulley 2116 function as sub-rotation pulleys for a pitch motion of the first jaw 2101. Here, the wire 301, which is a first jaw wire, is wound around the pulley 2115, and the wire 305, which is a first jaw wire, is wound around the pulley 2116.

Here, the pulley 2113 and the pulley 2114 are disposed on one side of the pulley 2111 to face each other. Here, the pulley 2113 and the pulley 2114 are formed to be rotatable independently of each other around the rotation shaft 2143 that is an end tool pitch rotation shaft. In addition, the pulley 2115 and the pulley 2116 are disposed on one side of the pulley 2113 and one side of the pulley 2114, respectively, to face each other. Here, the pulley 2115 and the pulley 2116 are formed to be rotatable independently of each other around the rotation shaft 2144 that is an end tool pitch auxiliary rotation shaft. Here, in the drawings, it is illustrated that the pulley 2113, the pulley 2115, the pulley 2114, and the pulley 2116 are all formed to be rotatable around a Y-axis direction, but the concept of the present disclosure is not limited thereto, and the rotation shafts of the respective pulleys may be formed in various directions according to configurations thereof.

The wire 301, which is a first jaw wire, is sequentially wound to make contact with at least portions of the pulley 2115, the pulley 2113, and the pulley 2111. In addition, the wire 305 connected to the wire 301 by the coupling member is sequentially wound to make contact with at least portions of the pulley 2111, the pulley 2112, the pulley 2114, and the pulley 2116.

In other words, the wires 301 and 305, which are first jaw wires, are sequentially wound to make contact with at least portions of the pulley 2115, the pulley 2113, the pulley 2111, the pulley 2112, the pulley 2114, and the pulley 2116 and are formed to move along the above pulleys while rotating the above pulleys.

Accordingly, when the wire 301 is pulled in the direction of an arrow 301 of FIG. 7, the coupling member to which the wire 301 is coupled and the pulley 2111 coupled thereto rotate in one direction, and when the wire 305 is pulled in the direction of an arrow 305 of FIG. 7, the coupling member to which the wire 305 is coupled and the pulley 2111 coupled thereto rotate in a direction opposite to the one direction.

Hereinafter, the pulley 2112 serving as an auxiliary pulley will be described in more detail.

The pulley 2112 comes into contact with the wire 305, which is a first jaw wire, and changes the arrangement path of the wire 305 to a certain extent. In this manner, the pulley 2112 serves to increase an rotation angle of each of the first jaw 2101 and the second jaw 2102.

That is, when the auxiliary pulley is not disposed, the pulley 2103 may be rotated only up to a right angle, but in an embodiment of the present disclosure, by additionally providing the pulley 2112, which is an auxiliary pulley, the maximum rotation angle may be increased. This enables the two jaws of the end tool 2100 to perform the motion of opening for an actuation motion while being yaw-rotated together by 90° in one direction. Through the pulley 2112, the range of yaw rotation that enables the actuation motion can be expanded.

This will be described below in more detail.

In the case of the surgical instrument 2000 of the present disclosure, the pulley 2112, which is an auxiliary pulley, is additionally disposed on one side of the pulley 2111. As such, by changing the arrangement path of the wire 305, which is a first jaw wire, to a certain extent through the arrangement of the pulley 2112, a tangential direction of the wire 305 is changed, allowing the coupling member 323, which couples the wire 301 to the pulley 2111, to rotate by 90° or more. That is, the coupling member 323, which is a coupling part of the wire 301 and the pulley 2111, is rotatable until being located on a common internal tangent of the pulley 2111 and the pulley 2112, thereby increasing the range of rotation.

In other words, the wire 305 is located on the internal tangent of the pulley 2111 and the pulley 2112, and the rotation angle of the pulley 2111 is increased by the pulley 2112.

Next, components related to the rotation of the pulley 2121 will be described.

The pulley 2123 is disposed on one side of the pulley 2121,. Here, the pulley 2123 is formed to be rotatable around the rotation shaft 2143, which is an end tool pitch rotation shaft. In addition, the pulley 2125 is disposed on one side of the pulley 2123. Here, the pulley 2125 is formed to be rotatable around the rotation shaft 2144, which is an end tool pitch auxiliary rotation shaft. Here, in the drawings, it is illustrated that all of the pulley 2123 and the pulley 2125 are formed to be rotatable around the Y-axis direction, but the concept of the present disclosure is not limited thereto, and the rotation shafts of the respective pulleys may be formed in various directions according to configurations thereof.

The retraction wire 302 may be sequentially wound to make contact with at least portions of the pulley 2121, the pulley 2122, the pulley 2123, and the pulley 2125.

Accordingly, when the wire 302 is pulled toward an arrow 302 of FIG. 9, the wire 302 may provide a driving force to cause the operation member 540 to move backward.

Hereinafter, a pitch motion of the present disclosure will be described in more detail.

Meanwhile, when the wire 301 is pulled toward the arrow 301 of FIG. 7, and simultaneously, the wire 305 is pulled toward the arrow 305 of FIG. 7 (that is, when both strands of the first jaw wire are pulled), since the wires 301 and 305 are wound around lower portions of the pulley 2113 and the pulley 2114 rotatable around the rotation shaft 2143, which is an end tool pitch rotation shaft, the pulley 2111 to which the wires 301 and 305 are fixedly coupled and the end tool hub 2106 to which the pulley 2111 is coupled are rotated as a whole in the counterclockwise direction around the rotation shaft 2143, so that the end tool 2100 is rotated downward to perform a pitch motion.

Meanwhile, the end tool 2100 of the surgical instrument 2000 of the present disclosure may further include the pulley 2131, which is an end tool pitch pulley, the manipulation part 200 may further include one or more pulleys (not shown), which are manipulation part pitch pulleys, and the power transmission part 300 may further include the wires 303 and 304, which are pitch wires. In detail, the pulley 2131 of the end tool 2100 is rotatable around the rotation shaft 2143, which is an end tool pitch rotation shaft, and may be integrally formed with the end tool hub 2106 (or fixedly coupled to the end tool hub 2106) as one body. In addition, one or more pitch wires may connect the pulley 2131 of the end tool 2100 to the pulley of the manipulation part 200.

In addition, the wires 303 and 304 may serve to connect the pulley 2131 of the end tool 2100 to the pulleys (not shown) of the manipulation part 200 and a pulley (not shown) different therefrom.

Accordingly, when the pulley of the manipulation part 200 rotates, this rotation is transmitted through the wires to the pulley 2131 of the end tool 2100, causing the pulley 2131 to rotate as well, and as a result, the end tool 2100 performs a pitch motion while rotating.

That is, in the surgical instrument 2000 according to the first embodiment of the present disclosure, by providing the pulley 2131 of the end tool 2100, one or more pulleys of the manipulation part 200 and the pulley 232, and one or more wires 303 and 304 of the power transmission part 300 to transmit a driving force for a pitch motion, the driving force for the pitch motion of the manipulation part 200 may be more completely transmitted to the end tool 2100, thereby improving operation reliability.

Here, a diameter of each of the pulley 2113 and the pulley 2114, which are end tool jaw pitch main pulleys, and a diameter of the pulley 2131, which is an end tool pitch pulley, may either be the same or different from each other. At this time, a ratio of the diameter of the end tool jaw pitch main pulley to the diameter of the end tool pitch pulley may be the same as a ratio of a diameter of the manipulation part pitch pulley of the manipulation part 200 to a diameter of a manipulation part pitch main pulley.

### (Components Related to Staple Pulley)

Hereinafter, the staple pulleys 2181 and 2191 of the end tool 2100 of the surgical instrument 2000 of FIG. 2 will be described in more detail.

FIG. 11 is a side view illustrating the end tool of the surgical instrument of FIG. 4. FIGS. 12 to 15 are exploded perspective views of the end tool of the surgical instrument of FIG. 4.

FIG. 16 is a side view illustrating the second jaw of the surgical instrument of FIG. 4, and FIG. 17 is a cross-sectional view illustrating the second jaw of the surgical instrument of FIG. 4. FIG. 18 is a side view illustrating the first jaw of the surgical instrument of FIG. 4, and FIG. 19 is a cross-sectional view illustrating the first jaw of the surgical instrument of FIG. 4.

FIG. 20 is a cross-sectional view illustrating an open state of the first and second jaws of the surgical instrument of FIG. 4, and FIG. 21 is a cross-sectional view illustrating a closed state of the first and second jaws of the surgical instrument of FIG. 4.

FIGS. 22 and 23 are exploded perspective views illustrating the staple drive assembly of the surgical instrument of FIG. 4.

FIGS. 24 and 25 are side views illustrating an operation of the staple drive assembly of the surgical instrument of FIG. 4.

FIGS. 26 and 27 are perspective views illustrating operations of the staple drive assembly of the surgical instrument of FIG. 4.

Referring to FIGS. 4 to 27 and the like, the end tool 2100 of the first embodiment of the present disclosure may include the first staple pulley 2181, a first staple auxiliary pulley 2182, a pulley 2183, a pulley 2184, a pulley 2185, and a pulley 2186 that are related to a linear motion/rotational motion of respective pulleys and links for stapling and cutting. In addition, the end tool 2100 of the first embodiment of the present disclosure may further include the pulley 2187 and the pulley 2188. In addition, the end tool 2100 of the first embodiment of the present disclosure may include the second staple pulley 2191, a second staple auxiliary pulley 2192, a pulley 2193, a pulley 2194, a pulley 2195, and a pulley 2196 that are related to a linear motion/rotational motion of respective pulleys and links for stapling and cutting. In addition, the end tool 2100 of the first embodiment of the present disclosure may further include the pulley 2197 and the pulley 2198. The first staple pulley 2181 and the second staple pulley 2191 are formed to respectively face the pulley 2121, which is a pulley for the retraction wire, and the pulley 2111, which is an end tool jaw pulley, and formed to be rotatable independently of each other around the rotation shaft 2141, which is an end tool jaw pulley rotation shaft. Here, in the drawing, it is illustrated that the first staple pulley 2181 and the second staple pulley 2191 are located between the pulley 2111 and the pulley 2121, but the concept of the present disclosure is not limited thereto, and the first staple pulley 2181 and the second staple pulley 2191 may be located at various positions adjacent to the pulley 2111 or the pulley 2121.

Here, in the present disclosure, the first staple pulley 2181, the second staple pulley 2191, the pulley 2111, and the pulley 2121 are formed to rotate around substantially the same shaft. As described above, as the first staple pulley 2181, the second staple pulley 2191, the pulley 2111, and the pulley 2121 are formed to rotate around the same shaft, it is possible to perform a pitch motion/yaw motion/actuation motion as well as stapling and cutting motions. This will be described in more detail later. However, here, although it is illustrated in the drawing that the first staple pulley 2181, the second staple pulley 2191, the pulley 2111, and the pulley 2121 are formed to rotate around one rotation shaft 2141, it is of course possible that each pulley may be formed to be rotatable around a separate shaft that is concentric therewith.

In other words, it may be expressed that a structure is provided in which the pulley 2111, which is a first jaw pulley, the first staple pulley 2181, the second staple pulley 2191, and the pulley 2121, which is a pulley for the retraction wire, are sequentially stacked along the rotation shaft 2141. Alternatively, it may be expressed that a structure is provided in which the first staple pulley 2181 and the second staple pulley 2191 are disposed between pulleys 2111 and 2121 facing each other. Here, the pulley 2111, which is a first jaw pulley, the first staple pulley 2181, the second staple pulley 2191, and the pulley 2121, which is a pulley for the retraction wire, may be formed to be rotatable independently of each other.

The first staple auxiliary pulley 2182 may be additionally provided on one side of the first staple pulley 2181, in other words, the first staple auxiliary pulley 2182 may be disposed between the first staple pulley 2181 and the pulley 2183/pulley 2184. The first staple auxiliary pulley 2182 may be formed to be rotatable independently of the pulley 2112 and the pulley 2122 around the rotation shaft 2142.

Meanwhile, the pulley 2187 and the pulley 2188 may be additionally disposed between the first staple auxiliary pulley 2182 and the pulley 2183/pulley 2184. The pulley 2187 and the pulley 2188 may be formed to be rotatable around the separation-prevention-pulley coupling part 2106f of the end tool hub 2106. Here, the separation-prevention-pulley coupling part 2106f may be formed to be parallel to the rotation shaft 2143, which is a central axis of the pulley 2183 and the pulley 2184. Here, the pulley 2187 and the pulley 2188 may function as first-staple-wire-separation-prevention pulleys.

Meanwhile, the pulleys 2183 and 2184 may function as staple pitch main pulleys, and the pulleys 2185 and 2186 may function as staple pitch sub-pulleys.

The second staple auxiliary pulley 2192 may be additionally provided on one side of the second staple pulley 2191, in other words, the second staple auxiliary pulley 2192 may be disposed between the second staple pulley 2191 and the pulley 2193/pulley 2194. The second staple auxiliary pulley 2192 may be formed to be rotatable independently of the pulley 2112 and the pulley 2122 around the rotation shaft 2142.

Here, it is illustrated in the drawing that the first staple auxiliary pulley 2182, the second staple auxiliary pulley 2192, the pulley 2112, and the pulley 2122 are formed to rotate around one rotation shaft 2142, but it is of course possible that each of the first staple auxiliary pulley 2182, the second staple auxiliary pulley 2192, the pulley 2112, and the pulley 2122 may be formed to be rotatable around a separate shaft. Such staple auxiliary pulleys will be described in more detail later.

Meanwhile, the pulley 2197 and the pulley 2198 may be additionally disposed between the second staple auxiliary pulley 2192 and the pulley 2193/pulley 2194. The pulley 2197 and the pulley 2198 may be formed to be rotatable around the separation-prevention-pulley coupling part 2106f of the end tool hub 2106. Here, the separation-prevention-pulley coupling part 2106f may be formed to be parallel to the rotation shaft 2143, which is the central axis of the pulley 2183 and the pulley 2184. Here, the pulley 2197 and the pulley 2198 may function as second staple wire separation-prevention pulleys.

Meanwhile, the pulley 2193 and the pulley 2194 may function as staple pitch main pulleys, and the pulley 2195 and the pulley 2196 may function as staple pitch sub-pulleys.

Hereinafter, the first staple auxiliary pulley 2182 will be described in more detail.

The first staple auxiliary pulley 2182 may serve to increase a rotation angle of the first staple pulley 2181 by coming into contact with the wire 308, which is a first staple wire, to change an arrangement path of the wire 308 to a certain extent.

That is, when the staple auxiliary pulley is not disposed, the staple pulley may be rotated only up to a right angle, but in an embodiment of the present disclosure, by additionally providing the first staple auxiliary pulley 2182, which is an auxiliary pulley, the maximum rotation angle may be increased by θ. This allows the first staple pulley 2181 to rotate for the stapling and cutting motions while two jaws of the end tool 2100 are yaw-rotated together by 90°, thereby enabling a linear motion of the operation member 540 to be described later. In other words, a feature of increasing the range of yaw rotation in which stapling and cutting motions are possible may be obtained through the first staple auxiliary pulley 2182.

This will be described below in more detail.

In the case of the surgical instrument 2000 of the present disclosure, the first staple auxiliary pulley 2182 is further disposed on one side of the first staple pulley 2181. By changing the arrangement path of the wire 308, which is a first staple wire, to a certain extent by disposing the first staple auxiliary pulley 2182 as described above, a tangential direction of the wire 308 is changed, and thus the rotation angle of the coupling member (see 329 of FIG. 56) that couple the wire 308 to the first staple pulley 2181 is increased. That is, the coupling member (see 329 of FIG. 56), which is a coupling part of the wire 308 and the first staple pulley 2181, is rotatable until the coupling member (see 329 of FIG. 56) is located on a common internal tangent of the first staple pulley 2181 and the first staple auxiliary pulley 2122.

In other words, the wire 308 is located on the internal tangent of the first staple pulley 2181 and the first staple auxiliary pulley 2182, and the rotation angle of the first staple pulley 2181 is increased by the first staple auxiliary pulley 2182.

According to the present disclosure, as a rotation radius of the first staple pulley 2181 increases, a yaw motion range in which normal stapling and cutting motions are performed may be increased.

Hereinafter, the pulley 2187 and the pulley 2188, which are first-staple-wire-separation-prevention pulleys, will be described in more detail.

In the end tool 2100 of the surgical instrument according to the first embodiment of the present disclosure, the pulleys 2187 and 2188, which are first-staple-wire-separation-prevention pulleys, are additionally provided to serve to prevent the wires 307 and 308, which are first staple wires, from being separated.

That is, by arranging the pulley 2187/pulley 2188 between the first staple auxiliary pulley 2182 and the pulley 2183/pulley 2184, the path of the wire 307 passing through the pulley 2183 and heading toward the first staple pulley 2181 and the path of the wire 308 passing through the pulley 2184 and heading toward the first staple auxiliary pulley 2182 are changed to a certain extent. More specifically, the paths of the wire 307/wire 308 are changed to a certain extent so that the wire 307 heading toward the first staple pulley 2181 through the pulley 2183 and the wire 308 heading toward the first staple auxiliary pulley 2182 through the pulley 2184 become parallel to the X-axis.

In detail, a Z-axis height of the wire 307 coming out of the pulley 2183 and the Z-axis height of the wire 307 heading toward the first staple pulley 2181 are different from each other. Similarly, a Z-axis height of the wire 308 coming out the pulley 2184 and the Z-axis height of the wire 308 heading toward the first staple auxiliary pulley 2182 are different from each other. Accordingly, in the absence of pulley 2187/pulley 2188, which are first-staple-wire-separation-prevention pulleys, the paths the wire 307/wire 308 become diagonal (i.e., the fleet angle of the wire relative to the pulley increases), and thus, there is a risk of the wire 307/wire 308 separating from the pulleys, and a risk of the wire 307/wire 308 being damaged.

Thus, in the present embodiment, the pulley 2187/pulley 2188, which are first-staple-wire-separation-prevention pulleys, are disposed between the first staple auxiliary pulley 2182 and the pulley 2183/pulley 2184, and serve to change the paths of the wire 307/wire 308, which head toward the distal end 2104 of the end tool 2100 after being wound around the pulley 2183/pulley 2184, to a certain extent so that wire 307/wire 308 become parallel to the X-axis.

According to the present disclosure, the wires 307 and 308, which are first staple wires, are prevented from being separated from the pulleys, thereby more smoothly performing the cutting motion. Hereinafter, components related to the rotation of the first staple pulley 2181 will be described.

The pulleys 2183 and 2184 function as staple pitch main pulleys. Here, the wire 307, which is a first staple wire, is wound around the pulley 2183, and the wire 308, which is a first staple wire, is wound around the pulley 2184.

The pulleys 2185 and 2186 function as staple pitch sub-pulleys. Here, the wire 307, which is a first staple wire, is wound around the pulley 2185, and the wire 308, which is a first staple wire, is wound around the pulley 2186.

Here, the pulley 2183 and the pulley 2184 are disposed on one side of the first staple pulley 2181, the first staple auxiliary pulley 2182, and the pulley 2187/pulley 2188 so as to face each other. Here, the pulley 2183 and the pulley 2184 are formed to be rotatable independently of each other around the rotation shaft 2143 that is an end tool pitch rotation shaft. In addition, the pulley 2185 and the pulley 2186 are disposed on one side of the pulley 2183 and one side of the pulley 2184, respectively, to face each other. Here, the pulley 2185 and the pulley 2186 are formed to be rotatable independently of each other around the rotation shaft 2144 that is an end tool pitch auxiliary rotation shaft. Here, in the drawings, it is illustrated that the pulley 2183, the pulley 2185, the pulley 2184, and the pulley 2186 are all formed to be rotatable around the Y-axis direction, but the concept of the present disclosure is not limited thereto, and the rotation shafts of the respective pulleys may be formed in various directions according to configurations thereof.

As described above, the rotation shaft 2141, the rotation shaft 2142, the rotation shaft 2143, and the rotation shaft 2144 may be arranged sequentially from the distal end 2104 toward the proximal end 2105 of the end tool 2100. Accordingly, the first staple pulley 2181, the first staple auxiliary pulley 2182, the pulley 2187/pulley 2188, the pulley 2183/pulley 2184, and the pulley 2185/pulley 2186 may be arranged sequentially from the distal end 2104 toward the proximal end 2105 of the end tool 2100.

The wire 307, which is a first staple wire, is sequentially wound to make contact with at least portions of the pulley 2185, the pulley 2183, the pulley 2187, and the first staple pulley 2181. In addition, the wire 308 connected to the wire 307 by the coupling member (see 329 of FIG. 56) is sequentially wound to make contact with at least portions of the first staple pulley 2181, the first staple auxiliary pulley 2182, the pulley 2188, the pulley 2184, and the pulley 2186.

In other words, the wires 307 and 308, which are first staple wires, are sequentially wound to make contact with at least portions of the pulley 2185, the pulley 2183, the pulley 2187, the first staple pulley 2181, the first staple auxiliary pulley 2182, the pulley 2188, the pulley 2184, and the pulley 2186, and are formed to move along the above pulleys while rotating the above pulleys.

Accordingly, when the wire 307 is pulled, the coupling member (see 329 of FIG. 56) to which the wire 307 is coupled and the first staple pulley 2181 coupled thereto are rotated in one direction. In contrast, when the wire 308 is pulled, the coupling member (see 329 of FIG. 56) to which the wire 308 is coupled and the first staple pulley 2181 coupled thereto are rotated in a direction opposite to the one direction.

Meanwhile, the second staple pulley 2191 and the second staple auxiliary pulley 2192, and components related thereto, such as the pulley 2193, the pulley 2194, the pulley 2195, the pulley 2196, the pulley 2197, the pulley 2198, the wire 309, and the wire 310, may have the same or similar configurations as the components related to the first staple pulley 2181 described above.

In detail, the pulleys 2193 and 2194 function as staple pitch main pulleys. Here, the wire 310, which is a second staple wire, is wound around the pulley 2193, and the wire 309, which is a second staple wire, is wound around the pulley 2194.

The pulleys 2195 and 2196 function as staple pitch sub-pulleys. Here, the wire 310, which is a second staple wire, is wound around the pulley 2195, and the wire 309, which is a second staple wire, is wound around the pulley 2196.

Here, the pulley 2193 and the pulley 2194 are disposed on one side of the second staple pulley 2191, the second staple auxiliary pulley 2192, and the pulley 2197/pulley 2198 so as to face each other. Here, the pulley 2193 and the pulley 2194 are formed to be rotatable independently of each other around the rotation shaft 2143 that is an end tool pitch rotation shaft. In addition, the pulley 2195 and the pulley 2196 are disposed on one side of the pulley 2193 and one side of the pulley 2194, respectively, to face each other. Here, the pulley 2195 and the pulley 2196 are formed to be rotatable independently of each other around the rotation shaft 2144 that is an end tool pitch auxiliary rotation shaft. Here, in the drawings, it is illustrated that the pulley 2193, the pulley 2195, the pulley 2194, and the pulley 2196 are all formed to be rotatable around the Y-axis direction, but the concept of the present disclosure is not limited thereto, and the rotation shafts of the respective pulleys may be formed in various directions according to configurations thereof.

As described above, the rotation shaft 2141, the rotation shaft 2142, the rotation shaft 2143, and the rotation shaft 2144 may be arranged sequentially from the distal end 2104 toward the proximal end 2105 of the end tool 2100. Accordingly, the second staple pulley 2191, the second staple auxiliary pulley 2192, the pulley 2197/pulley 2198, the pulley 2193/pulley 2194, and the pulley 2195/pulley 2196 may be arranged sequentially from the distal end 2104 toward the proximal end 2105 of the end tool 2100.

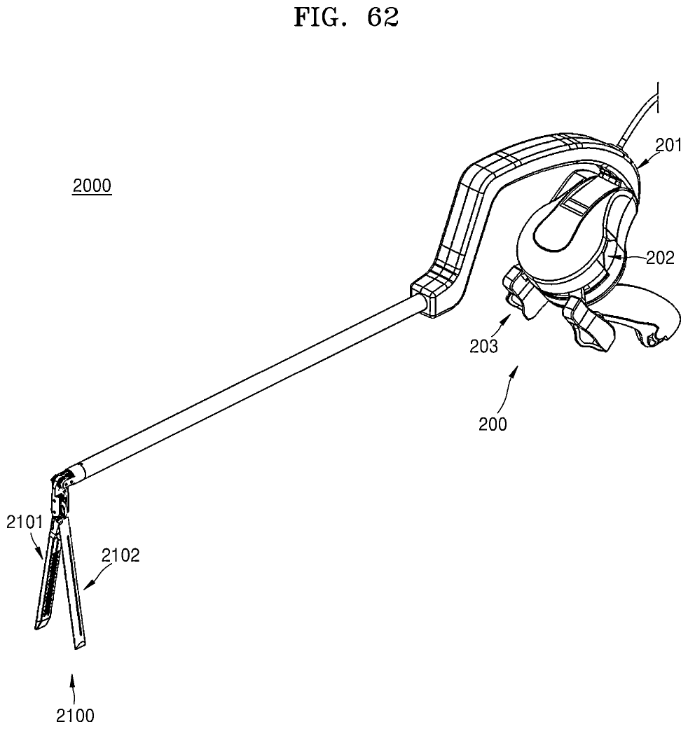

The wire 310, which is a second staple wire, is sequentially wound to make contact with at least portions of the pulley 2195, the pulley 2193, the pulley 2197, and the second staple pulley 2191. In addition, the wire 309 connected to the wire 310 by the coupling member (see 330 of FIG. 62) is sequentially wound to make contact with at least portions of the second staple pulley 2191, the second staple auxiliary pulley 2192, the pulley 2198, the pulley 2194, and the pulley 2196.

In other words, the wire 310 and the wire 309, which are second staple wires, are sequentially wound to make contact with at least portions of the pulley 2195, the pulley 2193, the pulley 2197, the second staple pulley 2191, the second staple auxiliary pulley 2192, the pulley 2198, the pulley 2194, and the pulley 2196, and are formed to move along the above pulleys while rotating the above pulleys.

Accordingly, when the wire 310 is pulled, the coupling member (see 330 of FIG. 57) to which the wire 310 is coupled and the second staple pulley 2191 coupled thereto are rotated in one direction. In contrast, when the wire 309 is pulled, the coupling member (see 330 of FIG. 57) to which the wire 309 is coupled and the second staple pulley 2191 coupled thereto are rotated in a direction opposite to the one direction.

### (Staple Drive Assembly)

Hereinafter, a staple drive assembly 2150 will be described in more detail.

Referring to FIGS. 12 to 27 and the like, the staple drive assembly 2150 may include a staple pulley assembly 2160 and the staple link assembly 2170. Here, the staple drive assembly 2150 is connected to a reciprocating assembly 550 of a cartridge 500, which will be described later, and converts a rotational motion of the staple pulley assembly 2160 into a linear motion of the reciprocating assembly 550. In other embodiments of the present disclosure, which will be described later, the staple drive assembly may be understood as a concept including the staple pulley assembly and the staple link assembly.

The staple pulley assembly 2160 may include one or more staple pulleys. The staple pulley assembly 2160 may be formed between the pulley 2111 and the pulley 2121 to be adjacent to the pulley 2111 and the pulley 2121. In an embodiment, it is assumed that the staple pulley assembly 2160 includes two pulleys that are the first staple pulley 2181 and the second staple pulley 2191.

The staple link assembly 2170 may include one or more link members 2171. In addition, the link member 2171 may include one or more links. In the first embodiment of the present disclosure, it is assumed that the staple link assembly 2170 includes one link member 2171, and the link member 2171 includes one link.

In the end tool 2100 of the surgical instrument according to the present disclosure, the staple pulley assembly 2160 and the staple link assembly 2170 form a cam-slot structure. In addition, such a structure provides the effect of amplifying a force for moving the reciprocating assembly 550 forward.

In detail, the staple pulley assembly 2160 may include the first staple pulley 2181 and the second staple pulley 2191.

The first staple pulley 2181 may include a body 2181a, a protruding member 2181b, and a shaft pass-through part 2181c.

The body 2181a is formed in the shape of a disk.

The shaft pass-through part 2181c may be formed in a center portion of the body 2181a. The shaft pass-through part 2181c may be formed in the shape of a hole, and the rotation shaft 2141, which is an end tool jaw pulley rotation shaft, may be inserted through the shaft pass-through part 2181c.

In addition, the protruding member 2181b may be formed on the body 2181a of the first staple pulley 2181. The protruding member 2181b may be coupled to the link member 2171 of the staple link assembly 2170. Here, the center of the protruding member 2181b may not coincide with the center of the first staple pulley 2181, and the protruding member 2181b may be formed to be eccentric to a certain extent with respect to the first staple pulley 2181. The protruding member 2181b may be fitted into a first slot 2171d of the link member 2171, which will be described later.

The second staple pulley 2191 may include a body 2191a, a protruding member 2191b, and a shaft pass-through part 2191c.

The body 2191a is formed in the shape of a disk.

The shaft pass-through part 2191c may be formed in a center portion of the body 2191a. The shaft pass-through part 2191c may be formed in the shape of a hole, and the rotation shaft 2141, which is an end tool jaw pulley rotation shaft, may be inserted through the shaft pass-through part 2191c.

In addition, the protruding member 2191b may be formed on the body 2191a of the second staple pulley 2191. The protruding member 2191b may be coupled to the link member 2171 of the staple link assembly 2170. Here, the center of the protruding member 2191b may not coincide with the center of the second staple pulley 2191, and the protruding member 2191b may be formed to be eccentric to a certain extent with respect to the second staple pulley 2191. The protruding member 2191b may be fitted into a second slot 2171e of the link member 2171, which will be described below.

Meanwhile, the end tool 2100 of the present disclosure may further include the staple link assembly 2170 connected to the staple pulley assembly 2160, and the staple link assembly 2170 may include the link member 2171. Here, the staple link assembly 2170 may serve to connect the staple pulley assembly 2160 to the reciprocating assembly 550 of the cartridge 500, which will be described later.

In an embodiment, the staple link assembly 2170 includes one link member 2171, and the link member 2171 includes only one link. That is, by coupling the staple pulley assembly 2160 and the staple link assembly 2170 using a cam-slot structure, a rotational motion of the staple pulley assembly 2160 can be converted into a linear motion of the staple link assembly 2170 even when the staple link assembly 2170 includes only one link.

In detail, the link member 2171 may be formed as a single link.

The link member 2171 is formed in a shape in which an elongated bar and an elliptical-shaped flat plate are coupled, and may have a bent part, for example, formed approximately in the shape of the alphabet letter “L.” Here, the link member 2171 may include a first protrusion 2171a, a second protrusion 2171b, a coupling part 2171c, the first slot 2171d, and the second slot 2171e.

The first protrusion 2171a and the second protrusion 2171b may be formed in one region of a central portion of the link member 2171. The first protrusion 2171a and the second protrusion 2171b may be fitted into a guide groove 2101c of the first jaw 2101.

As described above, as the first protrusion 2171a and the second protrusion 2171b are moved along the guide groove 2101c in a state in which the first protrusion 2171a and the second protrusion 2171b of the link member 2171 formed in a protruding shape are fitted into the groove-shaped guide groove 2101c, the link member 2171 is moved with respect to the first jaw 2101 (and the cartridge 500 therein). This will be described in more detail later.

Meanwhile, the coupling part 2171c may be formed at one end portion of the link member 2171. The coupling part 2171c may be coupled to a coupling part 551a of a reciprocating member 551 of the cartridge 500.

Meanwhile, the first slot 2171d and the second slot 2171e may be formed at an end portion of the link member 2171 opposite to the one end portion at which the coupling part 2171c is formed.

In detail, the first slot 2171d may be formed in a surface of the link member 2171 facing the first staple pulley 2181. Here, the first slot 2171d is formed in the shape of an elongated hole, into which the protruding member 2181b of the first staple pulley 2181 may be fitted. The first slot 2171d is formed to have a certain curvature, and may be formed in an approximately elliptical shape. At this time, the first slot 2171d may be formed to be larger than the protruding member 2181b by a certain extent. Thus, the protruding member 2181b is formed to be movable in the first slot 2171d by a certain extent in a state in which the protruding member 2181b of the first staple pulley 2181 is fitted into the first slot 2171d of the link member 2171.

As described above, the protruding member 2181b may be formed to be eccentric to a certain extent with respect to the center of the first staple pulley 2181. Thus, when the first staple pulley 2181 is rotated, the protruding member 2181b, while in contact with the first slot 2171d, may push the first slot 2171d to move the link member 2171. That is, when the first staple pulley 2181 is rotated, the protruding member 2181b, while in contact with the first slot 2171d, is moved in the first slot 2171d, which causes the link member 2171 to be linearly moved along the guide groove 2101c of the first jaw 2101.

Here, the first slot 2171d may be formed not to pass through the entire thickness of the link member 2171 but to pass through approximately half of the entire thickness of the link member 2171. In other words, the first slot 2171d may be formed to have substantially the same thickness as the thickness of the protruding member 2181b of the first staple pulley 2181.

Meanwhile, the second slot 2171e may be formed in the link member 2171. In detail, the second slot 2171e may be formed in a surface of the link member 2171 facing the second staple pulley 2191. Here, the second slot 2171e is formed in the shape of an elongated hole, into which the protruding member 2191b of the second staple pulley 2191 may be fitted. The second slot 2171e is formed to have a certain curvature, and may be formed in an approximately elliptical shape. At this time, the second slot 2171e may be formed to be larger than the protruding member 2191b by a certain extent. Thus, the protruding member 2191b is formed to be movable in the second slot 2171e by a certain extent in a state in which the protruding member 2191b of the second staple pulley 2191 is fitted into the second slot 2171e of the link member 2171.

As described above, the protruding member 2191b may be formed to be eccentric to a certain extent with respect to the center of the second staple pulley 2191. Thus, when the second staple pulley 2191 is rotated, the protruding member 2191b, while in contact with the second slot 2171e, may push the second slot 2171e to move the link member 2171. That is, when the second staple pulley 2191 is rotated, the protruding member 2191b, while in contact with the second slot 2171e, is moved in the second slot 2171e, which causes the link member 2171 to be linearly moved along the guide groove 2101c of the first jaw 2101.

Here, the second slot 2171e may be formed not to pass through the entire thickness of the link member 2171 but to pass through approximately half of the entire thickness of the link member 2171. In other words, the second slot 2171e may be formed to have substantially the same thickness as the thickness of the protruding member 2191b of the second staple pulley 2191.

Here, the first slot 2171d and the second slot 2171e may be formed to at least partially overlap. In addition, the sum of the thicknesses of the first slot 2171d and the second slot 2171e in the Y-axis direction may be formed to be approximately equal to the thickness of the link member 2171 in the Y-axis direction.

Here, the first slot 2171d and the second slot 2171e may be formed to be vertically symmetrical with respect to the rotation shaft 2141. As such, since the first slot 2171d and the second slot 2171e are formed to be vertically symmetrical with respect to the rotation shaft 2141, the protruding member 2181b of the first staple pulley 2181 coupled to the link member 2171 and the protruding member 2191b of the second staple pulley 2191 may be disposed to be symmetrical to each other. This will be described in more detail later.

### (Displacement and Motion of Staple Link Assembly According to Rotation of Staple Pulley)

Hereinafter, a displacement of the staple link assembly 2170 according to the rotation of the first staple pulley 2181 and the second staple pulley 2191 will be described.

Referring to FIG. 24, in the first embodiment of the present disclosure, the first staple pulley 2181 and the staple link assembly 2170 are coupled in the form of a cam-slot. That is, the cam-shaped protruding member 2181b formed on the first staple pulley 2181 is coupled to the first slot 2171d formed in the link member 2171. Accordingly, when the first staple pulley 2181 is rotated in the direction of an arrow A, a displacement of the protruding member 2181b of the first staple pulley 2181 in the X-axis direction becomes B. In addition, a displacement of the staple link assembly 2170 in the X-axis direction becomes C.

Similarly, referring to FIG. 25, in the first embodiment of the present disclosure, the second staple pulley 2191 and the staple link assembly 2170 are coupled to each other in a cam-slot configuration. That is, the cam-shaped protruding member 2191b formed on the second staple pulley 2191 is coupled to the second slot 2171e formed in the link member 2171. Accordingly, when the second staple pulley 2191 is rotated in the direction of an arrow D, a displacement of the protruding member 2191b of the second staple pulley 2191 in the X-axis direction becomes E. In addition, the displacement of the staple link assembly 2170 in the X-axis direction becomes F.

When the staple pulley and the staple link assembly are coupled in a link-shaft manner rather than a cam-slot manner as compared with the above case, the displacement of the staple link assembly in the X-axis direction will be much longer than that in the first embodiment of the present disclosure.

In other words, as compared to the case in which the staple pulley and the staple link assembly are axially coupled, when the staple pulley and the staple link assembly are cam-slot coupled as in the present embodiment, the displacement of the staple link assembly in the X-axis direction will be reduced even when the staple pulley is rotated by the same amount.

Meanwhile, since work is the product of force and displacement, assuming that the work for rotating the staple pulley is the same, the displacement and the force are inversely proportional to each other. Accordingly, when the displacement is reduced, the force is increased in inverse proportion to the displacement.

As a result, in the first embodiment of the present disclosure, each of the first staple pulley 2181 and the second staple pulley 2191 is coupled in a cam-slot configuration to the staple link assembly 2170, and the displacement of the staple link assembly 2170 in the X-axis direction caused by the rotation of the first staple pulley 2181 and the second staple pulley 2191 is relatively reduced as compared to other embodiments, and thus the force received by the staple link assembly 2170 in the X-axis direction is increased relative to a simple link structure.

According to the first embodiment of the present disclosure described above, a force for moving the staple link assembly 2170 and the reciprocating assembly 550 connected thereto forward is amplified, thereby achieving the effect of performing a stapling motion more robustly.

In particular, in the first embodiment of the present disclosure, since two staple pulleys (i.e., the first staple pulley 2181 and the second staple pulley 2191) that are symmetrical to each other are provided, a force with which the staple pulley assembly 2160 pushes the staple link assembly 2170 may be amplified approximately twice as compared to when only one staple pulley is provided.

In addition, the first staple pulley 2181 and the second staple pulley 2191 are symmetrically disposed in a left and right direction with respect to the XZ plane, and thus balanced in the left and right direction in performing a stapling motion, so that the end tool 2100 stably performs motions with respect to the rotation shaft 2141, which is a yaw rotation shaft, without moving in the left and right direction as a whole. In addition, by making winding directions of the wire 307/wire 308, which are first staple wires, and the second staple wire 309/wire 310 around the rotation shaft 2143, which is a pitch rotation shaft, to be opposite to each other, movements with respect to the rotation shaft 2143 may also be mutually canceled.

Hereinafter, rotation directions of the first staple pulley 2181 and the second staple pulley 2191 will be described.

Referring to FIGS. 24 to 27 and the like, the first staple pulley 2181 moves the staple link assembly 2170 forward when rotated in the direction of an arrow A of FIG. 27 (i.e., in the clockwise direction), and the second staple pulley 2191 moves the staple link assembly 2170 forward when rotated in the direction of an arrow D of FIG. 27 (i.e., in the counterclockwise direction).

In contrast, the first staple pulley 2181 moves the staple link assembly 2170 backward when rotated in the counterclockwise direction, and the second staple pulley 2191 moves the staple link assembly 2170 backward when rotated in the clockwise direction.

As a result, when the first staple pulley 2181 and the second staple pulley 2191 are rotated in opposite directions, the staple link assembly 2170 is moved (forward or backward). In contrast, when the first staple pulley 2181 and the second staple pulley 2191 are rotated in the same direction, the rotations of the two pulleys are canceled out and thus the staple link assembly 2170 is not moved.

As a result, in the state as shown in FIG. 26, when the first staple pulley 2181 is rotated in the clockwise direction while the second staple pulley 2191 is rotated in the counterclockwise direction, the link member 2171 connected to the first staple pulley 2181 and the second staple pulley 2191 may be moved as a whole toward the distal end of the first jaw 2101 (in the rightward direction of FIG. 20).

In contrast, when the first staple pulley 2181 is rotated in the counterclockwise direction while the second staple pulley 2191 is rotated in the clockwise direction, the link member 2171 connected to the first staple pulley 2181 and the second staple pulley 2191 may be moved as a whole toward the proximal end of the first jaw 2101 (in the leftward direction of FIG. 20).

Thus, a bidirectional rotational movement of the staple pulley assembly 2160 causes a reciprocating linear movement of the reciprocating assembly 550 of the cartridge 500 through the staple link assembly 2170. This will be described in more detail later.

### (First and Second Jaws and Actuation Motion)

Hereinafter, a coupling structure of the first jaw 2101 and the second jaw 2102 of the end tool 2100 of the surgical instrument 2000 of FIG. 2 will be described in more detail.

FIGS. 12 to 15 are exploded perspective views of the end tool of the surgical instrument of FIG. 4.

FIG. 16 is a side view illustrating the second jaw of the surgical instrument of FIG. 4, and FIG. 17 is a cross-sectional view illustrating the second jaw of the surgical instrument of FIG. 4. FIG. 18 is a side view illustrating the first jaw of the surgical instrument of FIG. 4, and FIG. 19 is a cross-sectional view illustrating the first jaw of the surgical instrument of FIG. 4.

FIGS. 20 and 21 are cross-sectional views illustrating opening and closing motions of the first jaw and the second jaw of the surgical instrument of FIG. 4.

FIG. 46 is a view illustrating a process in which the operation member is coupled to the second jaw.

Referring to FIGS. 2 to 21 and the like, the first jaw 2101 may include a first jaw body 2101a, a cartridge accommodation part 2101b, the guide groove 2101c, a shaft coupling part 2101d, a first jaw coupling part 2101e, and a staple assembly accommodation part 2101f.

The first jaw 2101 is formed in an overall elongated bar shape, and, for example, the first jaw body 2101a may have a shape similar to an elongated bar. The cartridge 500 is accommodated in the first jaw 2101 at the distal end side (see 2104 of FIG. 4), and the pulley 2111 is coupled to the proximal end side (see 2105 of FIG. 4) and formed to be rotatable around the rotation shaft 2141. In other words, the first jaw 2101 may be formed entirely in the shape of a hollow box with one surface (upper surface) thereof is removed, such that the cartridge accommodation part 2101b capable of accommodating the cartridge 500 may be formed inside the first jaw 2101. That is, the first jaw 2101 may be formed in an approximately “U” shape in cross section.

Here, the first jaw 2101 and the pulley 2111 may be integrally formed, or may be formed as separate members and then coupled to each other.

The guide groove 2101c configured to guide the movement of the staple link assembly 2170, which will be described later, may be formed on one side of the cartridge accommodation part 2101b of the first jaw 2101, e.g., on the side of the proximal end (see 2105 of FIG. 4). The guide groove 2101c may be formed in the shape of a groove formed along a moving path of the staple link assembly 2170. In addition, as the first protrusion 2171a and the second protrusion 2171b are moved along the guide groove 2101c in a state in which the first protrusion 2171a and the second protrusion 2171b of the link member 2171 formed in a protruding shape are fitted into the groove-shaped guide groove 2101c, the staple link assembly 2170 is moved with respect to the first jaw 2101 (and the cartridge 500 therein). That is, the staple link assembly 2170 may be moved along the guide groove 2101c of the first jaw 2101.

Meanwhile, the shaft coupling part 2101d, the first jaw coupling part 2101e, and the staple assembly accommodation part 2101f may be formed on the proximal end side of the first jaw 2101.

Here, the shafts of the pulley 2111 and the pulley 2121 may be coupled to the shaft coupling part 2101d. For example, the shaft coupling part 2101d may have a through-hole shape. For example, the shaft coupling part 2101d may be formed in the shape of a cylindrical hole. Accordingly, the pulley 2111 and the pulley 2121 may be formed to be rotatably coupled to the shaft coupling part 2101d of the first jaw 2101. This will be described in more detail below.

The first jaw coupling part 2101e may be formed relatively closer to the distal end (see 2104 of FIG. 4) of the first jaw 2101 than the shaft coupling part 2101d. The first jaw coupling part 2101e is formed in the shape of a hole, and the rotation shaft 2145 may correspond to the first jaw coupling part 2101e. The first jaw coupling part 2101e and a second jaw coupling part 2102b to be described later are axially coupled by the rotation shaft 2145, so that the second jaw 2102 can rotationally move relative to the first jaw 2101. Through this, the first jaw 2101 and the second jaw 2102 can perform opening and closing motions, such as an actuation motion.

The staple assembly accommodation part 2101f may be disposed closer to the proximal end of the body 2101a of the first jaw 2101 than the cartridge accommodation part 2101b. The staple assembly accommodation part 2101f may have a groove shape to accommodate one region of the second jaw 2102, for example, the second jaw coupling part 2102b. In addition, the staple assembly accommodation part 2101f may be formed to accommodate at least a portion of the staple drive assembly 2150 therein. For example, at least some of the first staple pulley 2181, the second staple pulley 2191, and the staple link assembly 2170 may be accommodated in the staple assembly accommodation part 2101f.

The second jaw 2102 may include a second jaw body 2102a, the second jaw coupling part 2102b, the guide part 2102c, and a passageway 2102d.

The second jaw 2102 is formed in an overall elongated bar shape, and, for example, the second jaw body 2102a may be formed in a bar shape to correspond to the first jaw 2101 in at least one region. An anvil is formed on the distal end (see 2104 of FIG. 4) side of the second jaw 21012, and the second jaw coupling part 2102b is formed on the proximal end (see 2105 of FIG. 4) and coupled to the first jaw 2101, thereby enabling the second jaw to rotate around the rotation shaft 2145.

In detail, among surfaces of the second jaw 2102, the surface facing the first jaw 2101 may have the anvil formed thereon, may be formed in a flat plane shape, and may have shapes corresponding to the shapes of staples 530 to be described below. The anvil of the second jaw 2102 may serve as a support for supporting the staple 530 on the opposite side of the operation member 540 when the operation member 540 pushes and raises the staple 530 during a stapling motion, so that the staple 530 is bent.

The guide part 2102c may be formed to guide the operation member 540 of the cartridge 500, and a groove may be formed in a region facing the operation member 540. In addition, the guide part 2102c may have a shape protruding toward the first jaw 2101 and a shape in which a groove is formed in the protruding region. The guide part 2102c may be formed, for example, between a region in which the anvil of the second jaw 2102 is formed and the second jaw coupling part 2102b. The operation member 540 of the cartridge 500 to be described later may be effectively guided through the guide part 2102c. The guide part 2102c may have an opening formed on one side thereof, for example, in a region facing the distal end and the first jaw 2101, and the opening may be connected to the passageway 2102d, which will be described later.

The passageway 2102d may be formed on an inner side of the second jaw 2102 to guide the movement of the operation member 540 of the cartridge 500 The passageway 2102d may be formed in the shape of a groove formed along a moving path of the operation member 540. In addition, the clamp 546 is moved along the passageway 2102d in a state in which the clamp 546 of the operation member 540 formed in a protruding shape is fitted into the groove-shaped passageway 2102d, thereby enabling the operation member 540 to move with respect to the second jaw 2102. That is, the operation member 540 may move along the passageway 2102d of the second jaw 2102, and as the clamp 546 is accommodated inside the second jaw 2102 through the movement of the operation member 540, a gap between the second jaw 2102 and the first jaw 2101 narrows, thereby enabling the second jaw 2102 to naturally maintain a closed state with respect to the first jaw 2101.

A coupling groove 2102i is formed in the distal end (the rightward direction of FIG. 17) of the second jaw 2102, and accordingly, the coupling groove 2102i and the opening of the guide part 2102c may be formed at both end portions of the passageway 2102d. The coupling groove 2102i and the opening of the guide part 2102c are formed to be slightly larger than the clamp 546 of the operation member 540, thereby allowing the clamp 546 to be drawn into the passageway 2102d of the second jaw 2102 or withdrawn from the passageway 2012d of the second jaw 2102 through the opening of the guide part 2102c. That is, the clamp 546 may be drawn into the second jaw 2102 through the opening of the guide part 2102c, moved along the passageway 2102d, and then withdrawn out of the second jaw 2102 through the coupling groove 2102i. This will be described in more detail below.

Meanwhile, the elastic member 2109 may be disposed between the first jaw 2101 and the second jaw 2102. The elastic member 2109 may be formed and disposed to apply a force in a direction in which the first jaw 2101 and the second jaw 2102 move away from each other. For example, the elastic member 2109 may have the form of a plate and a curved shape that branches outward toward both sides in the direction of the distal end of the end tool 2100.

The elastic member 2109 may be disposed adjacent to the first jaw coupling part 2101e and the second jaw coupling part 2102b. For example, at least one region of the elastic member 2109 may be accommodated in the staple assembly accommodation part 2101f of the first jaw 2101, while another region of the elastic member 2109 is accommodated in a groove adjacent to the second jaw coupling part 2102b of the second jaw 2102.

### <Grip Motion>

In the present disclosure, an actuation motion may be performed through the structural coupling between the operation member 540 and the second jaw 2102, instead of including a separate jaw-driving pulley.

In detail, before the end tool 2100 is driven (i.e., before stapling), the second jaw 2102 is subjected to an elastic force in a direction of maintaining an open state with respect to the first jaw 2101 by the elastic member 2109.

In this state, as the operation member 540 is moved toward the distal end 2104 by the driving of the first staple pulley 2181 and the second staple pulley 2191, the clamp 546 of the operation member 540 is drawn into the second jaw 2102 along an inclined surface forming the guide part 2102c of the second jaw 2102. In this process, when the operation member 540 is moved in the direction of an arrow A1 of FIG. 46B, the clamp 546 of the operation member 540 presses the second jaw 2102, which causes the second jaw 2102 to rotate in the direction of an arrow B1 of FIG. 46B and begin to close with respect to the first jaw 2101 while rotating.

By the driving of the first staple pulley 2181 and the second staple pulley 2191, the operation member 540 is sequentially moved in the direction of an arrow A2 of FIG. 46C and the direction of an arrow A3 of FIG. 46D, and accordingly, the clamp 546 of the operation member 540 is drawn into the second jaw 2102 and presses the second jaw 2102, which causes the second jaw 2102 to rotate in the direction of an arrow B2 of FIG. 46C and the direction of an arrow B3 of FIG. 46D, thereby completing the closing motion.

Even thereafter, by the driving of the first staple pulley 2181 and the second staple pulley 2191, the operation member 540 continues to move along the passageway 2102d of the second jaw 2102 toward the distal end of the second jaw 2102, thereby performing stapling and cutting motions.

The pulley 2111, which is a first jaw pulley, is formed in the shape of a rotatable disk as a whole, and may be formed to be rotatable around the rotation shaft 2141, which is an end tool jaw pulley rotation shaft. In addition, the pulley 2111 may be formed integrally with the first jaw 2101. For example, the pulley 2111 may be formed in a disk shape at the distal end of the first jaw 2101 and formed to move integrally with the first jaw 2101. In an optional embodiment, it is of course possible that the pulley 2111 may be provided separately from the first jaw 2101.

Meanwhile, the pulley 2121, which is a pulley for the retraction wire, may be formed in the shape of a rotatable disk as a whole and may be formed to be rotatable around the rotation shaft 2141, which is an end tool jaw pulley rotation shaft.

The coupling relationship between the components described above is as follows.

The rotation shaft 2141, which is an end tool jaw pulley rotation shaft, may be sequentially inserted through an axis of the pulley 2111, the shaft pass-through part 2191c of the second staple pulley 2191, and an axis of the pulley 2121.

The rotation shaft 2145, which is a jaw rotation shaft, may be sequentially inserted through the first jaw coupling part 2101e of the first jaw 2101 and the second jaw coupling part 2102b.

Here, the pulley 2111 and the pulley 2121 may rotate around the rotation shaft 2141, which is an end tool jaw pulley rotation shaft. The second jaw 2102 may rotate around the rotation shaft 2145, which is a jaw rotation shaft, with respect to the first jaw 2101, and as described above, the second jaw 2102 may rotate by a driving force through the operation member 540 and the elastic member 2109. The pulley 2111 and the first jaw 2101 have different shafts of rotation. Similarly, the pulley 2121 and the second jaw 2102 have different shafts of rotation.

### (Cartridge)

Hereinafter, the cartridge 500 of the surgical instrument 2000 of FIG. 2 will be described in more detail.

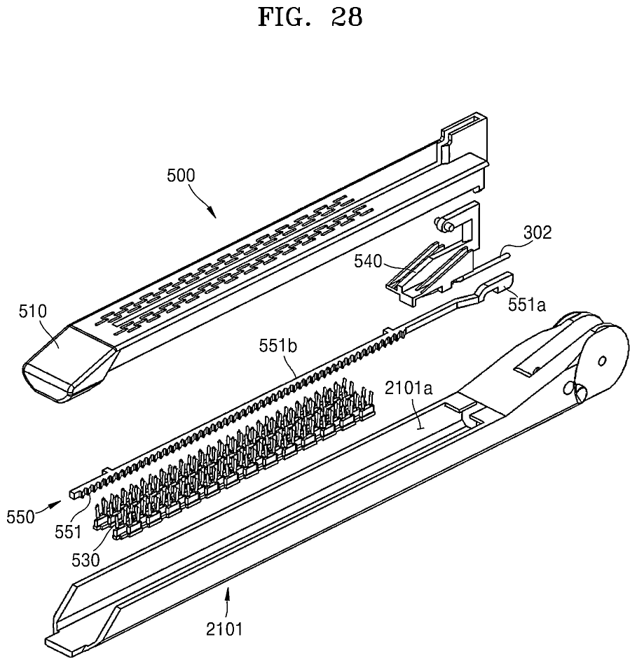

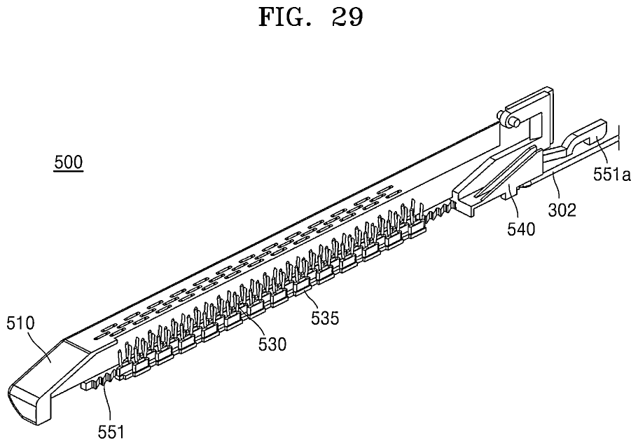

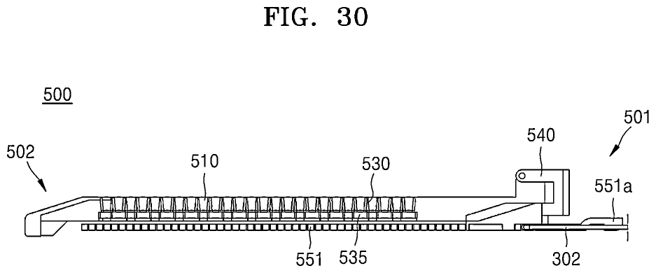

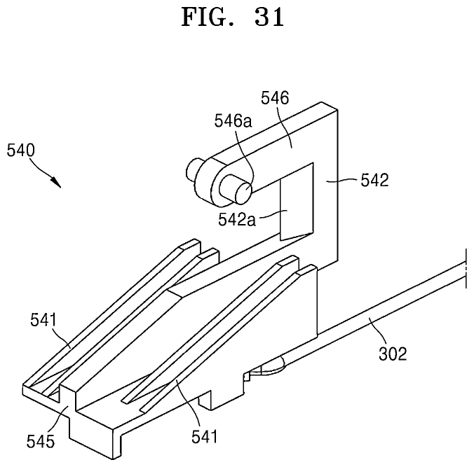

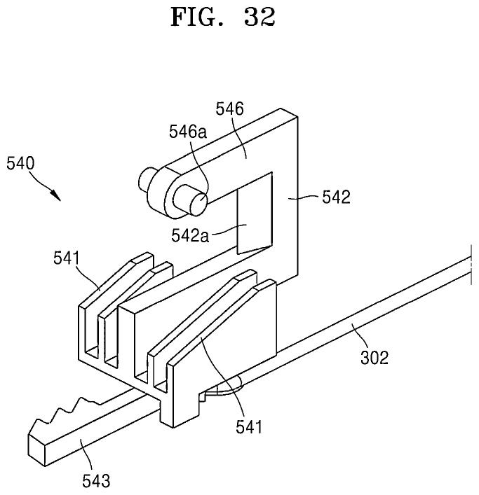

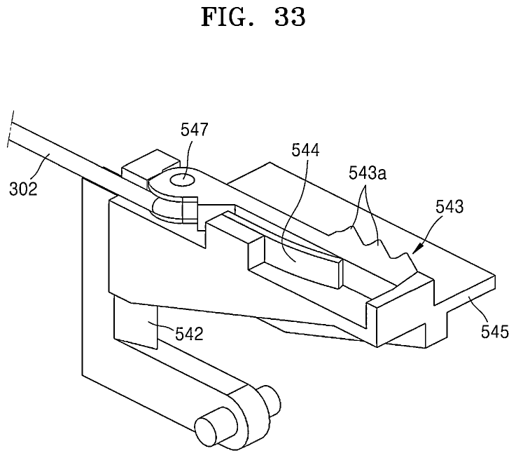

FIG. 28 is a perspective view illustrating the first jaw and the cartridge of the surgical instrument of FIG. 2. FIG. 29 is an exploded perspective view illustrating the cartridge of FIG. 28, and FIG. 30 is a side cross-sectional view illustrating the cartridge of FIG. 28. FIGS. 31 and 32 are perspective views of an operation member of the cartridge of FIG. 28 viewed from one direction. FIGS. 33 and 34 are perspective views of the operation member of the cartridge of FIG. 28 viewed from another direction.

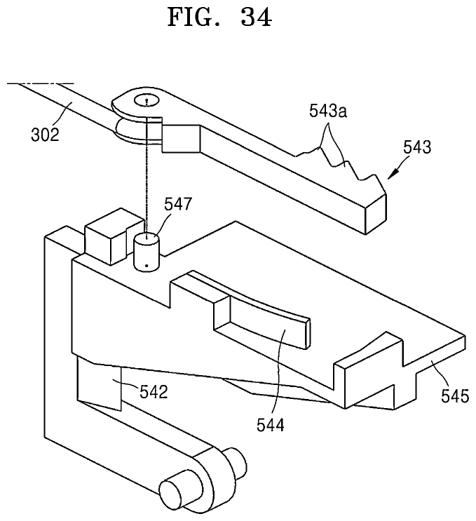

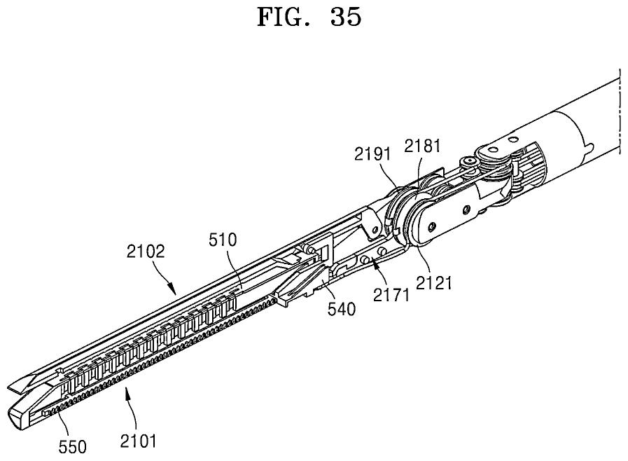

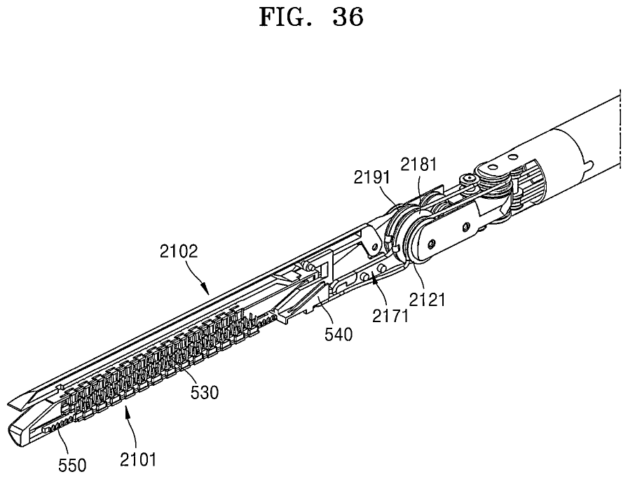

FIGS. 35 and 36 are perspective cross-sectional views illustrating a stapling-related structure of the end tool of the surgical instrument of FIG. 2.

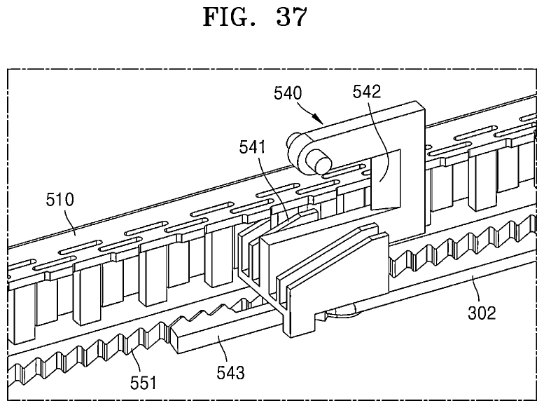

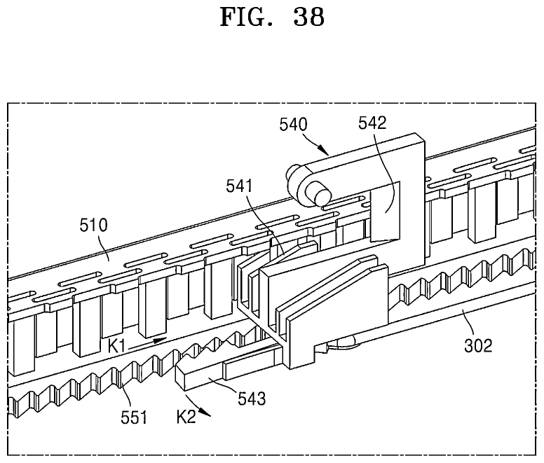

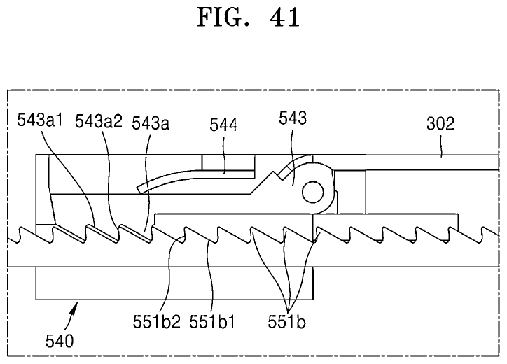

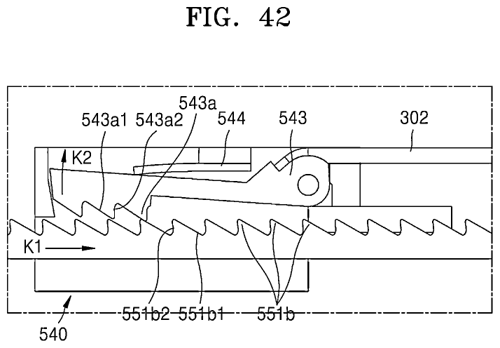

FIGS. 37 to 40 are perspective views illustrating a ratchet drive operation of the end tool of FIG. 35, and FIGS. 41 and 42 are plan views illustrating the ratchet drive operation of the end tool of FIG. 35.

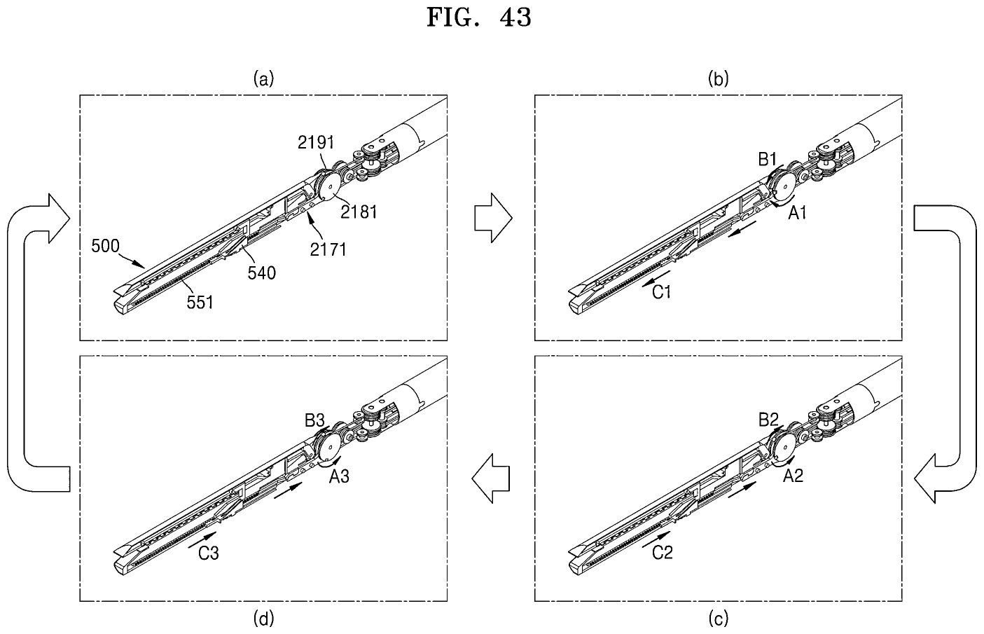

FIG. 43 is a perspective view illustrating an entire ratchet drive operation of the end tool of FIG. 35.

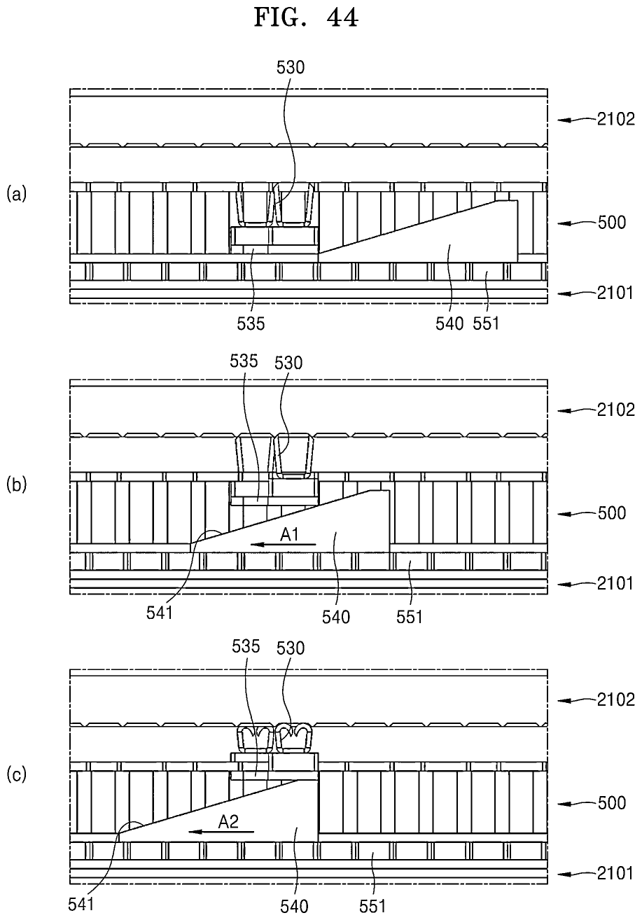

FIGS. 44 and 45 are cross-sectional views illustrating the entire stapling motion of the end tool of FIG. 35.

FIG. 46 is a view illustrating a process in which the operation member is coupled to the second jaw.

Referring to FIGS. 28 to 45 and the like, the cartridge 500 may be disposed in the first jaw 2101, and for example, the cartridge 500 may be disposed by being coupled to the cartridge accommodation part 2101b of the first jaw 2101. For example, the cartridge 500 may be integrally formed with the first jaw 2101 while the operation member 540 is connected to the retraction wire 302.

In an optional embodiment, the cartridge 500 may be formed to be mountable to and dismountable from the first jaw 2101. For example, the retraction wire 302 to be described later and the operation member 540 may be formed to be couplable to and decouplable from each other, rather than being integrally connected to each other, thereby facilitating the decoupling or coupling of the cartridge 500 from and to the first jaw 2101.

The cartridge 500 includes a plurality of staples 530 and a blade 542 therein, and performs tissue stapling and cutting. Here, the cartridge 500 may include a cover 510, the staples 530, withdrawal members 535, the operation member 540, and the reciprocating assembly 550.

The cartridge accommodation part 2101b of the first jaw 2101 may be formed to accommodate the reciprocating assembly 550, the operation member 540, and the staples 530. Here, the cartridge accommodation part 2101b may have a cross section in the shape of a concave groove with bottom and side surfaces, and may be formed in an approximately “U” shape.

The cover 510 may be formed to cover an upper portion of the cartridge accommodation part 2101b of the first jaw 2101. Staple holes through which the plurality of staples 530 may be ejected to the outside may be formed in the cover 510. As the staples 530, which are accommodated inside the cartridge accommodation part 2101b before a stapling operation, are pushed and raised upward by the operation member 540 during a stapling motion, and pass through the staple holes of the cover 510 to be withdrawn out of the cartridge 500, stapling may be performed.

Meanwhile, a slit may be formed in the cover 510 along a longitudinal direction of the cover 510. The blade 542 of the operation member 540 may protrude out of the cartridge 500 through the slit. As the blade 542 of the operation member 540 passes along the slit, staple-completed tissue may be cut.

The plurality of staples 530 may be disposed inside the cartridge accommodation part 2101b of the first jaw 2101. As the operation member 540 to be described later linearly moves in one direction, the plurality of staples 530 are sequentially pushed and raised from the inside of the cartridge accommodation part 2101b of the first jaw 2101 to the outside, thereby performing suturing, that is, stapling. Here, the staples 530 may be made of a material that may include titanium, stainless steel, or the like.

Meanwhile, the withdrawal members 535 may be further disposed between the cartridge accommodation part 2101b of the first jaw 2101 and the staples 530. In other words, it may be said that the staple 530 is disposed above the withdrawal member 535. In this case, the operation member 540 is linearly moved in one direction to push and raise the withdrawal member 535, and the withdrawal member 535 may push and raise the staple 530.

As such, the operation member 540 may be described as pushing and raising the staples 530 in both the case in which the operation member 540 directly pushes and raises the staples 530 and the case in which the operation member 540 pushes and raises the withdrawal members 535 and the withdrawal members 535 pushes and raises the staples 530 (i.e., the operation member 540 indirectly pushes and raises the staples 530).

The reciprocating assembly 550 may be disposed on the inner lower side of the cartridge accommodation part 2101b of the first jaw 2101. The reciprocating assembly 550 may include one or more reciprocating members 551. In an embodiment, it is illustrated that one reciprocating member 551 is provided, but a plurality of reciprocating members 551 may be provided.

In an embodiment, the reciprocating member 551 may be a rack. The reciprocating member 551 may include recesses 551b and the coupling part 551a. In detail, the reciprocating member 551 may be formed in the shape of an elongated bar, and a plurality of recesses 551b having a sawtooth shape may be formed on one surface thereof. The recess 551b may be formed to be in contact with the operation member 540 to be described later, in particular, the ratchet member 543 of the operation member 540. In other words, the reciprocating member 551 may include the plurality of recesses 551b shaped to engage with ratchets 543a of the ratchet member 543.

Meanwhile, although not shown in the drawings, in addition to a rack shape, the reciprocating member 551 may be provided as various shapes of members, which are directly or indirectly connected to the staple pulley assembly 2160 and may perform a linear reciprocating motion according to a rotational motion of the staple pulley assembly 2160. For example, the reciprocating member 551 may be in the form of a clutch in which recesses are not present.

Here, the reciprocating member 551 is not fixedly coupled to the other components of the cartridge 500, and may be formed to be movable relative to the other components of the cartridge 500. For example, the reciprocating member 551 may perform a reciprocating linear movement with respect to the cartridge accommodation part 2101b and the cover 510 coupled to the cartridge accommodation part 2101b.

Meanwhile, in the reciprocating member 551, the coupling part 551a may be formed at a proximal end side adjacent to the pulley 2111, and the coupling part 551a may be fastened and coupled to the staple link assembly 2170 of the end tool 2100. Thus, when the staple link assembly 2170 performs a reciprocating linear movement in the extension direction (i.e., the Y-axis direction) of the connection part 400, the reciprocating member 551 coupled thereto may also perform a reciprocating linear movement in the extension direction (i.e., the Y-axis direction) of the connection part 400. This will be described in more detail later.

The operation member 540 may be disposed inside the cartridge accommodation part 2101b of the first jaw 2101. The operation member 540 is formed to be in contact with the reciprocating member 551, and may be formed to linearly move in one direction according to the reciprocating linear movement of the reciprocating member 551. In other words, the operation member 540 interacts with the reciprocating member 551 to perform stapling and cutting motions while moving in the extension direction of the connection part 400.

The operation member 540 may include a wedge 541, the blade 542, the ratchet member 543, an operation part elastic member 544, a body 545, and the clamp 546.

The body 545 may be formed in the shape of an elongated square column, and forms a base of the operation member 540.

The wedge 541 is formed on at least one side of the body 545, and may be formed to have a certain inclined surface. That is, the wedge 541 may be formed to be inclined to a certain extent in the extension direction of the connection part 400. In other words, the wedge 541 may be formed to have a greater height at a proximal end (see 501 of FIG. 30) side of the cartridge 500 than a distal end (see 502 of FIG. 30) side of the cartridge 500. In the drawing, it is illustrated that two wedges 541 are formed on each side of the body 545 in the left and right direction, but the concept of the present disclosure is not limited thereto, and the wedge 541 may be formed in various numbers and shapes depending on the shape of the staple 530 or the withdrawal member 535 that is in contact with the wedge 541.

The wedge 541 may be formed to be in contact with the withdrawal members 535 or the plurality of staples 530 in turn and may serve to sequentially push and raise the staples 530. As shown in FIG. 45 or the like to be described later and elsewhere herein, the operation member 540 may serve to withdraw the staples 530 to the outside of the cartridge 500 by sequentially pushing and raising the staples 530 while moving toward the distal end.

The blade 542 may be formed on one side of the wedge 541, more specifically, on the proximal end 501 side of the wedge 541. An edge 542a formed to be sharp to cut tissue is formed in one region of the blade 542. As at least a portion of the edge 542a is withdrawn out of the first jaw 2101 and the cartridge 500, tissue disposed between the first jaw 2101 and the second jaw 2102 may be cut. The edge 542a of the blade 542 may be always withdrawn out of the first jaw 2101. Alternatively, the edge 542a of the blade 542 may normally be accommodated inside the first jaw 2101 or inside the cartridge 500, and may be withdrawn out of the first jaw 2101 only when the operation member 540 is moved in a longitudinal direction.

The ratchet member 543 is formed on one side of the wedge 541, more specifically, below the wedge 541, and may be formed to face the reciprocating member 551 to be described later. The ratchet member 543 may be formed in the shape of a bar and may include a plurality of ratchets 543a on one surface. The operation member 540 is moved only in one direction (i.e., toward the distal end) with respect to the reciprocating member 551 by the ratchet member 543. The ratchets 543a of the ratchet member 543 may be formed to be in contact with the recess 551b of the reciprocating member 551 described above. Meanwhile. the ratchet member 543 may be formed to rotationally move around a rotation shaft 547. For example, the ratchet member 543 may be formed to rotationally move by being inserted into the rotation shaft 547 formed in a protruding shape on the body 545 of the operation member 540. The rotation shaft 547 may be disposed closer to the proximal end 501 than the plurality of ratchets 543a.

Meanwhile, the retraction wire 302 may be connected to the ratchet member 543, and by pulling the retraction wire 302, the ratchet member 543 may rotationally move around the rotation shaft 547. As a specific example, the retraction wire 302 may be connected to one region around the rotation shaft 547. The operation member 540 may be moved backward (toward the proximal end) through the retraction wire 302.

The operation part elastic member 544 is formed on one side of the body 545 or the wedge 541 and serves to apply a certain elastic force to the ratchet member 543. As an example, the operation part elastic member 544 may be formed such that one region is in contact with the body 545, and another region that is different from the one region is in contact with the ratchet member 543. The operation part elastic member 544 may apply an elastic force in a direction in which the ratchet member 543 comes into close contact with the reciprocating member 551. To this end, the operation part elastic member 544 may be formed in the form of a leaf spring, and may be provided in various forms capable of providing a certain elastic force to the ratchet member 543, such as a coil spring, a dish spring, and the like. In addition, although not shown in the drawings, in another optional embodiment, the operation part elastic member 544 may be integrally formed with the operation member 540.

The clamp 546 may be formed on one side of the blade 542 and may be formed in a shape that is approximately parallel to the body 545 or the wedge 541. In addition, a protrusion 546a may be formed at one end portion of the clamp 546, and the protrusion 546a may be moved along the passageway 2102d of the second jaw 2102.

Here, as an example, the clamp 546 may be formed parallel to the wedge 541 and configured to have an overall ‘U’ shape, thereby better withstanding a strong pressure applied to the wedge 541 during the stapling motion.

In addition, similarly as described above, when the operation member 540 moves forward, the clamp 546 is inserted into the interior of the second jaw 2102 to bring the second jaw 2102 closer to the first jaw 2101 and cause the second jaw 2102 and the first jaw 2101 to naturally close.

Here, the ratchet 543a of the ratchet member 543 may be formed such that a first surface 543a1 (specifically, the surface at the distal end side) is formed to have a gentle slope with a certain angle, and a second surface 543a2 (specifically, the surface at the proximal end side) is formed to be vertical or near vertical.

In addition, in order to engage with the ratchet 543a of the ratchet member 543, the recess 551b of the reciprocating member 551 may also be formed such that a first surface 551b1 (specifically, the surface at the proximal end 501 side) is formed to have a gentle slope with a certain angle, and a second surface 551b2 (specifically, the surface at the distal end 502 side) is formed to be vertical or near vertical.

In a state in which the reciprocating member 551 and the ratchet member 543 are coupled to each other (or engaged or in close contact with each other), the inclined first surface 543a1 of the ratchet 543a and the inclined first surface 551b1 of the recess 551b may be formed to face each other (that is, in contact with each other). In addition, the vertically formed second surface 543a2 of the ratchet 543a and the vertically formed second surface 551b2 of the recess 551b may be disposed to face each other (i.e., in contact with each other).

With this configuration, in a state in which the ratchet 543a and the recess 551b are coupled to (or engaged with) each other, the ratchet 543a and the recess 551b may be allowed to move only in one direction, acting as a kind of ratchet.

As an example, when it is assumed that the reciprocating member 551 is in a fixed state, the operation member 540 is movable in a direction in which the second surface 543a2 and the second surface 551b2, which are vertically formed, are away from each other, but when the second surface 543a2 and the second surface 551b2 are in contact with each other, the operation member 540 is not movable in a direction in which the second surface 543a2 and the second surface 551b2 are closer to each other.

In other words, when the reciprocating member 551 is moved toward the distal end 502 in a state in which the reciprocating member 551 and the ratchet member 543 are coupled to each other (or engaged or in close contact with each other), the ratchet member 543 is moved together toward the distal end 502 by the reciprocating member 551. That is, the vertically formed second surface 551b2 of the reciprocating member 551 pushes the vertically formed second surface 543a2 of the operation member 540 such that the ratchet member 543 is moved together toward the distal end 502 by the reciprocating member 551.

In contrast, when the reciprocating member 551 is moved toward the proximal end 501 in a state in which the reciprocating member 551 and the ratchet member 543 are coupled to each other (or engaged or in close contact with each other), only the reciprocating member 551 is moved alone toward the proximal end 501 while the ratchet member 543 is fixed. That is, the inclined first surface 551b1 of the reciprocating member 551 is moved along the inclined first surface 543a1 of the operation member 540 in a state in which the operation member 540 is fixed, so that only the reciprocating member 551 is moved alone toward the proximal end 501.

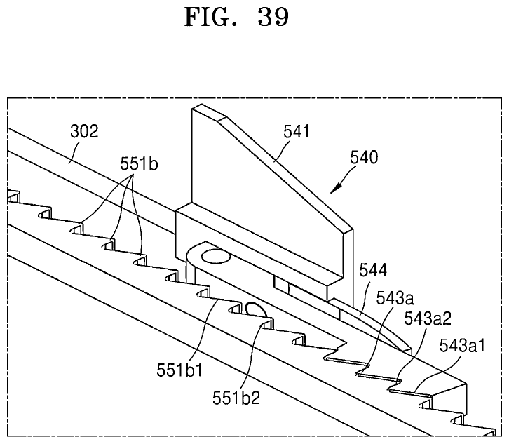

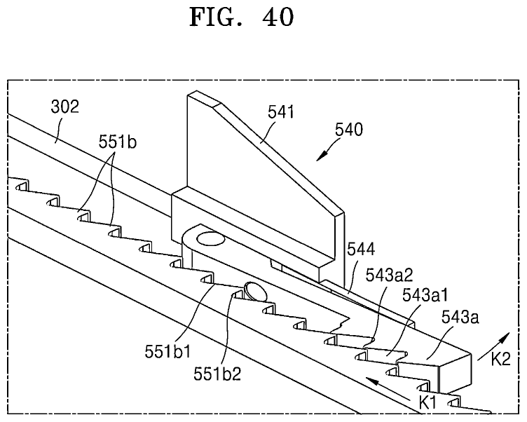

Referring to FIGS. 39 to 42, when the reciprocating member 551 is moved toward (in the direction of an arrow K1 of FIGS. 40 and 42) of the proximal end 501 in the state of FIGS. 39 and 41, as the inclined first surface 551b1 of the reciprocating member 551 is moved along the inclined first surface 543a1 of the operation member 540, the ratchet member 543 is pushed as a whole in the direction of an arrow K2 of FIG. 40. In addition, at this time, the operation part elastic member 544 is elastically deformed to a certain extent.

In this state, when the reciprocating member 551 is further moved toward the proximal end 501, and the inclined first surface 551b1 of the reciprocating member 551 is moved beyond an end of the inclined first surface 543a1 of the operation member 540, the recess 551b of the reciprocating member 551 meets the next ratchet 543a of the ratchet member 543. In this case, since the operation part elastic member 544 applies an elastic force in a direction in which the ratchet member 543 comes into close contact with the reciprocating member 551, front surfaces of the reciprocating member 551 and the ratchet member 543 are brought into close contact with each other again.

As a result, the cartridge 500 is accommodated in the cartridge accommodation part 2101b of the first jaw 2101, and in this case, the reciprocating member 551 of the cartridge 500 is coupled to the staple link assembly 2170 of the end tool 2100. Accordingly, the rotational motion of the staple pulley assembly 2160 of the end tool 2100 is converted into a linear motion of the reciprocating member 551 through the staple link assembly 2170.

In this case, when the coupling part 551a of the reciprocating member 551 is connected to the staple pulley assembly 2160 through the staple link assembly 2170, and the staple pulley assembly 2160 is rotated alternately in the clockwise/counterclockwise directions, the reciprocating member 551 may be repeatedly moved forward and backward. In addition, when the reciprocating member 551 is moved forward, the operation member 540 may be moved forward together with the reciprocating member 551, and when the reciprocating member 551 is moved backward, only the reciprocating member 551 may be moved backward and the operation member 540 may remain stationary in place. As the operation member 540 moves forward while repeating this process, the staple 530 may be stapled by the wedge 541 while the blade 542 cuts the stapled tissue.

This will be described in more detail as follows.

### (Stapling and Cutting Motions)

Referring to FIG. 43, a method of driving the surgical instrument according to an embodiment of the present disclosure is described as follows.

First, when the first staple pulley 2181 is rotated in the clockwise direction and the second staple pulley 2191 is rotated in the counterclockwise direction, the staple link assembly 2170 connected to the staple pulley assembly 2160 and the reciprocating assembly 550 of the cartridge 500 connected to the staple link assembly 2170 are moved toward the distal end 502 of the cartridge 500.

In addition, when the reciprocating assembly 550 is moved toward the distal end 502 of the cartridge 500, the operation member 540 in contact with the reciprocating assembly 550 is moved toward the distal end 502 of the cartridge 500 together with the reciprocating assembly 550.

In addition, as the operation member 540 is moved toward the distal end 502 of the cartridge 500, the blade 542 of the operation member 540 is moved toward the distal end 502 of the cartridge 500 while the operation member 540 ejects the staples 530 out of the cartridge 500.

Meanwhile, when the first staple pulley 2181 is rotated in the counterclockwise direction and the second staple pulley 2191 is rotated in the clockwise direction, the reciprocating assembly 550 of the staple link assembly 2170 connected to the staple pulley assembly 2160 and the cartridge 500 connected to the staple link assembly 2170 are moved toward the proximal end 501 of the cartridge 500, and at this time, the operation member 540 remains stationary.

In addition, as the above operations are repeatedly performed, a stapling motion by the wedge 541 and a cutting motion by the blade 542 are simultaneously performed.

This will be described in more detail as follows.

First, in the state as shown in FIG. 43A, as shown in FIG. 43B, when the first staple pulley 2181 is rotated in the direction of an arrow A1 (i.e., in the clockwise direction) and the second staple pulley 2191 is rotated in the direction of an arrow B1 (i.e., in the counterclockwise direction), the staple link assembly 2170 connected to the first and second staple pulleys 2181 and 2191 and the reciprocating member 551 coupled to the staple link assembly 2170 are moved in the direction of an arrow C1 (i.e., toward the distal end) In this state, since the reciprocating member 551 and the operation member 540 are in close contact with each other by the elastic member (see 544 of FIG. 42), when the reciprocating member 551 is moved in the direction of the arrow C1, the operation member 540 is also moved in the direction of the arrow C1 together with the reciprocating member 551.

On the other hand, as shown in FIG. 43C, when the first staple pulley 2181 is rotated in the direction of an arrow A2 (i.e., in the counterclockwise direction) and the second staple pulley 2191 is rotated in the direction of an arrow B2 (i.e., in the clockwise direction), the staple link assembly 2170 connected to the first and second staple pulleys 2181 and 2191 and the reciprocating member 551 coupled to the staple link assembly 2170 are moved in the direction of an arrow C2 (i.e., toward the proximal end). In this state, due to the coupling structure of the ratchet member 543 and the reciprocating member 551, even when the reciprocating member 551 is moved in the C2 direction, only the ratchet member 543 is repeatedly spaced apart from and in contact with the reciprocating member 551 to a certain extent as the operation part elastic member 544 is repeatedly elastically deformed and restored in a state in which the overall position of the operation member 540 remains unchanged. That is, (referring to FIGS. 40 and 42), even when the reciprocating member 551 is moved in the direction of the arrow C2, the operation member 540 remains stationary in place when viewed from the X-axis direction.

Meanwhile, as shown in FIG. 43D, when the first staple pulley 2181 is further rotated in the direction of an arrow A3, and the second staple pulley 2191 is further rotated in the direction of an arrow B3, only the staple link assembly 2170 and the reciprocating member 551 connected thereto are further moved in the direction of an arrow C3.

In this state, when the first staple pulley 2181 stops rotating, the staple link assembly 2170, the reciprocating member 551, and the operation member 540 also stop moving.

When the first staple pulley 2181 and the second staple pulley 2191 are alternately rotated in the clockwise and counterclockwise directions while repeating the above process, the reciprocating member 551 is repeatedly moved forward and backward, and the operation member 540 repeats moving forward and stopping, and as a result, the operation member 540 is moved toward the distal end 502. In addition, as the operation member 540 is moved toward the distal end 502, a stapling motion by the wedge 541 and a cutting motion by the blade 542 are simultaneously performed.

Hereinafter, a stapling motion of the surgical instrument according to an embodiment of the present disclosure will be described.

FIG. 44 is a cross-sectional view illustrating a stapling motion of the end tool of FIG. 35 for each section, and FIG. 45 is a cross-sectional view illustrating an entire stapling motion of the end tool of FIG. 35.

Referring to FIGS. 44 and 45, in the state as shown in FIG. 44A, as the operation member 540 is moved in the direction of an arrow A1 of FIG. 44B, the wedge 541 of the operation member 540 pushes and raises the withdrawal member 535, and the withdrawal member 535 pushes and raises one side of a lower portion of the staple 530. In addition, due thereto, the staple 530 is ejected to the outside of the first jaw 2101 and the cartridge 500.

In this state, when the operation member 540 further moves in the direction of an arrow A2 of FIG. 44C, the ejected staple 530 is continuously pushed and raised by the operation member 540 while in contact with the lower surface of the second jaw 2102, such as the anvil, so that stapling is performed while both end portions of the staple 530 are bent.

As such motions are continuously performed, stapling is sequentially performed from the staple 530 on the proximal end 501 side to the staple 530 on the distal end 502 side among the plurality of staples 530, as illustrated of FIG. 45.

### <Backward Movement>

Meanwhile, in the present disclosure, the retraction wire 302 is provided to enable the backward movement of the operation member 540, i.e., the movement of the operation member 540 toward the proximal end 2105.

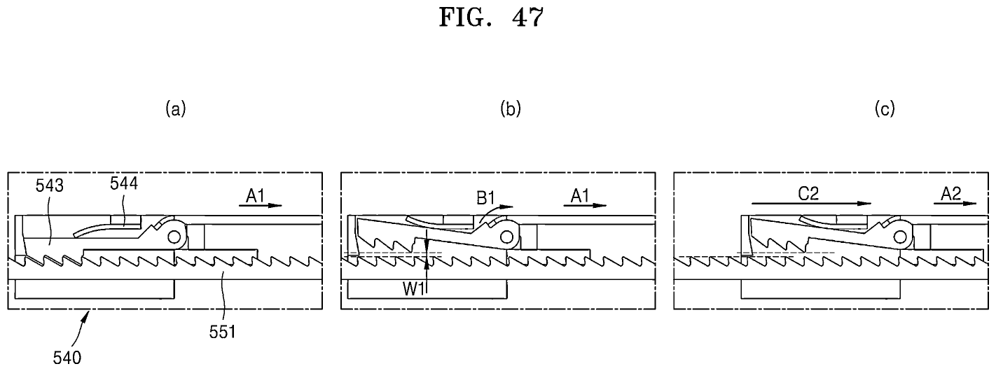

FIG. 47 is a plan view illustrating a backward movement of the operation member of the end tool of FIG. 35.

As described above, the ratchet member 543 may be formed to be rotatable around the rotation shaft 547, and as a specific example, the ratchet member 543 may be inserted into the rotation shaft 547 formed in a shape protruding from the body 545 of the operation member 540 and may rotationally move around the rotation shaft 547.

The retraction wire 302 may be connected to the ratchet member 543, and by pulling the retraction wire 302, the ratchet member 543 may rotationally move around the rotation shaft 547. As a specific example, the retraction wire 302 may be connected to one region around the rotation shaft 547.

When the retraction wire 302 is pulled in the direction of an arrow A1 of FIG. 47B, the ratchet member 543 is rotated around the rotation shaft 547 in the direction of an arrow B1 of FIG. 47B.

Here, when a certain amount or more of force is applied to the retraction wire 302, the ratchet member 543 is spaced away from the reciprocating member 551 to generate a separation gap W1, and the coupling (engagement) between the ratchet member 543 and the reciprocating member 551 is released, so that the operation member 540 becomes movable with respect to the reciprocating member 551.

In this state, when the retraction wire 302 is further pulled in the direction of an arrow A2 of FIG. 47C, the operation member 540 as a whole is moved in the direction of an arrow C2 of FIG. 47C in a state in which the ratchet member 543 is spaced away from the reciprocating member 551.

As a result, the operation member 540 can be moved backward, i.e., cab be moved toward the proximal end 2105 by the retraction wire 302.

### <Correlation Between Backward Movement and Other Motions>

Hereinafter, the correlation between the backward movement and other motions (pitch, motion, and stapling motions).

First, during a pitch motion of the end tool 2100, the retraction wire 302 should be wound or unwound accordingly. In detail, for the pitch motion, the wire 307/wire 308, which are first staple wires, should be pulled in the same direction, while the wire 309/wire 310, which are second staple wires, should be released in the same direction (or, the wire 307/wire 308, which are first staple wires, should be released in the same direction, and the wire 309/wire 310, which are second staple wires, should be pulled in the same direction). In addition, at this time, the retraction wire 302 should also be released in the same direction as the wire 307/wire 308, which are first staple wires, by the manipulation part 200 (or should be pulled). This ensures that unintended movement of the retraction wire 302 is prevented.

Next, during a yaw motion of the end tool 2100, the retraction wire 302 should be wound or unwound accordingly. In detail, for the yaw motion, one of the wire 301/wire 305, which are jaw wires should be wound, and another one thereof should be unwound. In addition, at this time, the retraction wire 302 should also be wound or unwound in response to the wire 301/wire 305, which are jaw wires by the manipulation part 200. This ensures that unintended movement of the retraction wire 302 is prevented.

Next, during a stapling motion of the end tool 2100, the retraction wire 302 should be wound or unwound accordingly. In detail, for the stapling motion, the first staple pulley 2181 should rotate in one direction, and to this end, one of the wire 307/wire 308, which are first staple wires, should be wound, and another one thereof should be unwound. In addition, the second staple pulley 2191 should be rotated in the opposite direction to the first staple pulley 2181, and to this end, one of wire 309/wire 310, which are second staple wires, should be wound, and another one thereof should be unwound. In addition, at this time, the retraction wire 302 should also be released in the same direction as the wire 307/wire 308, which are first staple wires, by the manipulation part 200 (or should be pulled). This ensures that unintended movement of the retraction wire 302 is prevented.

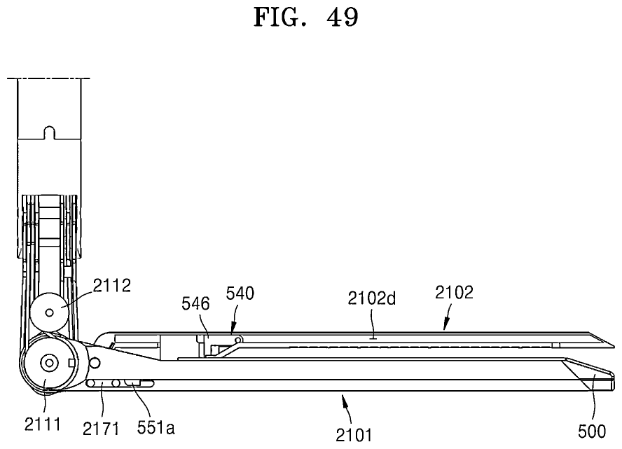

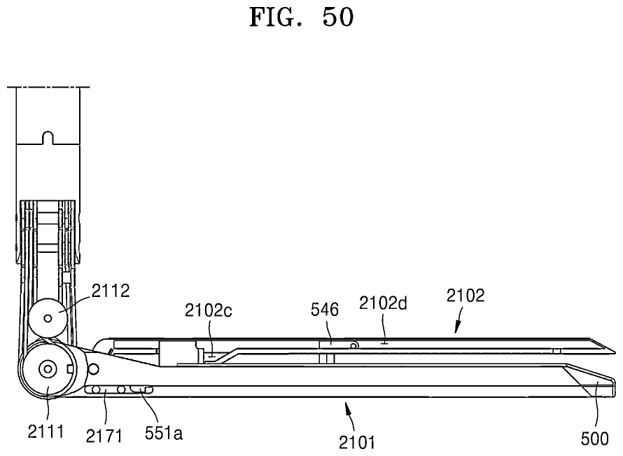

FIGS. 48 to 51 are plan views illustrating an actuation motion of the end tool of the surgical instrument of FIG. 2, and are views illustrating a process of performing an actuation motion in a state in which the jaws are yaw-rotated by +90°.

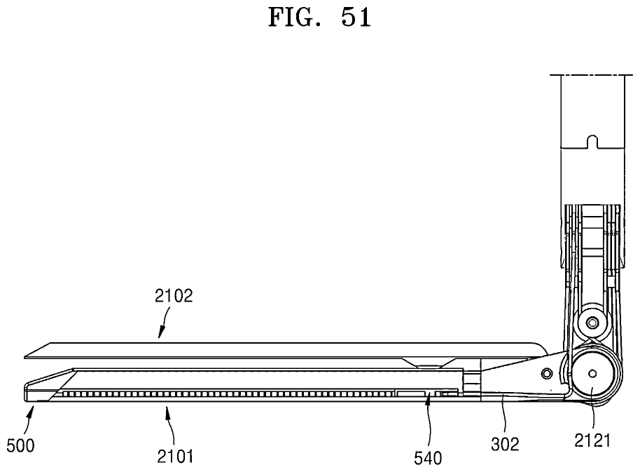

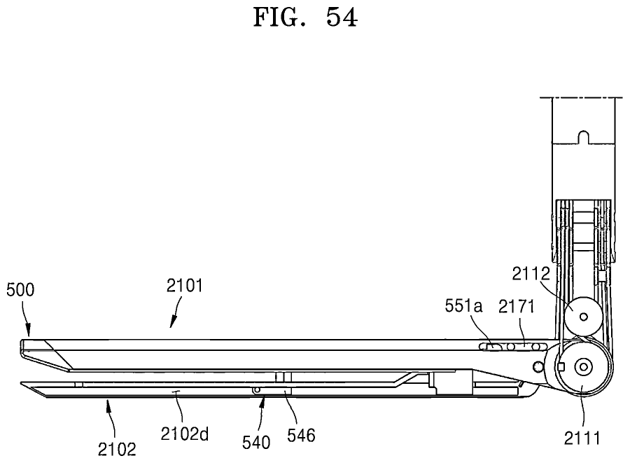

FIGS. 52 to 55 are plan views illustrating an actuation motion of the end tool of the surgical instrument of FIG. 2, and are views illustrating a process of performing an actuation motion in a state in which the jaws are yaw-rotated by −90°.

FIGS. 56 and 57 are plan views illustrating a stapling motion of the end tool of the surgical instrument of FIG. 2, and is a view illustrating a process of performing a stapling motion in a state in which the jaws are yaw-rotated by +90°.

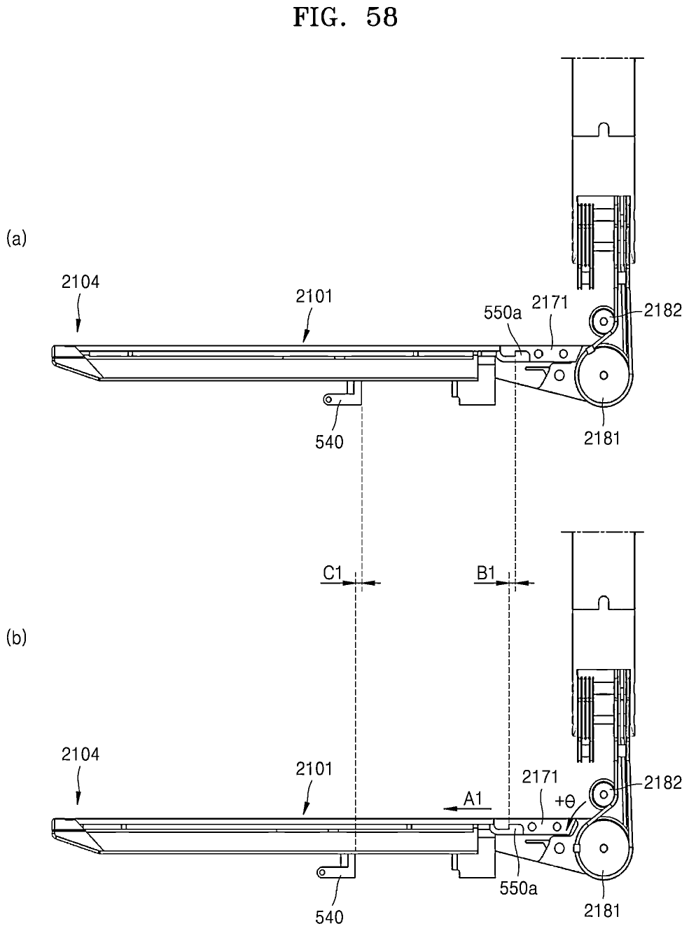

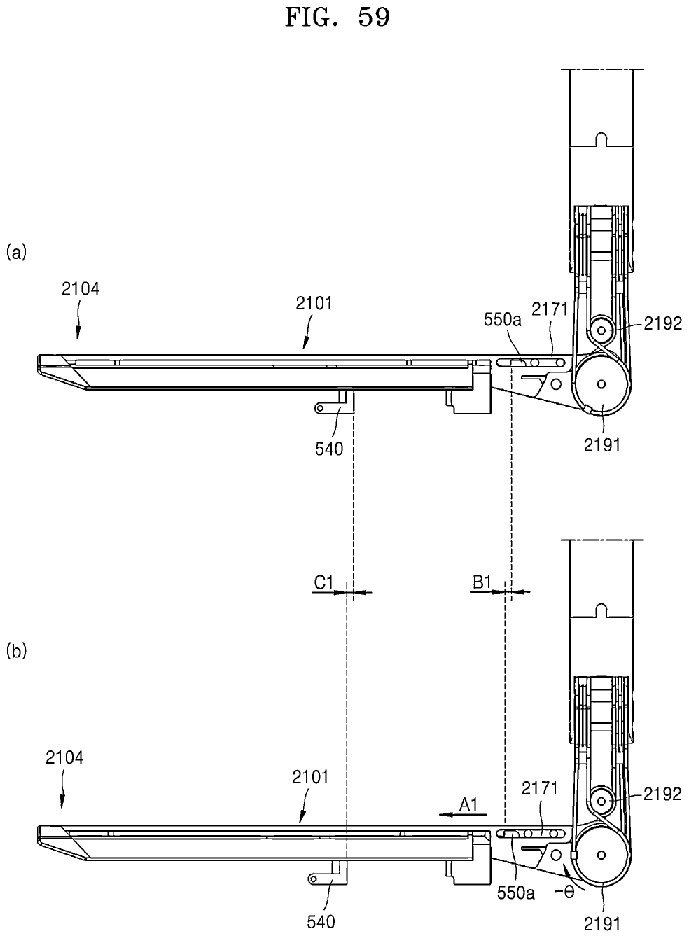

FIGS. 58 and 59 are plan views illustrating a stapling motion of the end tool of the surgical instrument of FIG. 2, and is a view illustrating a process of performing a stapling motion in a state in which the jaws are yaw-rotated by −90°.

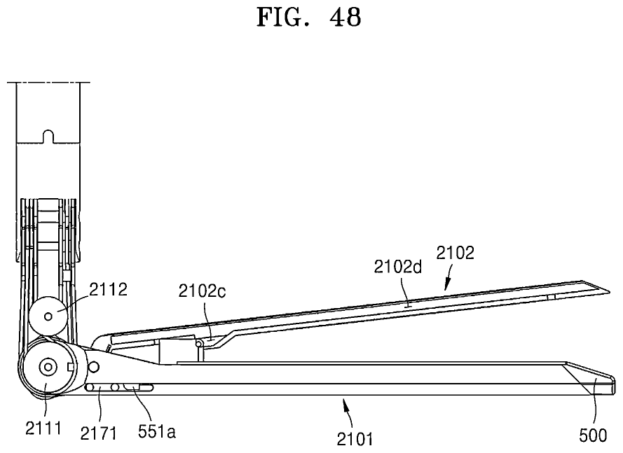

FIG. 48 is a view illustrating a state in which the jaws are yaw-rotated by +90°, and FIGS. 49 to 51 are views illustrating a process of performing an actuation motion in a state in which the jaws are yaw-rotated by +90°. In addition, FIG. 51 is a view illustrating a path of the retraction wire 302.

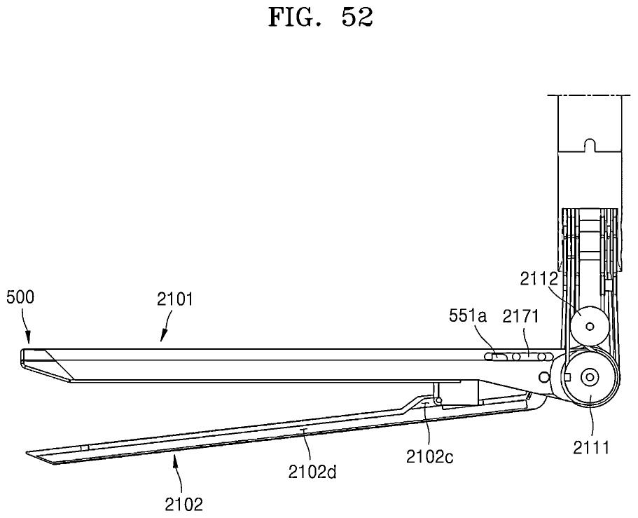

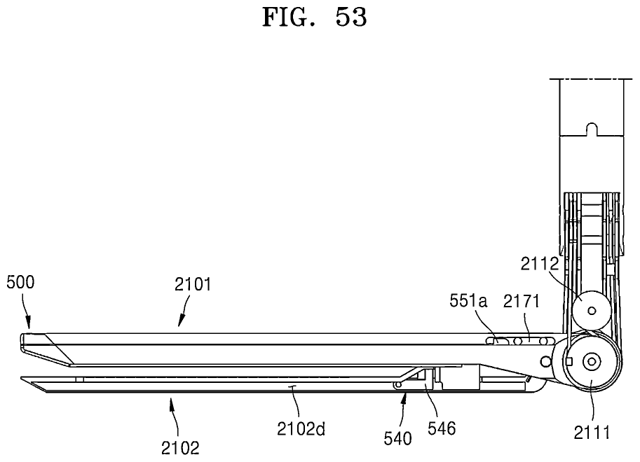

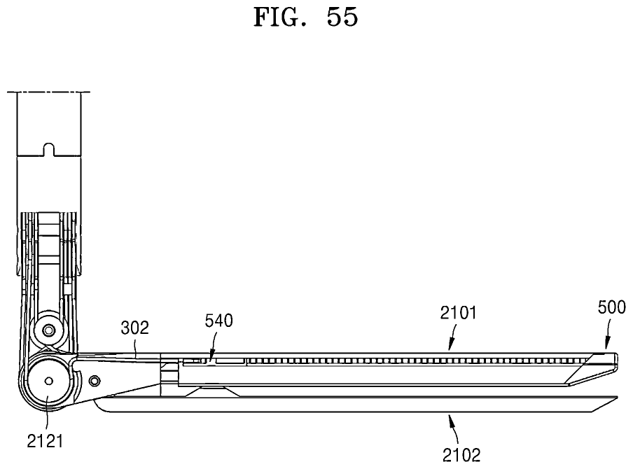

FIG. 52 is a view illustrating a state in which the jaws are yaw-rotated by −90°, and FIGS. 53 to 55 are views illustrating a process of performing an actuation motion in a state in which the jaws are yaw-rotated by −90°. In addition, FIG. 55 is a view illustrating the path of the retraction wire 302.

As shown in FIGS. 48 to 55, the end tool of the surgical instrument according to the first embodiment of the present disclosure is formed to normally perform an actuation motion even when the jaws are yaw-rotated by +90° or −90°.

FIGS. 56 and 57 are plan views illustrating stapling and cutting motions of the end tool of the surgical instrument of FIG. 2, and is a view illustrating a process of performing the stapling and cutting motions in a state in which the jaws are yaw-rotated by +90°. As shown in FIG. 56, the end tool of the surgical instrument according to the first embodiment of the present disclosure is formed to normally perform the stapling and cutting motions even when the jaws are yaw-rotated by +90°.

In detail, when the first staple pulley 2181 is rotated in an A1 direction of FIG. 56, which is a counterclockwise direction, in a state in which the first jaw 2101 is rotated by +90°, the coupling part 551a of the reciprocating member 551 connected to the first staple pulley 2181 is moved forward toward the distal end 2104, and accordingly, the operation member 540 is also moved forward toward the distal end 2104. In addition, when the second staple pulley 2191 is rotated in an A1 direction of FIG. 57, which is a clockwise direction, in a state in which the first jaw 2101 is rotated by +90°, the coupling part 551a of the reciprocating member 551 connected to the second staple pulley 2191 is moved forward toward the distal end 2104, and accordingly, the operation member 540 is also moved forward toward the distal end 2104.

As described above, the second jaw 2102 of the end tool of the surgical instrument of the present embodiment may be coupled to the first jaw 2101 and perform pitch and yaw motions together with the first jaw 2101. In addition, the second jaw 2102 may be maintained in an open state with respect to the first jaw 2101 through the elastic member 2019, and through the forward movement of the operation member 540, the operation member 540 is coupled to the second jaw 2102, so that the second jaw 2102 may be moved in a direction closer to the first jaw 2101, and the second jaw 2102 may be maintained in a closed state with respect to the first jaw 2101. In addition, after the operation member 540 is moved forward toward the distal end, if necessary, the retraction wire 302 may be pulled to move the operation member 540 backward toward the proximal end.

FIGS. 58 and 59 are plan views illustrating stapling and cutting motions of the end tool of the surgical instrument of FIG. 2, and is a view illustrating a process of performing the stapling and cutting motions in a state in which the jaws are yaw-rotated by −90°. As shown in FIG. 58, the end tool of the surgical instrument according to the first embodiment of the present disclosure is formed to normally perform the stapling and cutting motions even when the jaws are yaw-rotated by −90°.

In detail, when the first staple pulley 2181 is rotated in an A1 direction of FIG. 58, which is a counterclockwise direction, in a state in which the first jaw 2101 is rotated by −90°, the coupling part 551a of the reciprocating member 551 connected to the first staple pulley 2181 is moved forward toward the distal end 2104, and accordingly, the operation member 540 is also moved forward toward the distal end 2104. In addition, when the second staple pulley 2191 is rotated in an A1 direction of FIG. 59, which is a clockwise direction, in a state in which the first jaw 2101 is rotated by −90°, the coupling part 551a of the reciprocating member 551 connected to the second staple pulley 2191 is moved forward toward the distal end 2104, and accordingly, the operation member 540 is also moved forward toward the distal end 2104.

The content regarding the closing of the second jaw 2102 through the forward movement of the operation member 540 and the retraction wire 302, in a state in which the first jaw 2101 is rotated by −90°, is essentially the same as the description provided above for the state in which the first jaw 2101 is rotated by +90°.

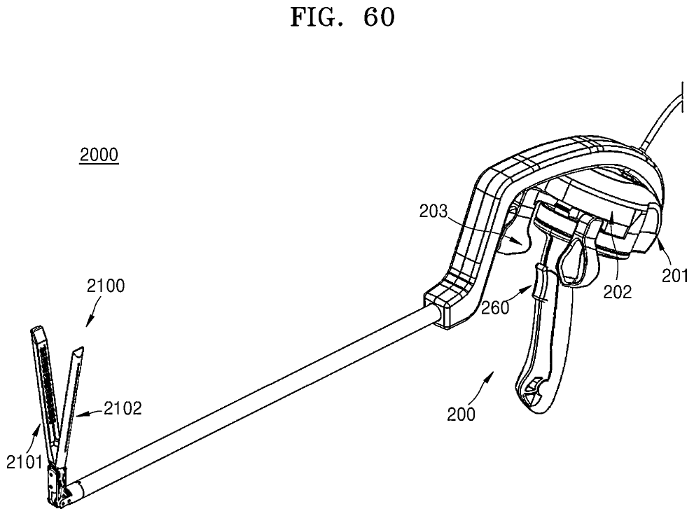

FIGS. 60 to 63 are perspective views illustrating a pitch motion of the surgical instrument of FIG. 2.

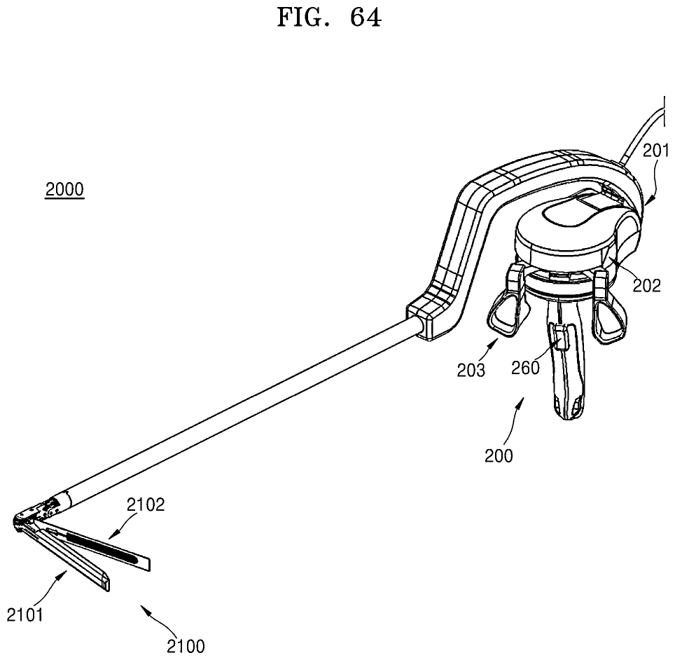

FIGS. 64 to 67 are perspective views illustrating a yaw motion of the surgical instrument of FIG. 2.

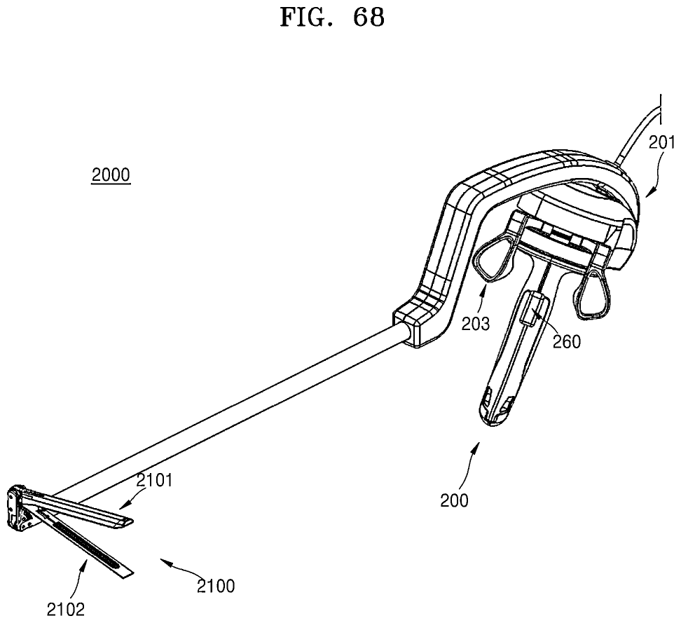

FIGS. 68 to 71 are perspective views illustrating a state in which the end tool of the surgical instrument of FIG. 2 is pitch-rotated and yaw-rotated.

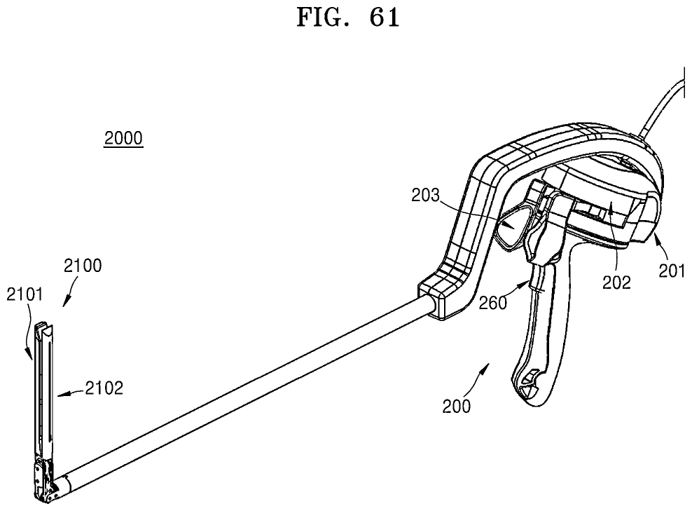

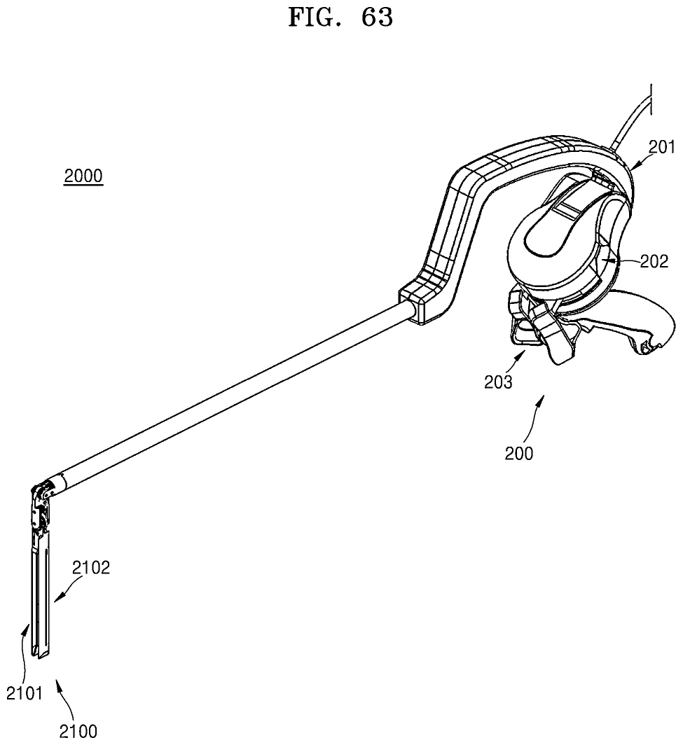

FIG. 60 is a view illustrating a state in which the jaws are pitch-rotated by −90°, and FIG. 61 is a view illustrating a process of performing an actuation motion in a state in which the jaws are pitch-rotated by −90°. FIG. 62 is a view illustrating a state in which the jaws are pitch-rotated by +90°, and FIG. 63 is a view illustrating a process of performing an actuation motion in a state in which the jaws are pitch-rotated by +90°.

Referring to FIGS. 60 to 63, it can be seen that, in performing a pitch motion, the motions of the manipulation part 200 and the end tool 2100 are intuitively matched. That is, when the manipulation part 200 is rotated in a positive (+) direction with respect to the pitch rotation axis (Y-axis), the end tool 2100 is also rotated in the positive (+) direction with respect to the pitch rotation axis (Y-axis). In addition, when the manipulation part 200 is rotated in a negative (−) direction with respect to the pitch rotation axis (Y-axis), the end tool 2100 is also rotated in the negative (−) direction with respect to the pitch rotation axis (Y-axis). Here, the rotation angle of the manipulation part 200 and the rotation angle of the end tool 2100 may be variously set according to the ratio of the pulleys.

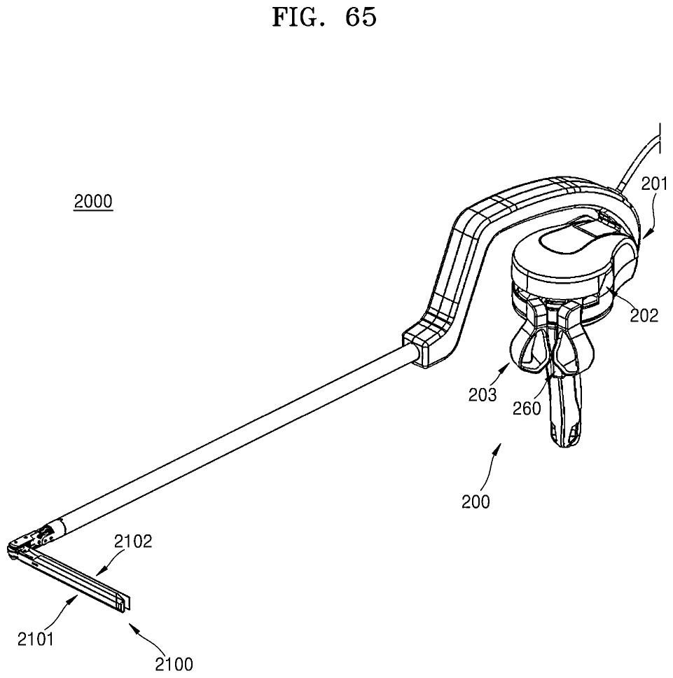

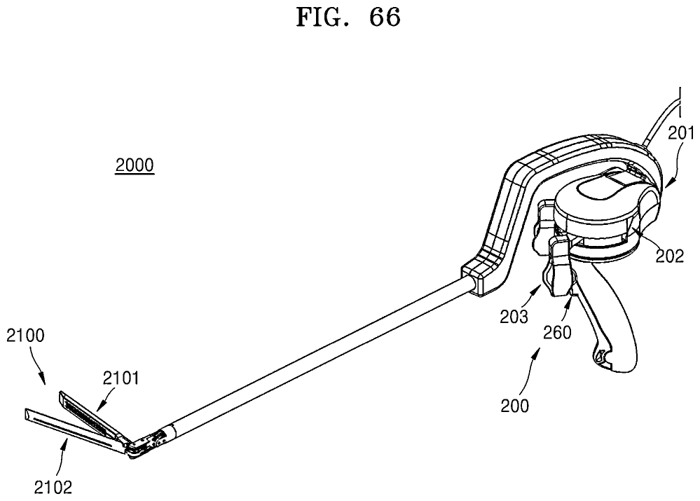

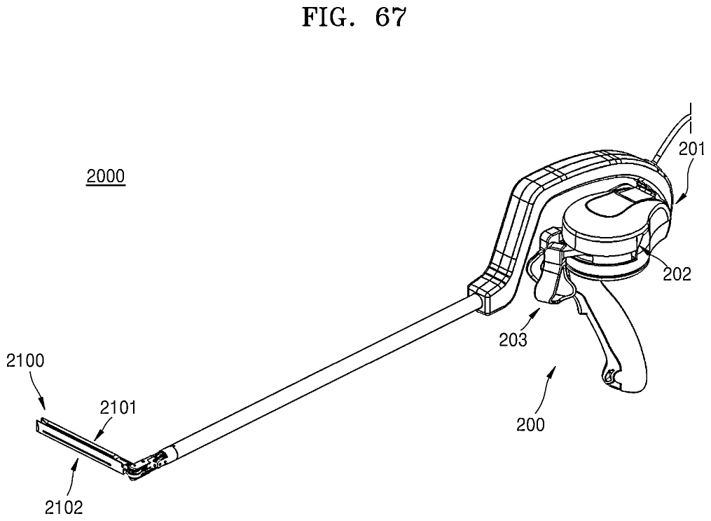

FIG. 64 is a view illustrating a state in which the jaws are yaw-rotated by +90°, and FIG. 65 is a view illustrating a process of performing an actuation motion in a state in which the jaws are yaw-rotated by +90°. FIG. 66 is a view illustrating a state in which the jaws are yaw-rotated by −90°, and FIG. 67 is a view illustrating a process of performing an actuation motion in a state in which the jaws are yaw-rotated by −90°.

Referring to FIGS. 64 to 67, it can be seen that, in performing a yaw motion, the motions of the manipulation part 200 and the end tool 2100 are intuitively matched. That is, when the manipulation part 200 is rotated in a positive (+) direction with respect to the yaw rotation axis (Z-axis), the end tool 2100 is also rotated in the positive (+) direction with respect to the yaw rotation axis (Z-axis). In addition, when the manipulation part 200 is rotated in a negative (−) direction with respect to the yaw rotation axis (Z-axis), the end tool 2100 is also rotated in the negative (−) direction with respect to the yaw rotation axis (Z-axis). Here, the rotation angle of the manipulation part 200 and the rotation angle of the end tool 2100 may be variously set according to the ratio of the pulleys.

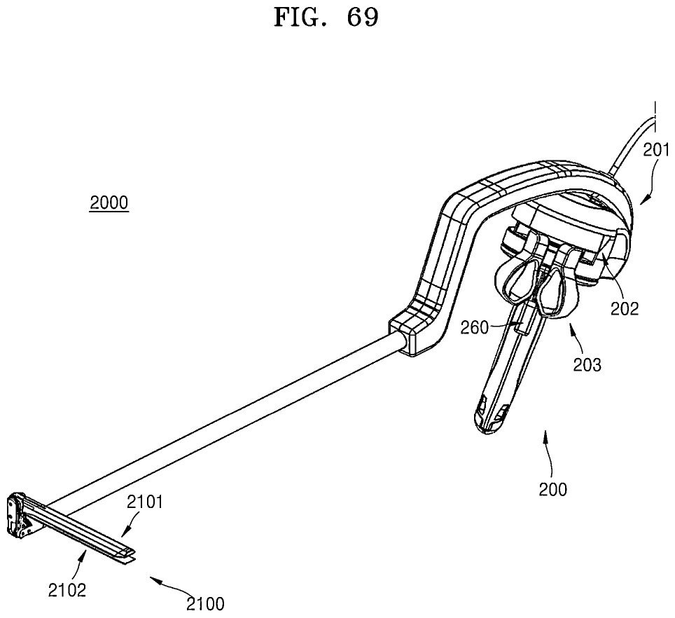

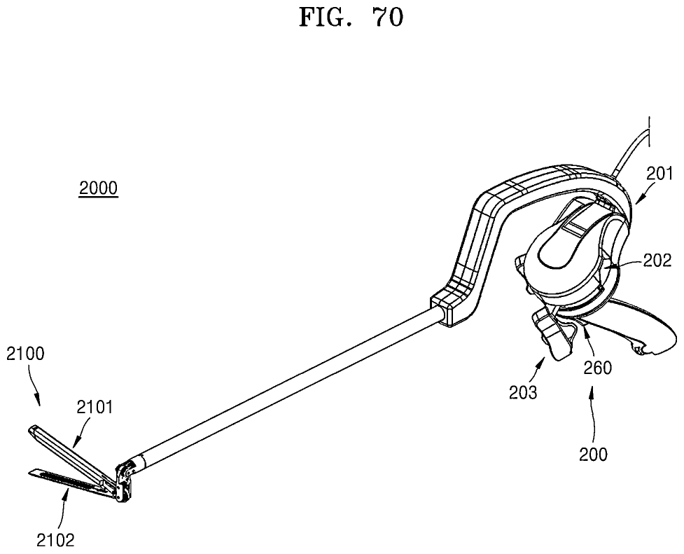

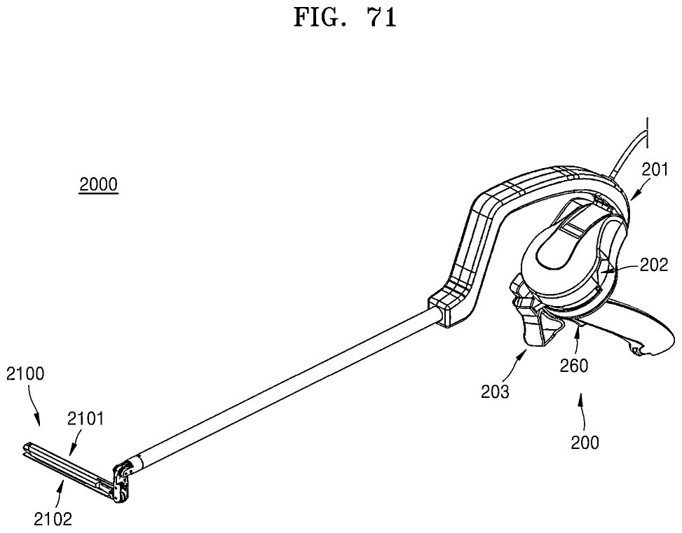

FIG. 68 is a view illustrating a state in which the jaws are pitch-rotated by −90° and at the same time yaw-rotated by +90°, and FIG. 69 is a view illustrating a process of performing an actuation motion in the state in which the jaws are pitch-rotated by −90° and at the same time yaw-rotated by +90°, FIG. 70 is a view illustrating a state in which the jaws are pitch-rotated by +90° and at the same time yaw-rotated by −90°, and FIG. 71 is a view illustrating a process of performing an actuation motion in the state in which the jaws are pitch-rotated by +90° and at the same time yaw-rotated by −90°,

Referring to FIGS. 68 to 71, it can be seen that the motions of the manipulation part 200 and the end tool 2100 are intuitively matched, even when performing the pitch and yaw motions simultaneously.

### Second Embodiment of Surgical Instrument

Hereinafter, an end tool 3100 of a surgical instrument according to a second embodiment of the present disclosure will be described. Here, the end tool 3100 of the surgical instrument according to the second embodiment of the present disclosure is different from the end tool (see 2100 of FIG. 2 or the like) of the surgical instrument according to the first embodiment of the present disclosure described above in that a configuration of a cartridge 1500 including an operation member 1540 is changed. Hereinafter, the configuration that is different from that of the first embodiment will be described in detail.

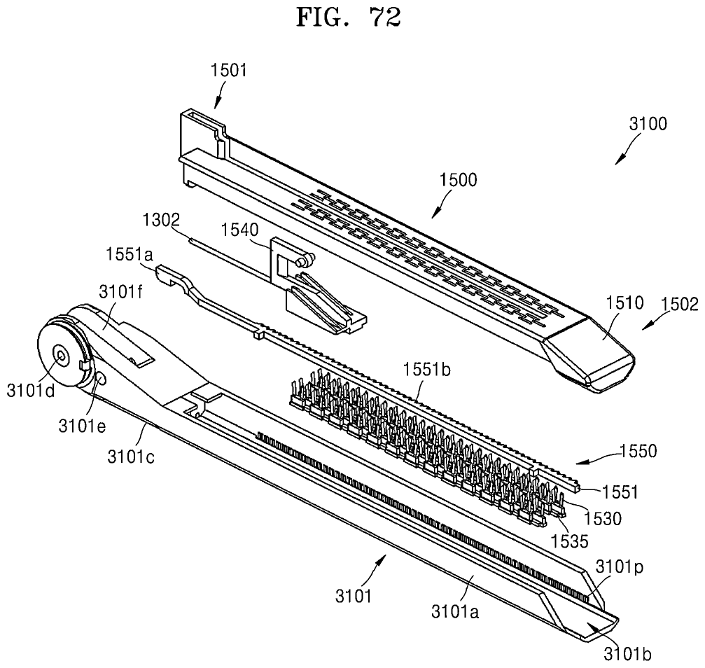

FIG. 72 is a perspective view illustrating a first jaw of the end tool and a cartridge of the surgical instrument according to the second embodiment of the present disclosure.

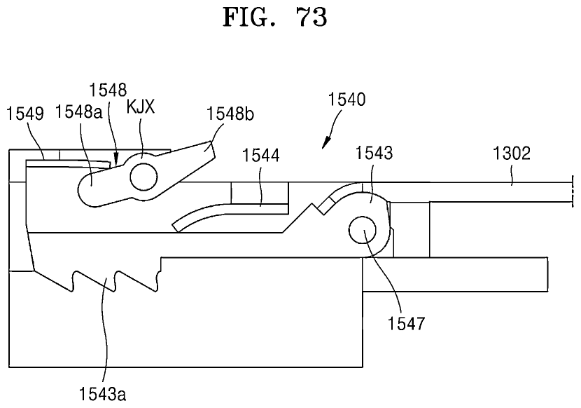

FIG. 73 is a plan view of the operation member of the cartridge of FIG. 72 viewed from one direction.

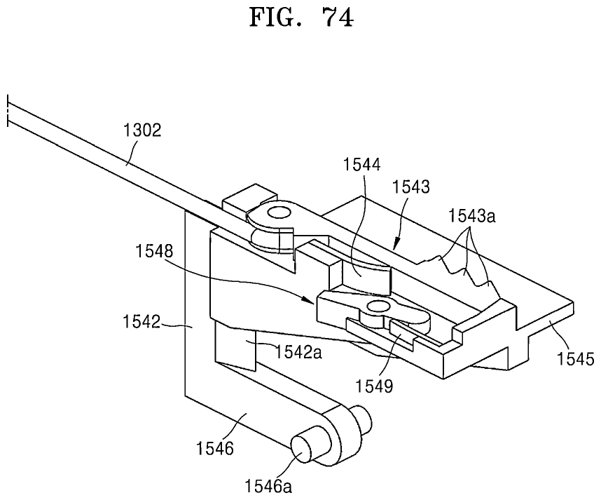

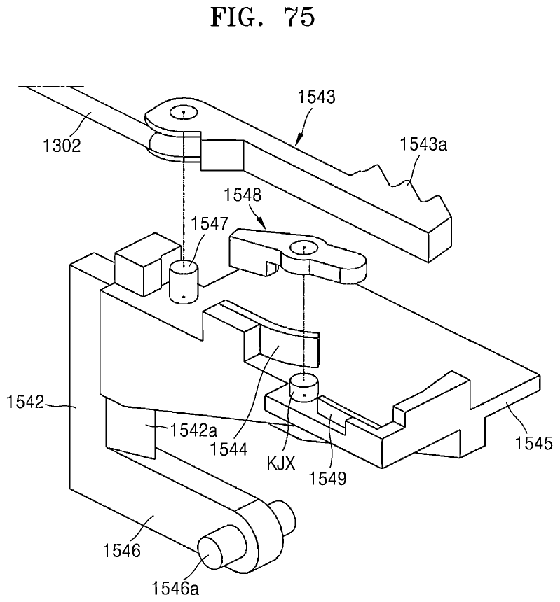

FIGS. 74 and 75 are schematic perspective views of the cartridge of FIG. 72.

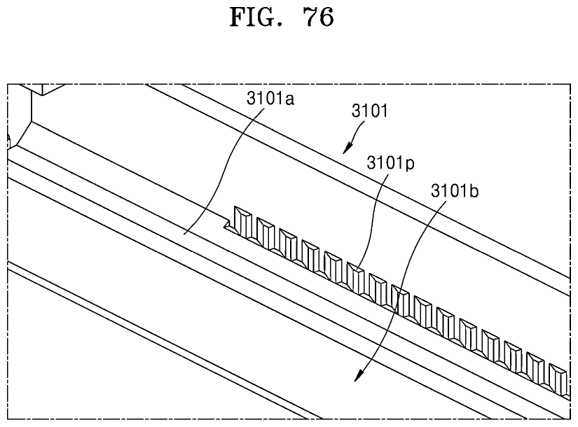

FIG. 76 is a partial perspective view of the first jaw of the cartridge of FIG. 72.

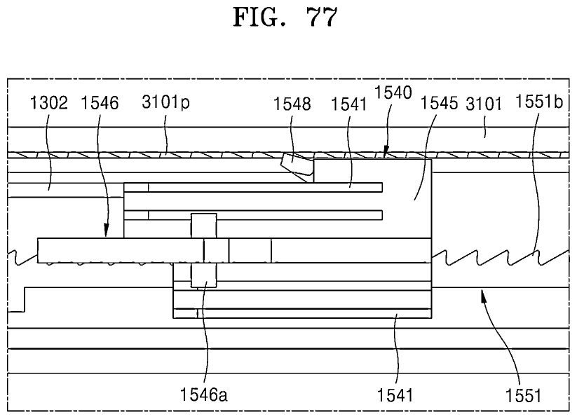

FIG. 77 is a plan view of the cartridge of FIG. 72 viewed from one direction.

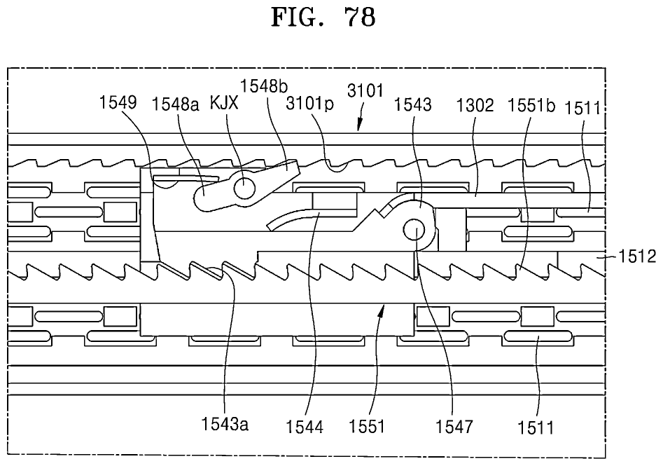

FIG. 78 is a plan view of the cartridge of FIG. 72 viewed from another direction.

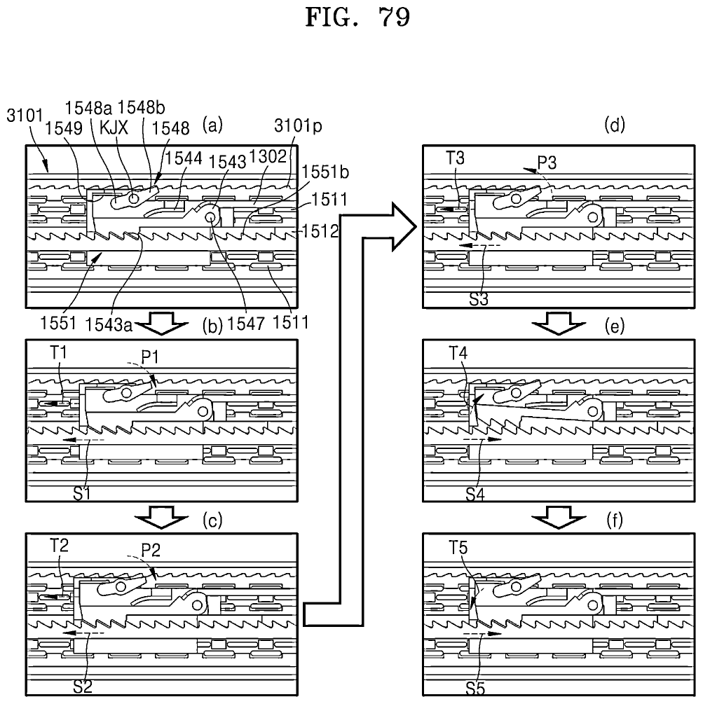

FIG. 79 is a view for describing a backward-movement prevention operation of the operation member of FIG. 72.

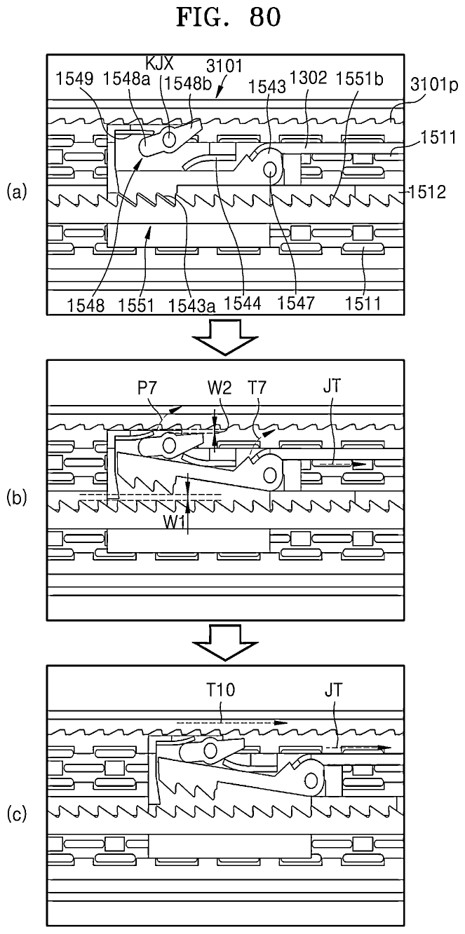

FIG. 80 is a view for describing a backward-movement prevention release and backward movement of the operation member shown in FIG. 72.

Referring to FIG. 72, the end tool 3100 of the second embodiment of the present disclosure includes a pair of jaws for performing a grip motion, and includes a first jaw 3101 and a second jaw (not shown). For convenience of description, the second jaw is omitted here.

In addition, pulleys or the like related to rotational, pitch, and yaw motions of the first jaw 3101 are substantially the same as those described in the first embodiment described above, and thus, detailed descriptions thereof are omitted.

Referring to FIGS. 72 to 80, the first jaw 3101 of the end tool may include a first jaw body 3101a, a cartridge accommodation part 3101b, a guide groove 3101c, a shaft coupling part 3101d, a first jaw coupling part 3101e, and a staple assembly accommodation part 3101f. For convenience of description, differences from the above-described embodiment will be mainly described.

The first jaw 3101 is formed in an overall elongated bar shape, and, for example, the first jaw body 3101a of have a shape similar to an elongated bar. The cartridge 1500 may be accommodated in the first jaw 3101 at a distal end side, and the pulley may be coupled to a proximal end of the first jaw 3101. In other words, the first jaw 3101 may be formed entirely in the form of a hollow box with one surface (upper surface) thereof removed, such that the cartridge accommodation part capable of accommodating the cartridge 1500 may be formed inside the first jaw 3101. That is, the first jaw 3101 may be formed in a substantially “U” shape in cross section.

An engaging part 3101p may be formed on one of inner side surfaces of the first jaw 3101, specifically, on a surface facing recesses 1551b of a reciprocating assembly 1550, and more specifically, on a surface corresponding to a movement path of the operation member 1540 and facing a backward-movement prevention member 1548 of the operation member 1540. In addition, the backward-movement prevention member 1548 of the operation member 1540, which will be described later, may engage with the engaging part 3101p, and through this engagement of the backward-movement prevention member 1548, the backward movement of the operation member 1540 may be prevented.

For example, the engaging part 3101p may have a plurality of grooves and a plurality of protruding regions respectively adjacent to the plurality of grooves, and the backward-movement prevention member 1548 of the operation member 1540 may be formed to engage with the engaging part 3101p, specifically, with the groove of the engaging part 3101p. When the backward-movement prevention member 1548 is engaged with the engaging part 3101p, the backward movement (i.e., the movement toward the proximal end) of the operation member 1540 may be prevented by the backward-movement prevention member 1548 and the engaging part 3101p.

Here, when a reciprocating member 1551 moves backward, only the reciprocating member 1551 may be moved backward and the operation member 1540 may more securely remain stationary in place. That is, the operation member 1540 may be more reliably prevented from moving toward a proximal end of the cartridge 1500 by the backward-movement prevention member 1548 and the engaging part 3101p.

In an optional embodiment, one or more engaging parts 3101p may each have an inclined surface shape, and, for example, the protruding regions and grooves of the engaging part 3101p may be connected by the inclined surfaces. As a specific example, the engaging part 3101p may be formed to have an inclined surface (specifically, the surface on the proximal end side) with a gentle angle and a surface (specifically, the surface on the distal end side) adjacent to the inclined surface and formed vertically or nearly vertically.

The engaging parts 3101p are formed to come into contact with the backward-movement prevention member 1548 of the operation member 1540, thereby preventing the backward movement (i.e., the movement toward the proximal end) of the operation member 1540. This will be described in more detail later.

In the first jaw 3101, the guide groove 3101c configured to guide the movement of the staple link assembly may be formed on one side of the cartridge accommodation part 3101b, for example, on the proximal end side.

Meanwhile, the shaft coupling part 3101d, the first jaw coupling part 3101e, and the staple assembly accommodation part 3101f may be formed on the first jaw 3101 at the proximal end side, and are substantially the same as those described above in the first jaw 2101 of the first embodiment, and thus more detailed descriptions thereof will be omitted.

The second jaw is not illustrated in the drawing but may be applied as is or with modifications within the same or a similar scope as the second jaw of the above-described embodiment (see FIG. 12).

That is, similar to the above-described embodiment, the second jaw may be coupled to the first jaw and maintained in an open state with respect to the first jaw in a standby state through the elastic member. In addition, through the movement of the operation member 1540, a clamp 1546 may be inserted into and engaged with the interior of the second jaw, which causes the second jaw to move closer to the first jaw and maintain a coupled state with the first jaw.

### (Cartridge)

The cartridge 1500 may be disposed in the first jaw 3101, and for example, the cartridge 1500 may be disposed by being coupled to the cartridge accommodation part 3101b of the first jaw 3101. For example, the cartridge 1500 may be integrally formed with the first jaw 3101 while the operation member 1540 is connected to a retraction wire 1302.

In an optional embodiment, the cartridge 1500 may be formed to be mountable to and dismountable from the first jaw 3101. For example, the retraction wire 1302 to be described later and the operation member 1540 may be formed to be couplable to and decouplable from each other, rather than being integrally connected to each other, thereby facilitating the decoupling or coupling of the cartridge 1500 from and to the first jaw 3101.

The cartridge 1500 includes a plurality of staples 1530 and a blade 1542 therein, and performs tissue stapling and cutting. Here, the cartridge 1500 may include a cover 1510, the staples 1530, withdrawal members 1535, the operation member 1540, and the reciprocating assembly 1550.

The cartridge accommodation part of the first jaw 3101 may be formed to accommodate the reciprocating assembly 1550, the operation member 1540, and the staples 1530 therein.

The cover 1510 may be formed to cover an upper portion of the cartridge accommodation part of the first jaw 3101. Staple holes through which the plurality of staples 1530 may be ejected to the outside may be formed in the cover 1510. As the staples 1530, which are accommodated inside the cartridge accommodation part of the first jaw 3101 before a stapling operation, are pushed and raised upward by the operation member 1540 during a stapling motion, and pass through the staple holes of the cover 1510 to be withdrawn to the outside of the cartridge 1500, stapling is performed.

Meanwhile, a slit may be formed in the cover 1510 along a longitudinal direction of the cover 1510. The blade 1542 of the operation member 1540 may protrude out of the cartridge 1500 through the slit. As the blade 1542 of the operation member 1540 passes along the slit, staple-completed tissue may be cut.

The plurality of staples 1530 may be disposed in the cartridge accommodation part of the first jaw 3101. As the operation member 1540 to be described later linearly moves in one direction, the plurality of staples 1530 are sequentially pushed and raised from the inside of the cartridge accommodation part of the first jaw 3101 to the outside, thereby performing suturing, that is, stapling. Here, the staples 1530 may be made of a material that may include titanium, stainless steel, or the like.

Meanwhile, the withdrawal members 1535 may be further disposed between the cartridge accommodation part of the first jaw 3101 and the staples 1530. In other words, it may be described that the staple 1530 is disposed above the withdrawal member 1535. In this case, the operation member 1540 is linearly moved in one direction to push and raise the withdrawal member 1535, and the withdrawal member 1535 may push and raise the staple 1530.

As such, the operation member 1540 may be described as pushing and raising the staples 1530 in both the case in which the operation member 1540 directly pushes and raises the staples 1530 and the case in which the operation member 1540 pushes and raises the withdrawal members 1535 and the withdrawal members 1535 pushes and raises the staples 1530 (i.e., the operation member 1540 indirectly pushes and raises the staples 1530).

The reciprocating assembly 1550 may be disposed on the inner lower side of the cartridge accommodation part 3101b of the first jaw 3101. The reciprocating assembly 1550 may include one or more reciprocating member 1551. In an embodiment, it is illustrated that one reciprocating member 1551 is provided, but in embodiments to be described later, a plurality of reciprocating members 1551 may be provided.

In an embodiment, the reciprocating member 1551 may be a rack. The reciprocating member 1551 may include recesses 1551b and a coupling part 1551a. In detail, the reciprocating member 1551 may be formed in the shape of an elongated bar, and a plurality of recesses 1551b having a sawtooth shape may be formed on one surface thereof. The recess 1551b may be formed to be in contact with the operation member 1540 to be described later, in particular, a ratchet member 1543 of the operation member 1540. In other words, the reciprocating member 1551 may include the plurality of recesses 1551b shaped to engage with ratchets 1543a of the ratchet member 1543.

Here, the reciprocating member 1551 is not fixedly coupled to the other components of the cartridge 1500, and may be formed to be movable relative to the other components of the cartridge 1500.

Meanwhile, in the reciprocating member 1551, the coupling part 1551a may be formed at a proximal end (see 1501 of FIG. 72) side adjacent to the pulley, and the coupling part 1551a may be fastened and coupled to the staple link assembly of the end tool.

The operation member 1540 may be disposed inside the cartridge accommodation part 3101b of the first jaw 3101. The operation member 1540 is formed to be in contact with the reciprocating member 1551, and may be formed to linearly move in one direction in response to a reciprocating linear movement of the reciprocating member 1551.

The operation member 1540 may include a wedge 1541, the blade 1542, the ratchet member 1543, an operation part elastic member 1544, a body 1545, and the clamp 1546.

The body 1545 may be formed in the shape of an elongated square column, and forms a base of the operation member 1540.

The wedge 1541 is formed on at least one side of the body 1545, and may be formed to have a certain inclined surface. That is, the wedge 1541 may be formed to be inclined to a certain extent in the extension direction of the connection part 400. In other words, the wedge 1541 may be formed to have a greater height at the proximal end (see 1501 of FIG. 72) side of the cartridge 1500 than the distal end (see 1502 of FIG. 72) side of the cartridge 1500. In the drawing, it is illustrated that two wedges 1541 are formed on each side of the body 1545, but the concept of the present disclosure is not limited thereto, and the wedge 1541 may be formed in various numbers and shapes depending on the shape of the staple 1530 or the withdrawal member 1535 that is in contact with the wedge 1541.

The wedge 1541 may be formed to be in contact with the withdrawal members 1535 or the plurality of staples 1530 in turn and may serve to sequentially push and raise the staples 1530. The operation member 1540 may serve to withdraw the staples 1530 to the outside of the cartridge 1500 by sequentially pushing and raising the staples 1530 while moving toward the distal end.

The blade 1542 may be formed on one side of the wedge 1541, more specifically, on the proximal end 1501 side of the wedge 1541. An edge 1542a formed to be sharp to cut tissue is formed in one region of the blade 1542. As at least a portion of the edge 1542a is withdrawn to the outside of the first jaw 3101 and the cartridge 1500, tissue disposed between the first jaw 3101 and the second jaw may be cut. The edge 1542a of the blade 1542 may be always withdrawn to the outside of the first jaw 3101. Alternatively, the edge 1542a of the blade 1542 may normally be accommodated inside the first jaw 3101 or inside the cartridge 1500, and may be withdrawn to the outside of the first jaw 3101 only when the operation member 1540 is moved in a longitudinal direction.

The ratchet member 1543 is formed on one side of the wedge 1541, more specifically, below wedge 1541, and may be formed to face the reciprocating member 1551 to be described later. The ratchet member 1543 may be formed in the shape of a bar and may include a plurality of ratchets 1543a on one surface. The operation member 1540 is moved only in one direction (i.e., toward the distal end) with respect to the reciprocating member 1551 by the ratchet member 1543. The ratchets 1543a of the ratchet member 1543 may be formed to be in contact with the recess 1551b of the reciprocating member 1551 described above. Meanwhile. the ratchet member 1543 may be formed to rotationally move around a rotation shaft 1547. For example, the ratchet member 1543 may be formed to rotationally move by being inserted into the rotation shaft 1547 formed in a protruding shape on the body 1545 of the operation member 1540. The rotation shaft 1547 may be disposed closer to the proximal end 1501 than the plurality of ratchets 1543a.

Meanwhile, the retraction wire 1302 may be connected to the ratchet member 1543, and by pulling the retraction wire 1302, the ratchet member 1543 may rotationally move around the rotation shaft 1547. As a specific example, the retraction wire 1302 may be connected to one region around the rotation shaft 1547. In addition, when a greater force is applied to pull the retraction wire 1302, the retraction wire 1302 may move backward, and the operation member 1540 connected to the retraction wire 1302 may also move backward (toward the proximal end).

The operation part elastic member 1544 is formed on one side of the body 1545 or the wedge 1541 and serves to apply a certain elastic force to the ratchet member 1543. As an example, the operation part elastic member 1544 may be formed such that one region is in contact with the body 1545, and another region that is different from the one region is in contact with the ratchet member 1543. The operation part elastic member 1544 may apply an elastic force in a direction in which the ratchet member 1543 comes into close contact with the reciprocating member 1551. To this end, the operation part elastic member 1544 may be formed in the form of a leaf spring, and may be provided in various forms capable of providing a certain elastic force to the ratchet member 1543, such as a coil spring, a dish spring, and the like. In addition, although not shown in the drawings, in another optional embodiment, the operation part elastic member 1544 may be integrally formed with the operation member 1540.

### <Backward-Movement Prevention Member>

The backward-movement prevention member 1548 may be formed in a manner that allows a rotational motion, such that one region thereof engages with the engaging part 3101p of the first jaw 3101, and, when at least the operation member 1540 is subjected to a backward force, i.e., a force toward the proximal end, the backward-movement prevention member 1548 may engage with the engaging part 3101p to prevent the backward movement of the operation member 1540.

For example, the backward-movement prevention member 1548 may be formed to perform a rotational motion around a backward-movement prevention rotation shaft KJX formed on one side of the operation member 1540, and a first side 1548a (on the distal end side) and a second side 1548b (on the proximal end side) may be formed around the backward-movement prevention rotation shaft KJX. The backward-movement prevention member 1548 may perform a rotational motion around the backward-movement prevention rotation shaft KJX, and may rotate in a direction that allows the second side 1548b (on the proximal end side) to come into contact and engage with the engaging part 3101p of the first jaw 3101, or in the opposite direction to move away from the engaging part 3101p.

A backward-movement prevention elastic member 1549 may be formed on one side of the body 1545 or the wedge 1541 and may apply a certain elastic force to the first side 1548a (on the distal end side) of the backward-movement prevention member 1548. The backward-movement prevention elastic member 1549 may apply an elastic force in a direction that brings the first side 1548a (on the distal end side) closer to the ratchet member 1543. Through this, the second side 1548b, which is opposite to the first side 1548a, may be subjected to a force in a direction away from the ratchet member 1543, i.e., in a direction closer to the engaging part 3101p of the first jaw 3101. The backward-movement prevention elastic member 1549 may effectively prevent the backward-movement prevention member 1548 from losing its functionality because the first side 1548a of the backward-movement prevention member 1548 excessively moves away from the ratchet member 1543. As a result, when a certain force or more is applied to the first side 1548a, causing the first side 1548a to move closer and engage with the engaging part 3101p of the first jaw 3101, the backward movement of the operation member 1540 can be reliably prevented through the engagement of the backward-movement prevention member 1548 and the engaging part 3101p.

The clamp 1546 may be formed on one side of the blade 1542 and may be formed in a shape that is approximately parallel to the body 1545 or the wedge 1541. In addition, a protrusion 1546a may be formed at one end portion of the clamp 1546, and the protrusion 1546a may move along a passageway (see 2102d of FIG. 18) of the second jaw. The clamp 146 may be coupled to the second jaw and, as a specific example, brought into the interior of the second jaw through the guide part of the second jaw, and as the clamp 1546 moves forward, the first jaw 3101 and the second jaw may naturally maintain a closed state.

Here, as an example, the clamp 1546 may be formed parallel to the wedge 1541 and configured to have an overall ‘U’ shape, thereby better withstanding a strong pressure applied to the wedge 1541 during the stapling motion.

Here, a first surface with a gentle slope (the surface on the distal end side) and a second surface that is vertical or nearly vertical (the surface on the proximal end side) may be formed on the ratchet 1543a of the ratchet member 1543, and since details thereof are substantially the same as those described in the ratchet member 543 in the first embodiment described above, detailed descriptions are omitted.

In addition, to engage with the ratchet 1543a of the ratchet member 1543, the recess 1551b of the reciprocating member 1551 may also include a first surface with a gentle slope and a second surface that is vertical or nearly vertical, and since details thereof are substantially the same as those described in the reciprocating member 551 of the first embodiment described above, detailed descriptions are omitted.

In a state in which the reciprocating member 1551 and the ratchet member 1543 are coupled to each other (or engaged or in close contact with each other), the reciprocating member 1551 and the ratchet member 1543 may act as a kind of ratchet, allowing movement only in one direction.

As an example, assuming that the reciprocating member 1551 is fixed, in a state in which the reciprocating member 1551 and the ratchet member 1543 are coupled to each other (or engaged or in close contact with each other), when the reciprocating member 1551 moves toward the distal end 1502, the ratchet member 1543 is also moved toward the distal end 1502 by the reciprocating member 1551.

In contrast, when the reciprocating member 1551 is moved toward the proximal end 1501 in a state in which the reciprocating member 1551 and the ratchet member 1543 are coupled to each other (or engaged or in close contact with each other), only the reciprocating member 1551 is moved alone toward the proximal end 1501 while the ratchet member 1543 a fixed.

### <Backward-Movement Prevention Operation>

Meanwhile, in the present embodiment, the backward-movement prevention member 1548 may effectively prevent the backward movement of the operation member 1540 and, specifically, may prevent the operation member 1540 from moving together with the reciprocating member 1551 in the direction of the proximal end 1501.

FIG. 79 is a plan view illustrating the backward-movement prevention operation of the operation member of the end tool of FIG. 72.

As shown in FIG. 79B, when the reciprocating member 1551 moves in the direction of the distal end 1502 (in the direction of an arrow S1), the ratchet member 1543 is pressed and also moved in the direction of the arrow S1, and the operation member 1540 is also moved in the same direction, i.e., in the direction of an arrow T1. At this time, the backward-movement prevention member 1548 may rotationally move around in the direction of an arrow P1 around the backward-movement prevention rotation shaft KJX. For example, during the forward movement of the operation member 1540 (in the T1 direction), the backward-movement prevention member 1548 may rotationally move in the P1 direction, which is a direction away from the engaging part 3101p of the first jaw 3101, by the engaging part 3101p of the first jaw 3101, and through this, the second side 1548b disengages from the engaging part 3101p of the first jaw 3101, for example, a region including an end portion of the second side 1548b moves out of a groove region of the engaging part 3101p, which corresponds to the region including the end portion of the second side 1548b, as shown in FIG. 79A. In addition, the forward movement of the operation member 1540, i.e., in the S1 direction, is not affected.

As shown in FIG. 79C, when the reciprocating member 1551 moves further in the direction of the distal end 1502 (in the direction of an arrow S2), the operation member 1540 also moves further in the same direction, i.e., in the direction of an arrow T2. At this time, the backward-movement prevention member 1548 further rotates around the backward-movement prevention rotation shaft KJX in the direction of an arrow P2, and the second side 1548b is in a state of not being engaged with the engaging part 3101p of the first jaw 3101. In addition, the forward movement of the operation member 1540, i.e., in the S2 direction, is not affected. That is, the second side 1548b corresponds to the protruding region of the engaging part 3101p of the first jaw 3101, and the backward-movement prevention member 1548 may remain in the state of not being engaged with the engaging part 3101p of the first jaw 3101.

As shown in FIG. 79D, when the reciprocating member 1551 moves further in the direction of the distal end 1502 (in the direction of an arrow S3), the operation member 1540 also moves further in the same direction, i.e., in the direction of an arrow T3. At this time, the second side 1548b may be in a state of being engaged with the engaging part 3101p of the first jaw 3101 through the forward movement of the backward-movement prevention member 1548. That is, as the first side 1548a receives an elastic force from the backward-movement prevention elastic member 1549, the second side 1548b, which is a side opposite to the first side 1548a, may rotate in the P3 direction toward the engaging part 3101p and engage with the groove of the engaging part 3101p in a corresponding state. In this case, when the reciprocating member 1551 moves further in the forward direction (in the T3 direction), the second side 1548b may rotationally move again in a direction away from the engaging part 3101p of the first jaw 3101, thereby disengaging from the engaging part 3101p of the first jaw 3101 without affecting the forward movement. Conversely, as shown in FIG. 79E, when the reciprocating member 1551 moves in the direction of the proximal end 1501 (in the direction of an arrow S4), the second side 1548b may remain engaged with the engaging part 3101p of the first jaw 3101. For example, the second side 1548b may be caught on the inclined surface of the engaging part 3101p, and as a specific example, the second side 1548b may be caught by the vertical or nearly vertical surface among the two inclined surfaces forming the groove and the protruding region of the engaging part 3101p.

At this time, when the reciprocating member 1551 moves backward in the S4 direction, the ratchet member 1543 of the operation member 1540 may also rotationally move (in the T4 direction), thereby releasing the ratchet coupling with the reciprocating member 1551, and in this case, the release may not be complete, but a slight connection may remain, which could undesirably cause the operation member 1540 to move backward (i.e., move toward the proximal end) along with the reciprocating member 1551. In an embodiment, the second side 1548b of the backward-movement prevention member 1548 remains engaged with the engaging part 3101p of the first jaw 3101, thereby preventing the backward movement of the operation member 1540, maintaining the stationary state, and as a result, implementing the prevention of backward movement of the operation member 1540. As shown in FIG. 79F, as the reciprocating member 1551 moves backward while the operation member 1540 remains stationary (with only the ratchet member 1543 rotating in the T5 direction), the ratchet member 1543 may re-engage with the reciprocating member 1551, and when the reciprocating member 1551 moves further backward, the state as shown in FIG. 79E is restored, thereby enabling the implementation of the backward-movement prevention operation.

### <Backward Movement>

Meanwhile, in the present disclosure, the retraction wire 1302 is provided to enable the backward movement of the operation member 1540, i.e., the movement of the operation member 1540 toward the proximal end (1501 of FIG. 72).

FIG. 80 is a plan view illustrating the backward movement of the operation member of the end tool of FIG. 72.

As described above, the ratchet member 1543 may be formed to be rotatable around the rotation shaft 1547, and as a specific example, the ratchet member 1543 may be inserted into the rotation shaft 1547 formed in a shape protruding from the body 1545 of the operation member 1540 and may rotationally move around the rotation shaft 1547.

The retraction wire 1302 may be connected to the ratchet member 1543, and by pulling the retraction wire 1302, the ratchet member 1543 may rotationally move around the rotation shaft 1547. As a specific example, the retraction wire 1302 may be coupled to one region around the rotation shaft 1547.

When the retraction wire 1302 is pulled in the direction of an arrow JT of FIG. 80B, the ratchet member 1543 is rotated around the rotation shaft 1547 in the direction of an arrow T7 of FIG. 80B.

Here, when a certain amount or more of force is applied to the retraction wire 1302, the ratchet member 1543 is spaced away from the reciprocating member 1551 to generate a separation gap W1, and the coupling (engagement) between the ratchet member 1543 and the reciprocating member 1551 is released, so that the operation member 1540 becomes movable with respect to the reciprocating member 1551.

At this time, when the ratchet member 1543 rotates in the direction of the arrow T7, one region of the ratchet member 1543 comes into contact with the backward-movement prevention member 1548, and when ratchet member 1543 further rotates, a force is applied to the first side 1548a (on the distal end side) of the backward-movement prevention member 1548. Accordingly, the backward-movement prevention member 1548 rotationally moves around the backward-movement prevention rotation shaft KJX, which causes the second side 1548b opposite to the first side 1548a to rotate in a P7 direction away from the engaging part 3101p of the first jaw 3101, thereby releasing the engagement between the second side 1548b and the engaging part 3101p and generating a gap W2 therebetween.

In this state, when the retraction wire 1302 is further pulled in the direction of an arrow JT of FIG. 80C, the operation member 1540 as a whole is moved in the direction of an arrow T10 of FIG. 80C in a state in which the ratchet member 1543 is spaced away from the reciprocating member 1551.

As a result, the operation member 1540 can be moved backward, i.e., can be moved toward the proximal end 3105 by the retraction wire 1302.

As a result, during the stapling process, in which the reciprocating member 1551 of the cartridge 1500 repeatedly moves forward and backward, when the reciprocating member 1551 moves forward, the operation member 1540 moves forward together with the reciprocating member 1551, and when the reciprocating member 1551 moves backward, only the reciprocating member 1551 moves backward, and the operation member 1540 remains stationary in place without moving backward. As the operation member 1540 moves forward while repeating this process, the staple 1530 may be stapled by the wedge 1541 while the blade 1542 cuts the stapled tissue.

In addition, when the operation member 1540 needs to be moved backward again toward the proximal end, the operation member 1540 may be easily moved backward by pulling the operation member 1540 through the retraction wire 1302.

### Third Embodiment of Surgical Instrument

Hereinafter, an end tool 4100 of a surgical instrument according to a third embodiment of the present disclosure will be described. Here, the end tool 4100 of the surgical instrument according to the third embodiment of the present disclosure is different from the end tool (see 2100 of FIG. 2 or the like) of the surgical instrument according to the first embodiment of the present disclosure described above in that a configuration of a cartridge 4500 including an operation member 4540, a backward-movement prevention release wire 4303, and the like is changed. Hereinafter, the configuration that is different from that of the first embodiment will be described in detail.

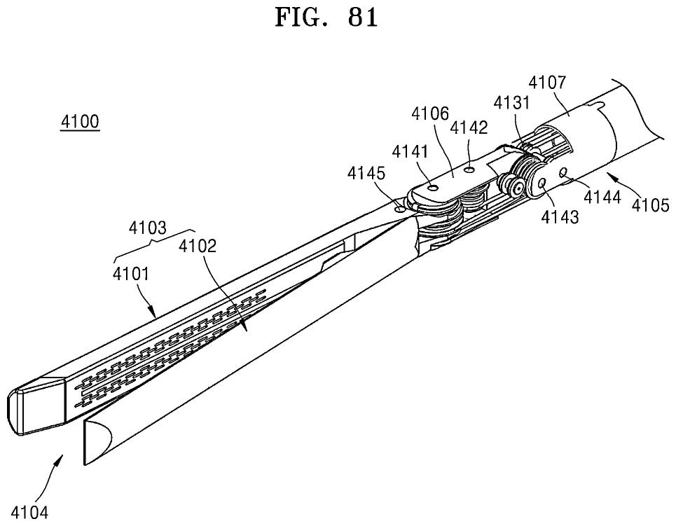

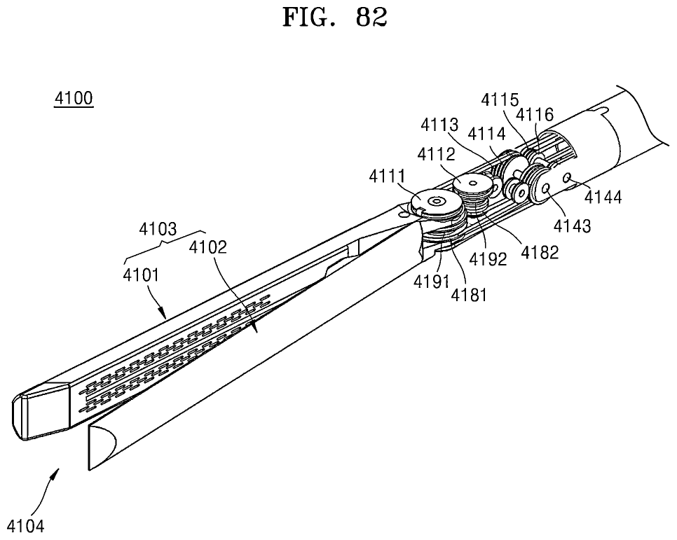

FIGS. 81 and 82 are perspective views illustrating the end tool of the surgical instrument according to the third embodiment of the present disclosure.

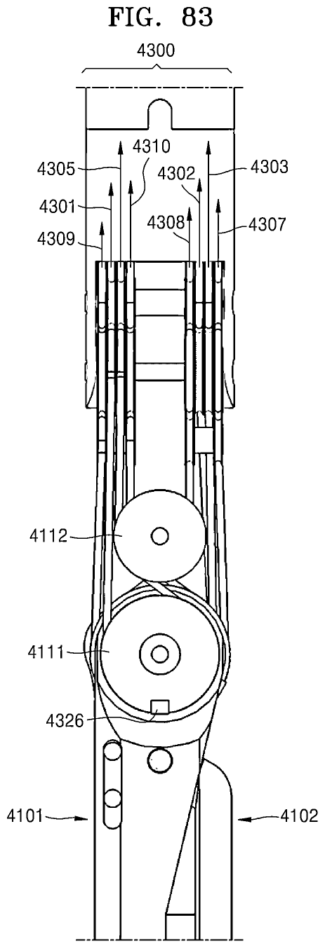

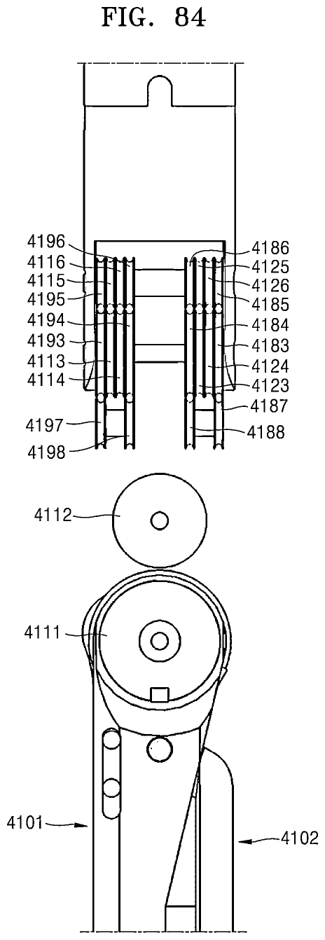

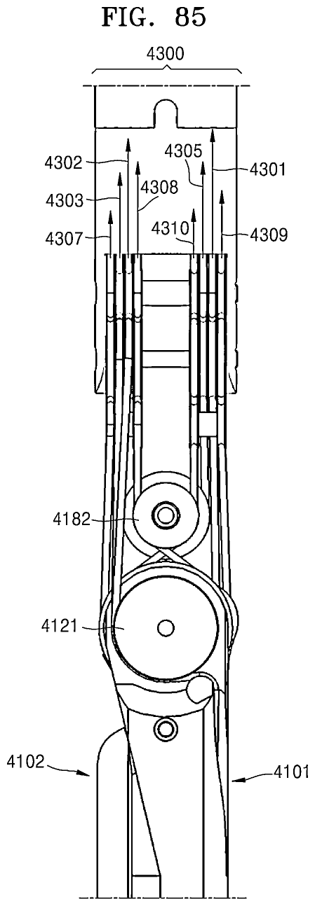

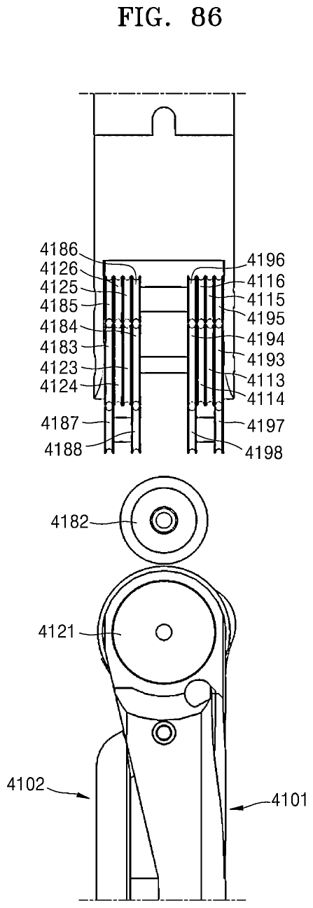

FIGS. 83 to 86 are plan views illustrating the end tool of the surgical instrument of FIG. 81.

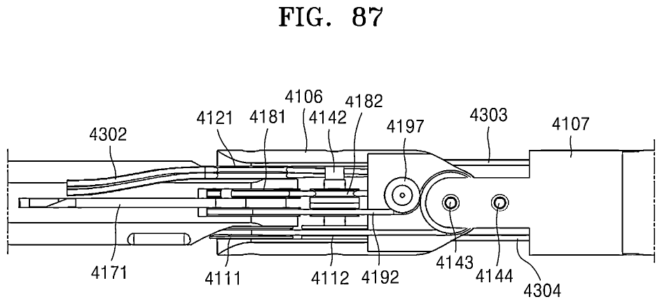

FIG. 87 is a side view illustrating the end tool of the surgical instrument of FIG. 81.

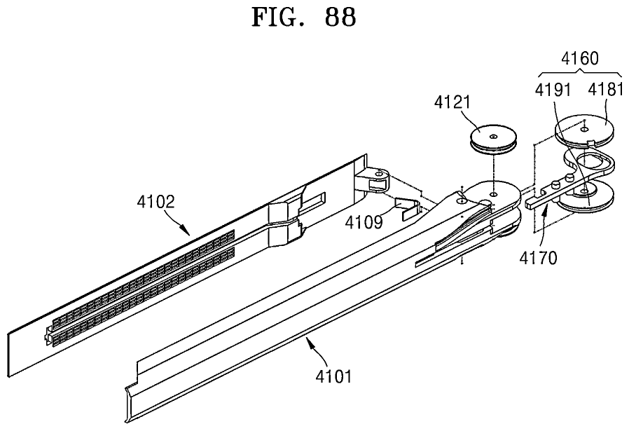

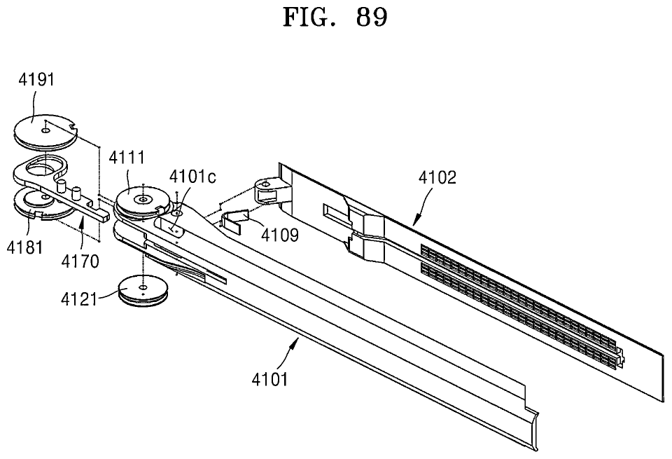

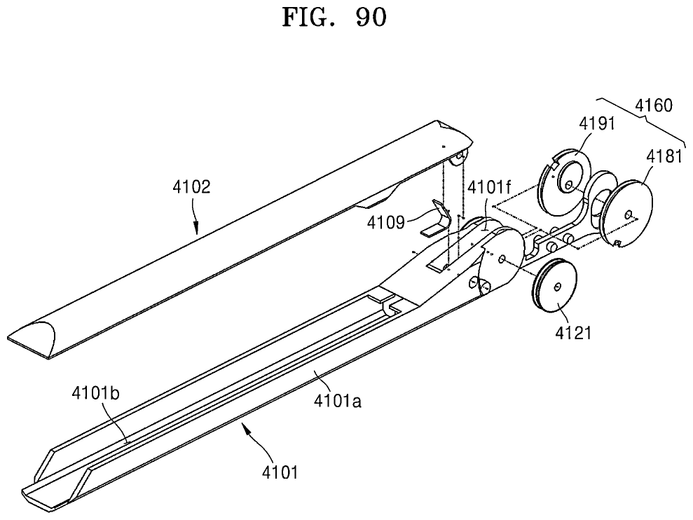

FIGS. 88 to 90 are exploded perspective views of the end tool of the surgical instrument of FIG. 81.

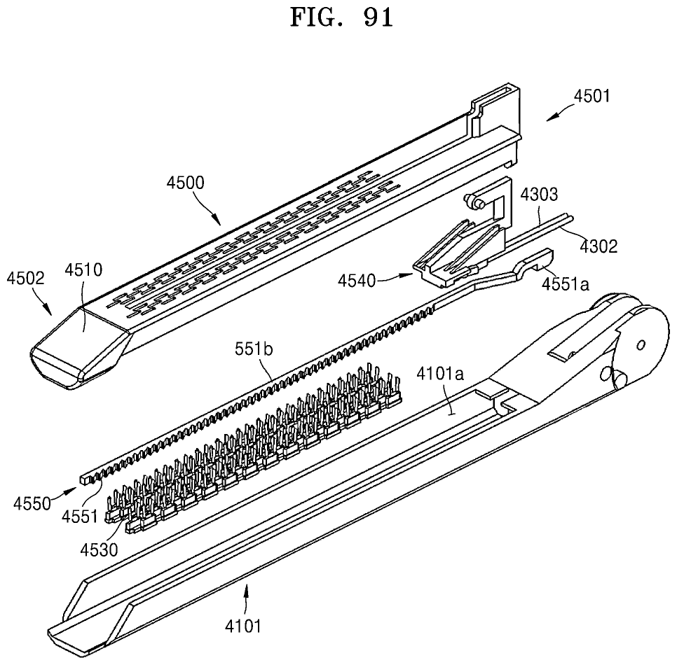

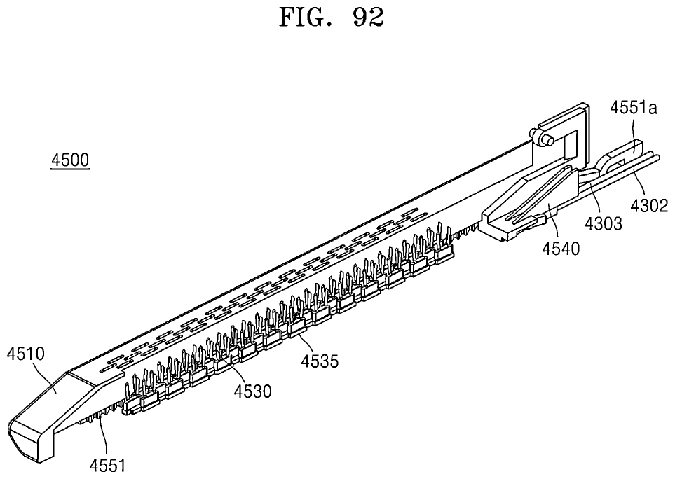

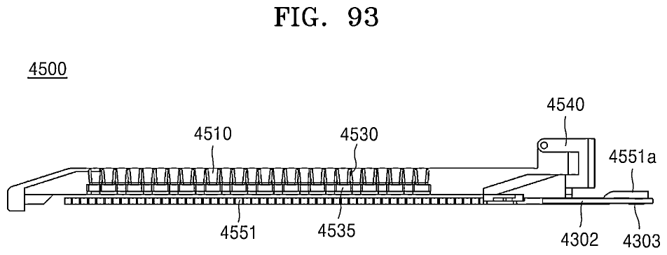

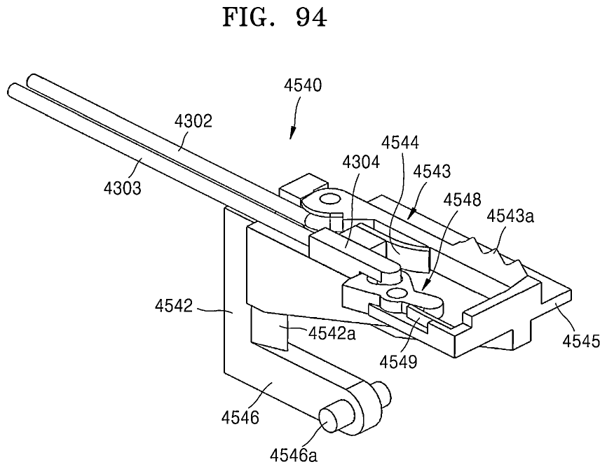

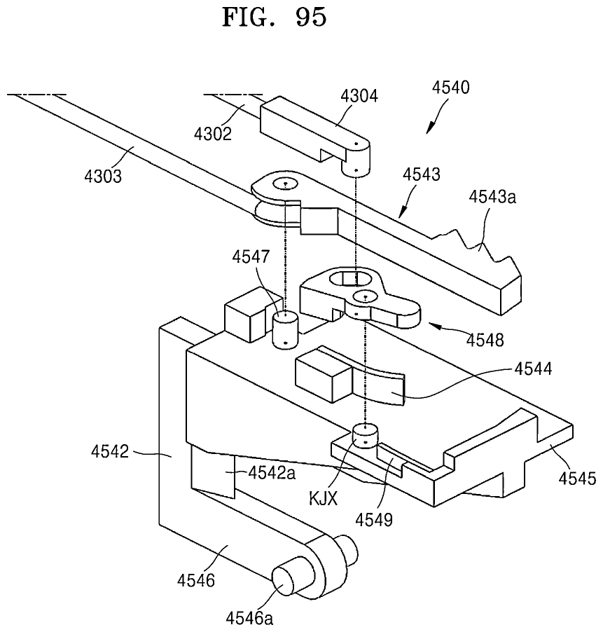

FIG. 91 is a perspective view illustrating a first jaw and a cartridge of the surgical instrument of FIG. 81. FIG. 92 is an exploded perspective view illustrating the cartridge of FIG. 91, and FIG. 93 is a side cross-sectional view illustrating the cartridge of FIG. 91. FIGS. 94 and 95 are perspective views of an operation member of the cartridge of FIG. 91 viewed from one direction.

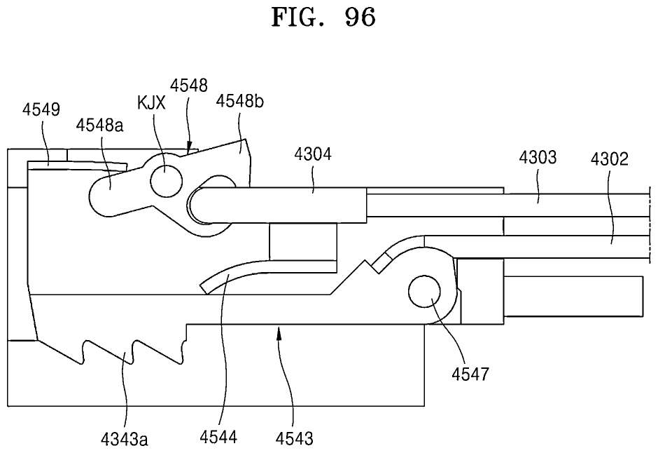

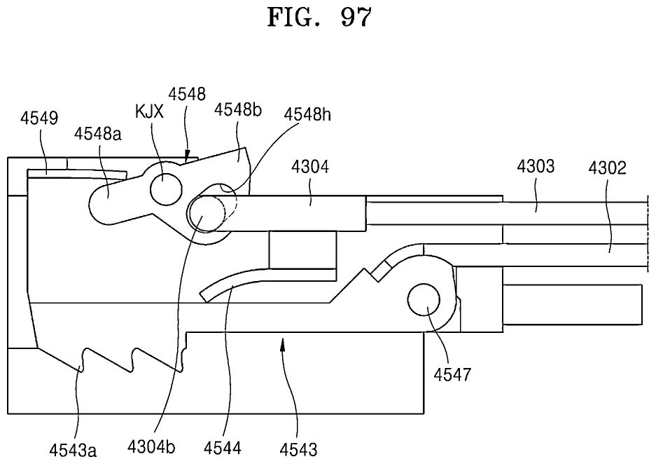

FIGS. 96 and 97 are plan views of the operation member of the cartridge of FIG. 91 viewed from one direction.

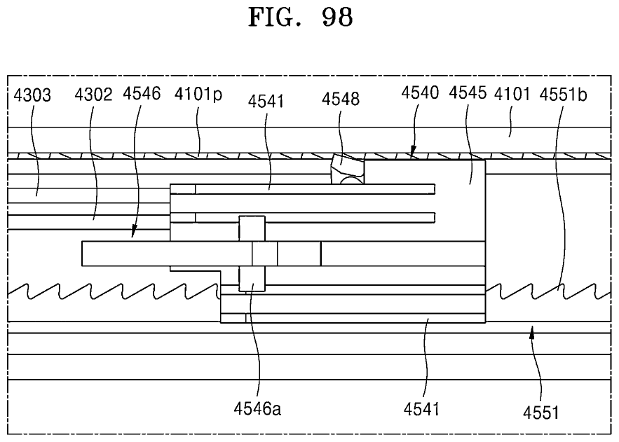

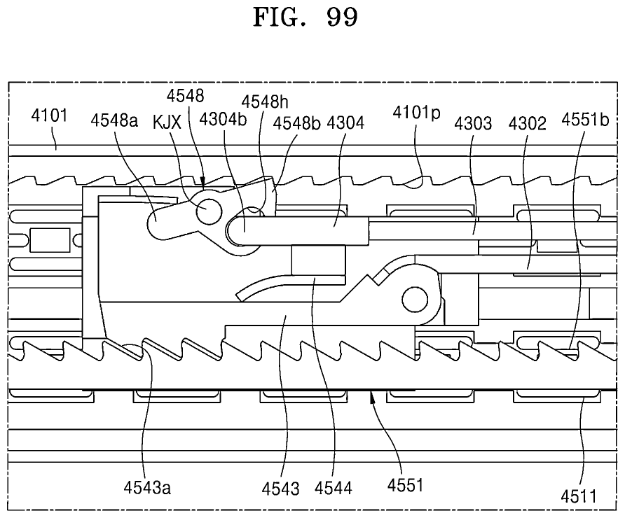

FIGS. 98 and 99 are plan views of the cartridge of FIG. 91 viewed from one direction and another direction different from the one direction, respectively.

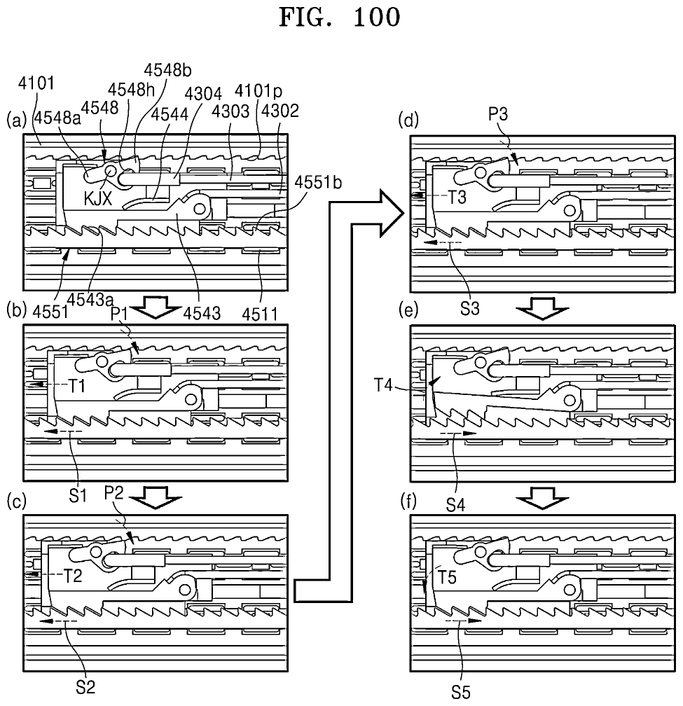

FIG. 100 is a plan view illustrating a backward-movement prevention operation of the operation member of the end tool of FIG. 91.

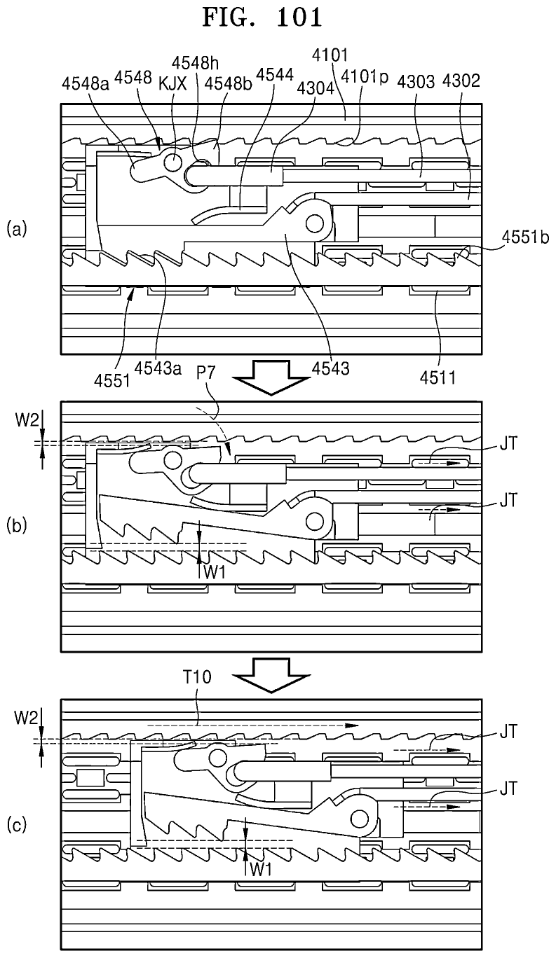

FIG. 101 is a plan view illustrating a backward movement of the operation member of the end tool of FIG. 91.

Referring to FIGS. 81 to 101, the end tool 4100 of the third embodiment of the present disclosure includes a pair of jaws 4103 for performing a grip motion, that is, a first jaw 4101 and a second jaw 4102. Here, each of the first jaw 4101 and the second jaw 4102, or a component encompassing the first jaw 4101 and the second jaw 4102 may be referred to as the jaw.

Meanwhile, the end tool 4100 includes a plurality of pulleys including a pulley 4111, a pulley 4112, a pulley 4113, a pulley 4114, a pulley 4115, and a pulley 4116 that are related to a rotational motion of the jaw 4103. The pulleys related to the rotational motion of the jaw 4203 described in the present embodiment are substantially the same as the pulley 2111, the pulley 2112, the pulley 2113, the pulley 2114, the pulley 2115, and the pulley 2116 described in the first embodiment, and thus, a detailed description thereof will be omitted herein.

In addition, the end tool 4100 may include a pulley 4121, a pulley 4122, and a pulley 4123 that are related to a rotational movement of a retraction wire 4302.

In addition, the end tool 4100 may include may include one or more pulleys related to the rotational motion of the backward-movement prevention release wire 4303, for example, a pulley 4124 and a pulley 4126.

Meanwhile, the pulley 4121 has two grooves formed therein, so that at least one region of the retraction wire 4302 may be wound in one groove and at least one region of the backward-movement prevention release wire 4303 may be wound in another groove.

In addition, in an optional embodiment, the retraction wire 4302 and the backward-movement prevention release wire 4303 may be wound around different pulleys, respectively.

Further, the end tool 4100 of the third embodiment of the present disclosure may include a rotation shaft 4141, a rotation shaft 4242, a rotation shaft 4143, and a rotation shaft 4144. Here, the rotation shaft 4141 and the rotation shaft 4142 may be inserted through an end tool hub 41406, and the rotation shaft 4143 and the rotation shaft 4144 may be inserted through a pitch hub 4107. The rotation shaft 4141, the rotation shaft 4142, the rotation shaft 4143, and the rotation shaft 4144 may be arranged sequentially from a distal end 4104 toward a proximal end 4105 of the end tool 4100.

In addition, the end tool 4100 of the third embodiment of the present disclosure may include the end tool hub 4106 and the pitch hub 4107.

The rotation shaft 4141 and the rotation shaft 4142 may be inserted through the end tool hub 4106, and the pulley 4111 and the pulley 4112 axially coupled to the rotation shaft 4141 and at least some of the first jaw 4201 and the second jaw 4202 coupled to the pulley 4111 and the pulley 4112 may be accommodated inside the end tool hub 4106.

The rotation shaft 4143 and the rotation shaft 4144 may be inserted through the pitch hub 4107, and the pitch hub 4207 may be axially coupled to the end tool hub 4106 by the rotation shaft 4143. Accordingly, the end tool hub 4106 may be formed to be pitch-rotatable around the rotation shaft 4143 with respect to the pitch hub 4107.

Meanwhile, the end tool 4100 of the third embodiment of the present disclosure may further include components, such as a staple drive assembly 4150 including a staple pulley assembly 4160 and a staple link assembly 4170, to perform stapling and cutting motions.

The staple pulley assembly 4160 may include one or more staple pulleys. The staple pulley assembly 4160 may be formed between the pulley 4111 and the pulley 4121 to be adjacent to the pulley 4111 and the pulley 4121. In an embodiment, it is assumed that the staple pulley assembly 4160 includes two pulleys that are a first staple pulley 4181 and a second staple pulley 4191.

The staple link assembly 4170 may include one or more link members 4171. In addition, the link member 4171 may include one or more links. In the third embodiment of the present disclosure, it is assumed that the staple link assembly 4170 includes one link member 4171, and the link member 4171 includes one link.

Meanwhile, in the third embodiment of the present disclosure, components for performing the stapling and cutting motions are substantially the same as those described in the first embodiment, and thus detailed descriptions thereof will be omitted herein.

### <First Jaw and Cartridge>

Referring to FIGS. 81 to 91 and the like, the first jaw 4101 of the end tool may include a first jaw body 4101a, a cartridge accommodation part 4101b, a guide groove 4101c, a shaft coupling part 4101d, a first jaw coupling part 4101e, and a staple assembly accommodation part 4101f.

The first jaw 4101 is formed in an overall elongated bar shape, and, for example, the first jaw body 4101a may have a shape similar to an elongated bar. The cartridge 4500 may be accommodated in the first jaw 4101 at a distal end side, and the pulley 4111 may be coupled to a proximal end of the first jaw 4101. In other words, the first jaw 4101 may be formed entirely in the form of a hollow box with one surface (upper surface) thereof removed, such that the cartridge accommodation part capable of accommodating the cartridge 4500 may be formed inside the first jaw 4101. That is, the first jaw 4101 may be formed in an approximately “U” shape in cross section.

Here, the first jaw 4101 and the pulley 2111 may be formed integrally, or may be formed as separate members and then coupled to each other.

An engaging part 4101p (see FIG. 99) may be formed on one of inner side surfaces of the first jaw 4101, specifically, on a surface facing recesses 4551b of a reciprocating assembly 4550, and more specifically, on a surface corresponding to a movement path of the operation member 4540 and facing a backward-movement prevention member 4548 of the operation member 4540. In addition, the backward-movement prevention member 4548 of the operation member 4540, which will be described later, may engage with the engaging part 4101p, and through this engagement of the backward-movement prevention member 4548, the backward movement of the operation member 4540 may be prevented.

For example, the engaging part 4101p may have a plurality of grooves and a plurality of protruding regions and may be formed to engage with the backward-movement prevention member 4548 of the operation member 4540. When the backward-movement prevention member 4548 is engaged with the engaging part 4101p, the backward movement (i.e., the movement toward the proximal end) of the operation member 4540 may be prevented by the backward-movement prevention member 4548 and the engaging part 4101p.

Here, when a reciprocating member 4551 moves backward, only the reciprocating member 4551 may be moved backward and the operation member 4540 may more securely remain stationary in place. That is, the operation member 4540 may be more reliably prevented from moving toward a proximal end of the cartridge 4500 by the backward-movement prevention member 4548 and the engaging part 4101p.

In an optional embodiment, one or more of the engaging parts 4101p may each have an inclined surface shape. For example, the engaging part 4101p may be formed to have an inclined surface (specifically, the surface on the proximal end side) with a gentle angle and a surface (specifically, the surface on the distal end side) adjacent to the inclined surface and formed vertically or nearly vertically.

The engaging parts 4101p are formed to come into contact with the backward-movement prevention member 4548 of the operation member 4540, thereby preventing the backward movement (i.e., the movement toward the proximal end) of the operation member 4540. This will be described in more detail later.

In the first jaw 4101, the guide groove 4101c configured to guide the movement of the staple link assembly may be formed on one side of the cartridge accommodation part 4101b, for example, on the proximal end side.

Meanwhile, the shaft coupling part 4101d, the first jaw coupling part 4101e, and the staple assembly accommodation part 4101f may be formed on the proximal end side of the first jaw 4101. The first jaw coupling part 4101e is formed in the shape of a hole, and a rotation shaft 4145, which is a jaw rotation shaft, may correspond to the first jaw coupling part 4101e.

The second jaw 4102 may include a second jaw body 4102a, a second jaw coupling part 4102b, a guide part 4102c, and a passageway.

The second jaw 4102 is formed in an overall elongated bar shape, and, for example, the second jaw body 4102a may be formed in a bar shape to correspond to the first jaw 4101 in at least one region. An anvil is formed in the second jaw 4102 at the distal end side, and the second jaw coupling part 4102b is formed on the proximal end to be coupled to the first jaw 4101, thereby allowing the second jaw 4102 to rotate around the rotation shaft 4145.

In detail, among surfaces of the second jaw 4102, the surface facing the first jaw 4101 may have the anvil formed thereon, and the anvil may be formed in a flat plane shape with shapes corresponding to the shape of the staple 4530 formed on one surface thereof. The anvil of the second jaw 4102 may serve as a support for supporting the staple 4530 on the opposite side of the operation member 4540 when the operation member 4540 pushes and raises the staple 4530 during a stapling motion, so that the staple 4530 is bent.

Meanwhile, an elastic member 4109 may be disposed between the first jaw 4101 and the second jaw 4102. The elastic member 4109 may be formed and disposed to apply a force in a direction in which the first jaw 4101 and the second jaw 4102 move away from each other. For example, the elastic member 4109 may have a plate shape and a curved form that splits toward both sides in the direction of the distal end of the end tool 4100.

The elastic member 4109 may be disposed adjacent to the first jaw coupling part 4101e and the second jaw coupling part 4102b. For example, at least one region of the elastic member 4109 may be accommodated in the staple assembly accommodation part 4101f of the first jaw 4101, while another region of the elastic member 4109 is accommodated in a groove adjacent to the second jaw coupling part 4102b of the second jaw 4102.

The first jaw coupling part 4101e may be formed relatively closer to the distal end side of the first jaw 4101 than the shaft coupling part 4101d. The first jaw coupling part 4101e is formed in the shape of a hole, and the rotation shaft 4145 may correspond to the first jaw coupling part 4101e. The first jaw coupling part 4101e and the second jaw coupling part 4102b are axially coupled by the rotation shaft 4145, so that the second jaw 4102 can rotationally move relative to the first jaw 4101. Through this, the first jaw 4101 and the second jaw 4102 can perform opening and closing motions, such as an actuation motion.

The staple assembly accommodation part 4101f may be disposed closer to the proximal end of the body 4101a of the first jaw 4101 than the cartridge accommodation part 4101b. The staple assembly accommodation part 4101f may have a groove shape to accommodate one region of the second jaw 4102, for example, the second jaw coupling part 4102b. In addition, the staple assembly accommodation part 4101f may be formed to accommodate at least a portion of the staple drive assembly therein. For example, at least some of the first staple pulley 4181, the second staple pulley 4191, and the staple link assembly 4170 may be accommodated in the staple assembly accommodation part 4101f.

In the present disclosure, an actuation motion may be performed through the structural coupling between the operation member 540 and the second jaw 2102, instead of including a separate jaw-driving pulley.

In addition, the end tool 4100 of the present embodiment can perform actuation without the pulley. For example, due to an elastic member 4109, the second jaw 4102 may maintain an open state with respect to the first jaw 4101 as a default state thereof, and as the operation member 4540 moves, a clamp 4546 is coupled to the second jaw 4102 and specifically moves into the interior of the second jaw 4102 through the guide part 4102c of the second jaw 4102, so that, as the clamp 4546 moves forward, the first jaw 4101 and the second jaw 4102 may naturally maintain a closed state.

### <Cartridge>

Hereinafter, the cartridge 4500 of the end tool 4100 of the surgical instrument of FIG. 81 will be described in more detail.

The cartridge 4500 may be disposed in the first jaw 4101, and for example, the cartridge 4500 may be disposed by being coupled to the cartridge accommodation part 4101b of the first jaw 4101. For example, the cartridge 4500 may be formed integrally with the first jaw 4101 while being connected to the retraction wire 4302 and the backward-movement prevention release wire 4303.

In an optional embodiment, the cartridge 4500 may be formed to be mountable to and dismountable from the first jaw 4101. For example, the retraction wire 4302 and the backward-movement prevention release wire 4303, which will be described later, and the operation member 4540 may be formed to be couplable to and decouplable from each other instead of being integrally formed, the cartridge 4500 may be easily decoupled from the first jaw 4101 or coupled to the first jaw 4101.

The cartridge 4500 includes a plurality of staples 4530 and a blade 4542 therein, and performs tissue stapling and cutting. Here, the cartridge 4500 may include a cover 4510, the staples 4530, withdrawal members 4535, the operation member 4540, and the reciprocating assembly 4550.

The cartridge accommodation part of the first jaw 4101 may be formed to accommodate the reciprocating assembly 4550, the operation member 4540, and the staples 4530 therein.

The cover 4510 may be formed to cover an upper portion of the cartridge accommodation part of the first jaw 4101. Staple holes through which the plurality of staples 4530 may be ejected to the outside may be formed in the cover 4510. As the staples 4530, which are accommodated inside the cartridge accommodation part of the first jaw 4101 before a stapling operation, are pushed and raised upward by the operation member 4540 during a stapling motion, and pass through the staple holes of the cover 4510 to be withdrawn to the outside of the cartridge 4500, stapling is performed.

Meanwhile, a slit may be formed in the cover 4510 along a longitudinal direction of the cover 4510. The blade 4542 of the operation member 4540 may protrude out of the cartridge 4500 through the slit. As the blade 4542 of the operation member 4540 passes along the slit, staple-completed tissue may be cut.

The plurality of staples 4530 may be disposed in the cartridge accommodation part of the first jaw 4101. As the operation member 4540 to be described later linearly moves in one direction, the plurality of staples 4530 are sequentially pushed and raised from the inside of the cartridge accommodation part of the first jaw 4101 to the outside, thereby performing suturing, that is, stapling. Here, the staples 4530 may be made of a material that may include titanium, stainless steel, or the like.

Meanwhile, the withdrawal members 4535 may be further disposed between the cartridge accommodation part of the first jaw 4101 and the staples 4530. In other words, it may be said that the staple 4530 is disposed above the withdrawal member 4535. In this case, the operation member 4540 is linearly moved in one direction to push and raise the withdrawal member 4535, and the withdrawal member 4535 may push and raise the staple 4530.

As such, the operation member 4540 may be described as pushing and raising the staples 4530 in both the case in which the operation member 4540 directly pushes and raises the staples 4530 and the case in which the operation member 4540 pushes and raises the withdrawal members 4535 and the withdrawal members 4535 pushes and raises the staples 4530 (i.e., the operation member 4540 indirectly pushes and raises the staples 4530).

The reciprocating assembly 4550 may be disposed on the inner lower side of the cartridge accommodation part of the first jaw 4101. The reciprocating assembly 4550 may include one or more reciprocating members 4551. In an embodiment, it is illustrated that one reciprocating member 4551 is provided, but a plurality of reciprocating members 4551 may be provided.

In an embodiment, the reciprocating member 4551 may be a rack. The reciprocating member 4551 may include recesses 4551b and a coupling part 4551a. In detail, the reciprocating member 4551 may be formed in the shape of an elongated bar, and a plurality of recesses 4551b having a sawtooth shape may be formed on one surface thereof. The recess 4551b may be formed to be in contact with the operation member 4540 to be described later, in particular, a ratchet member 4543 of the operation member 4540. In other words, the reciprocating member 4551 may include the plurality of recesses 4551b shaped to engage with ratchets 4543a of the ratchet member 4543.

Here, the reciprocating member 4551 is not fixedly coupled to the other components of the cartridge 4500, and may be formed to be movable relative to the other components of the cartridge 4500.

Meanwhile, in the reciprocating member 4551, the coupling part 4551a may be formed at a proximal end (see 4501 of FIG. 91) side adjacent to the pulley, and the coupling part 4551a may be fastened and coupled to the staple link assembly 4170 of the end tool.

The operation member 4540 may be disposed inside the cartridge accommodation part 4101b of the first jaw 4101. The operation member 4540 is formed to be in contact with the reciprocating member 4551, and may be formed to linearly move in one direction according to a reciprocating linear movement of the reciprocating member 4551.

The operation member 4540 may include a wedge 4541, the blade 4542, the ratchet member 4543, an operation part elastic member 4544, a body 4545, and the clamp 4546.

The body 4545 may be formed in the shape of an elongated square column, and forms a base of the operation member 4540.

The wedge 4541 is formed on at least one side of the body 4545, and may be formed to have a certain inclined surface. That is, the wedge 4541 may be formed to be inclined to a certain extent in the extension direction of the connection part 400. In other words, the wedge 4541 may be formed to have a greater height at the proximal end (see 4501 of FIG. 91) side of the cartridge 4500 than a distal end (see 4502 of FIG. 91) side of the cartridge 4500. In the drawing, it is illustrated that two wedges 4541 are formed on each side of the body 4545, but the concept of the present disclosure is not limited thereto, and the wedge 4541 may be formed in various numbers and shapes depending on the shape of the staple 4530 or the withdrawal member 4535 that is in contact with the wedge 4541.

The wedge 4541 may be formed to be in contact with the withdrawal members 4535 or the plurality of staples 4530 in turn and may serve to sequentially push and raise the staples 4530. The operation member 4540 may serve to withdraw the staples 4530 to the outside of the cartridge 4500 by sequentially pushing and raising the staples 4530 while moving toward the distal end.

The blade 4542 may be formed on one side of the wedge 4541, more specifically, on the proximal end 4501 side of the wedge 4541. An edge 4542a formed to be sharp to cut tissue is formed in one region of the blade 4542. As at least a portion of the edge 4542a is withdrawn to the outside of the first jaw 4101 and the cartridge 4500, tissue disposed between the first jaw 4101 and the second jaw 4102 may be cut. The edge 4542a of the blade 4542 may be always withdrawn to the outside of the first jaw 4101. Alternatively, the edge 4542a of the blade 4542 may normally be accommodated inside the first jaw 4101 or inside the cartridge 4500, and may be withdrawn to the outside of the first jaw 4101 only when the operation member 4540 is moved in a longitudinal direction.

The ratchet member 4543 is formed on one side of the wedge 4541, more specifically, below the wedge 4541, and may be formed to face the reciprocating member 4551 to be described later. The ratchet member 4543 may be formed in the shape of a bar and may include a plurality of ratchets 4543a on one surface. The operation member 4540 is moved only in one direction (i.e., toward the distal end) with respect to the reciprocating member 4551 by the ratchet member 4543. The ratchets 4543a of the ratchet member 4543 may be formed to be in contact with the recess 4551b of the reciprocating member 4551 described above. Meanwhile. the ratchet member 4543 may be formed to rotationally move around a rotation shaft 4547. For example, the ratchet member 4543 may be formed to rotationally move by being inserted into the rotation shaft 4547 formed in a protruding shape on the body 4545 of the operation member 4540. The rotation shaft 4547 may be disposed closer to the proximal end 4501 than the plurality of ratchets 4543a.

Meanwhile, the retraction wire 4302 may be connected to the ratchet member 4543, and by pulling the retraction wire 4302, the ratchet member 4543 may rotationally move around the rotation shaft 4547. As a specific example, the retraction wire 4302 may be connected to one region around the rotation shaft 4547. In addition, when a greater force is applied to pull the retraction wire 4302, the retraction wire 4302 may move backward, and the operation member 4540 connected to the retraction wire 4302 may also move backward (toward the proximal end).

The operation part elastic member 4544 is formed on one side of the body 4545 or the wedge 4541 and serves to apply a certain elastic force to the ratchet member 4543. As an example, the operation part elastic member 4544 may be formed such that one region is in contact with the body 4545, and another region that is different from the one region is in contact with the ratchet member 4543. The operation part elastic member 4544 may apply an elastic force in a direction in which the ratchet member 4543 comes into close contact with the reciprocating member 4551. To this end, the operation part elastic member 4544 may be formed in the form of a leaf spring, and may be provided in various forms capable of providing a certain elastic force to the ratchet member 4543, such as a coil spring, a dish spring, and the like. In addition, although not shown in the drawings, in another optional embodiment, the operation part elastic member 4544 may be integrally formed with the operation member 4540.

### <Backward-Movement Prevention Member>

The backward-movement prevention member 4548 may be formed in a manner that allows a rotational motion, such that one region thereof engages with the engaging part 4101p of the first jaw 4101, and, when at least the operation member 4540 is subjected to a force causing a backward movement, i.e., a force toward the proximal end, the backward-movement prevention member 4548 may engage with the engaging part 4101p to prevent the backward movement.

For example, the backward-movement prevention member 4548 may be formed to perform a rotational motion around a backward-movement prevention rotation shaft KJX formed on one side of the operation member 4540, and a first side 4548a (on the distal end side) and a second side 4548b (on the proximal end side) may be formed around the backward-movement prevention rotation shaft KJX. The backward-movement prevention member 4548 may perform a rotational motion around the backward-movement prevention rotation shaft KJX, and may rotate in a direction that allows the second side 4548b (on the proximal end side) to come into contact and engage with the engaging part 4101p of the first jaw 4101, or in the opposite direction to move away from the engaging part 4101p.

A backward-movement prevention elastic member 4549 may be formed on one side of the body 4545 or the wedge 4541 and may apply a certain elastic force to the first side 4548a (on the distal end side) of the backward-movement prevention member 4548. The backward-movement prevention elastic member 4549 may apply an elastic force in a direction that brings the first side 4548a (on the distal end side) closer to the ratchet member 4543. Through this, the second side 4548b, which is opposite to the first side 4548a, may be subjected to a force in a direction away from the ratchet member 4543, i.e., in a direction closer to the engaging part 4101p of the first jaw 4101.

The backward-movement prevention elastic member 4549 may effectively prevent the backward-movement prevention member 4548 from losing its functionality because the first side 4548a of the backward-movement prevention member 4548 excessively moves away from the ratchet member 4543. As a result, when a certain force or more is applied to the first side 4548a of the backward-movement prevention member 4548, causing the first side 4548a to move closer and engage with the engaging part 4101p of the first jaw 4101, the backward movement of the operation member 4540 can be stably prevented through the engagement of the backward-movement prevention member 4548 and the engaging part 4101p.

Meanwhile, the second side 4548b of the backward-movement prevention member 4548 (on the proximal end side) may be connected to the backward-movement prevention release wire 4303. The backward-movement prevention release wire 4303 may be disposed to apply a force in the same direction as the retraction wire 4302, for example, in a direction in which the operation member 4540 moved backward, i.e., toward the proximal end (see 4501 of FIG. 91).

When the backward-movement prevention release wire 4303 is connected to the second side 4548b (on the proximal end side) of the backward-movement prevention member 4548 and is pulled, the backward-movement prevention release wire 4303 provides a driving force for the backward-movement prevention member 4548 to rotate around the backward-movement prevention rotation shaft KJX. Specifically, the driving force causes the first side 4548a to rotationally move in a direction that brings the first side 4548a into close contact with the backward-movement prevention elastic member 4549, while simultaneously moving the second side 4548b in the opposite direction, away from the engaging part 4101p.

The backward-movement prevention release wire 4303 may be connected through a slot 4548h of the second side 4548b. The backward-movement prevention release wire 4303 may be fixed to a wire connection member 4303 by connecting an end portion thereof to the wire connection member 4303 through an insertion or the like. A coupling protrusion 4304b may be formed in one region, such as an end portion, of the wire connection member 4303, and the coupling protrusion 4304b may correspond to the slot 4548h of the second side 4548b. The slot 4548h of the second side 4548b may be formed larger than the coupling protrusion 4304b, and accordingly, when the backward-movement prevention release wire 4303 is pulled, the backward-movement prevention member 4548 can easily rotate and move in a direction in which the second side 4548b is quickly away from the engaging part 4101p.

Meanwhile, the wire connection member 4304 is formed to have a space adjacent to the coupling protrusion 4304b and specifically, formed in an approximately “U” shape in cross section. One region of the second side 4548b may be disposed in the space adjacent to the coupling protrusion 4304b, and for smooth movement of the second side 4548b, the space adjacent to the coupling protrusion 4304b may be formed larger than a region of the second side 4548b disposed therein.

The clamp 4546 may be formed on one side of the blade 4542 and may be formed in a shape that is approximately parallel to the body 4545 or the wedge 4541. In addition, a protrusion 4546a may be formed at one end portion of the clamp 4546, and the protrusion 4546a may be moved along the passageway. The clamp 446 may be coupled to the second jaw and, as a specific example, brought into the interior of the second jaw through the guide part of the second jaw, and as the clamp 4546 moves forward, the first jaw 4101 and the second jaw may naturally maintain a closed state.

Here, as an example, the clamp 4546 may be formed parallel to the wedge 4541 and configured to have an overall ‘U’ shape, thereby better withstanding a strong pressure applied to the wedge 4541 during the stapling motion.

Here, a first surface with a gentle slope (the surface on the distal end side) and a second surface that is vertical or nearly vertical (the surface on the proximal end side) may be formed on the ratchet 4543a of the ratchet member 4543, and since details thereof are substantially the same as those described in the ratchet member 543 in the third embodiment described above, detailed descriptions are omitted.

In addition, to engage with the ratchet 4543a of the ratchet member 4543, the recess 4551b of the reciprocating member 4551 may also include a first surface with a gentle slope and a second surface that is vertical or nearly vertical, and since details thereof are substantially the same as those described in the reciprocating member 551 of the first embodiment described above, detailed descriptions are omitted.

In a state in which the reciprocating member 4551 and the ratchet member 4543 are coupled to each other (or engaged or in close contact with each other), the reciprocating member 4551 and the ratchet member 4543 may act as a kind of ratchet, allowing movement only in one direction.

As an example, assuming that the reciprocating member 4551 is fixed, in a state in which the reciprocating member 4551 and the ratchet member 4543 are coupled to each other (or engaged or in close contact with each other), when the reciprocating member 4551 moves toward the distal end 4502, the ratchet member 4543 also moves toward the distal end 4502 by the reciprocating member 4551.

In contrast, when the reciprocating member 4551 moves toward the proximal end 4501 in a state in which the reciprocating member 4551 and the ratchet member 4543 are coupled to each other (or engaged or in close contact with each other), only the reciprocating member 4551 moves alone toward the proximal end 4501 while the ratchet member 4543 is fixed.

### <Backward-Movement Prevention Operation>

Meanwhile, in the present embodiment, the backward-movement prevention member 4548 may effectively prevent the backward movement of the operation member 4540 and, specifically, may prevent the operation member 4540 from moving together with the reciprocating member 4551 in the direction of the proximal end 4501.

FIG. 100 is a plan view illustrating the backward-movement prevention operation of the operation member of the end tool of FIG. 81.

As shown in FIG. 100B, when the reciprocating member 4551 moves in the direction of the distal end (see 4502 of FIG. 91) (in the direction of an arrow S1), the ratchet member 4543 is pressed and also moved in the direction of the arrow S1, and the operation member 4540 is also moved in the same direction, i.e., in the direction of an arrow T1. At this time, the backward-movement prevention member 4548 may rotationally move around in the direction of an arrow P1 around the backward-movement prevention rotation shaft KJX. For example, during the forward movement of the operation member 4540 (in the T1 direction), the backward-movement prevention member 4548 may rotationally move in the P1 direction, which is a direction away from the engaging part 4101p of the first jaw 4101, by the engaging part 4101p of the first jaw 4101, and through this, the second side 4548b disengages from the engaging part 4101p of the first jaw 4101, for example, a region including an end portion of the second side 4548b moves out of a groove region of the engaging part 4101p, which corresponds to the region including the end portion of the second side 4548b, as shown in FIG. 100A. In addition, the forward movement of the operation member 4540, i.e., in the S1 direction, is not affected.

As shown in FIG. 100C, when the reciprocating member 4551 moves further in the direction of the distal end 4502 (in the direction of an arrow S2), the operation member 4540 also moves further in the same direction, i.e., in the direction of an arrow T2. At this time, the backward-movement prevention member 4548 further rotates around the backward-movement prevention rotation shaft KJX in the direction of an arrow P2, and the second side 4548b is in a state of not being engaged with the engaging part 4101p of the first jaw 4101. In addition, the forward movement of the operation member 4540, i.e., in the S2 direction, is not affected. That is, the second side 4548b corresponds to the protruding region of the engaging part 4101p of the first jaw 4101, and the backward-movement prevention member 4548 may remain in the state of not being engaged with the engaging part 4101p of the first jaw 4101.

As shown in FIG. 100D, when the reciprocating member 4551 moves further in the direction of the distal end 4502 (in the direction of an arrow S3), the operation member 4540 also moves further in the same direction, i.e., in the direction of an arrow T3. At this time, the second side 4548b may be in a state of being engaged with the engaging part 4101p of the first jaw 4101 through the forward movement of the backward-movement prevention member 4548. That is, as the first side 4548a receives an elastic force from the backward-movement prevention elastic member 4549, the second side 4548b, which is a side opposite to the first side 4548a, may rotationally move toward the engaging part 4101p to enter an engaged state. In this case, when the reciprocating member 4551 moves further in the forward direction (in the T3 direction), the second side 4548b may rotationally move again in a direction away from the engaging part 4101p of the first jaw 4101, thereby disengaging from the engaging part 4101p of the first jaw 4101 without affecting the forward movement. Conversely, as shown in FIG. 100E, when the reciprocating member 4551 moves in the direction of the proximal end 4501 (in the direction of an arrow S4), the second side 4548b may remain engaged with the engaging part 4101p of the first jaw 4101. For example, the second side 4548b may be caught on the inclined surface of the engaging part 4101p, and as a specific example, the second side 4548b may be caught by the vertical or nearly vertical surface among the two inclined surfaces forming the groove and the protruding region of the engaging part 4101p.

At this time, when the reciprocating member 4551 moves backward in the S4 direction, the ratchet member 4543 of the operation member 4540 may also rotationally move (in the T4 direction), thereby releasing the ratchet coupling with the reciprocating member 4551, and in this case, the release may not be complete, but a slight connection may remain, which could undesirably cause the operation member 4540 to move backward (i.e., move toward the proximal end) along with the reciprocating member 4551. In an embodiment, the second side 4548b of the backward-movement prevention member 4548 remains engaged with the engaging part 4101p of the first jaw 4101, thereby preventing the backward movement of the operation member 4540, maintaining the stationary state, and as a result, implementing the prevention of backward movement of the operation member 4540. As shown in FIG. 100F, as the reciprocating member 4551 moves backward while the operation member 4540 remains stationary (with only the ratchet member 4543 rotating in the T5 direction), the ratchet member 4543 may re-engage with the reciprocating member 4551, and when the reciprocating member 4551 moves further backward, the state as shown in FIG. 100E is restored, thereby enabling the implementation of the backward-movement prevention operation.

### <Backward-Movement Prevention Release and Backward Movement>

Meanwhile, in the present disclosure, the retraction wire 4302 is provided to enable the backward movement of the operation member 4540, i.e., the movement of the operation member 4540 toward the proximal end 4105.

FIG. 101 is a plan view illustrating the backward movement of the operation member of the end tool of FIG. 81.

As described above, the ratchet member 4543 may be formed to be rotatable around the rotation shaft 4547, and as a specific example, the ratchet member 4543 may be inserted into the rotation shaft 4547 formed in a shape protruding from the body 4545 of the operation member 4540 and may rotationally move around the rotation shaft 4547.

The retraction wire 4302 may be connected to the ratchet member 4543, and by pulling the retraction wire 4302, the ratchet member 4543 may rotationally move around the rotation shaft 4547. As a specific example, the retraction wire 4302 may be connected to one region around the rotation shaft 4547.

When the retraction wire 4302 is pulled in the direction of an arrow JT of FIG. 101B, the ratchet member 4543 is rotated around the rotation shaft 4547 in the direction of an arrow T7 of FIG. 101B.

Here, when a certain amount or more of force is applied to the retraction wire 4302, the ratchet member 4543 is spaced away from the reciprocating member 4551 to generate a separation gap W1, and the coupling (engagement) between the ratchet member 4543 and the reciprocating member 4551 is released, so that the operation member 4540 becomes movable with respect to the reciprocating member 4551.

At this time, when the backward-movement prevention release wire 4303 is pulled in the direction of the arrow JT, similar to the retraction wire 4302, a force is applied to the second side 4548b of the backward-movement prevention member 4548, which causes the backward-movement prevention member 4548 to rotate around the backward-movement prevention rotation shaft KJX so that the second side 4548b rotationally moves in the P7 direction away from the engaging part 4101p of the first jaw 4101. As a result, the engagement between the second side 4548b and the engaging part 4101p is released, and a gap W2 is formed.

In this state, when the backward-movement prevention release wire 4303 and the retraction wire 4302 are further pulled in the direction of the arrow JT of FIG. 101C, the ratchet member 4543 is in a state of being spaced apart from the reciprocating member 4551, and the backward-movement prevention member 4548 is in a state of being spaced apart from the engaging part 4101p, causing the operation member 4540 to move entirely in the direction of the arrow T10 of FIG. 101C.

As a result, the backward movement of the operation member 4540, i.e., the movement of the operation member 4540 toward the proximal end 4105, may be enabled by the backward-movement prevention release wire 4303 and the retraction wire 4302.

As a result, during the stapling process, in which the reciprocating member 4551 of the cartridge 4500 repeatedly moves forward and backward, when the reciprocating member 4551 moves forward, the operation member 4540 moves forward together with the reciprocating member 4551, and when the reciprocating member 4551 moves backward, only the reciprocating member 4551 moves backward, and the operation member 4540 remains stationary in place without moving backward. As the operation member 4540 moves forward while repeating this process, the staple 4530 may be stapled by the wedge 4541 while the blade 4542 cuts the stapled tissue.

In addition, when the operation member 4540 needs to be moved backward again toward the proximal end, the operation member 4540 may be easily moved backward by pulling the operation member 4540 through the backward-movement prevention release wire 4303 and the retraction wire 4302.

### Fourth Embodiment of Surgical Instrument

Hereinafter, an end tool 5100 of a surgical instrument according to a fourth embodiment of the present disclosure will be described. Here, the end tool 5100 of the surgical instrument according to the fourth embodiment of the present disclosure is different from the end tool (see 2100 of FIG. 2 or the like) of the surgical instrument according to the first embodiment of the present disclosure described above in that a configuration of a second jaw 5102, a second jaw pulley 5121, a staple pulley assembly 5160, and a staple link assembly 5170 is different. Hereinafter, detailed descriptions will be provided, focusing on the configuration different from that of the first embodiment.

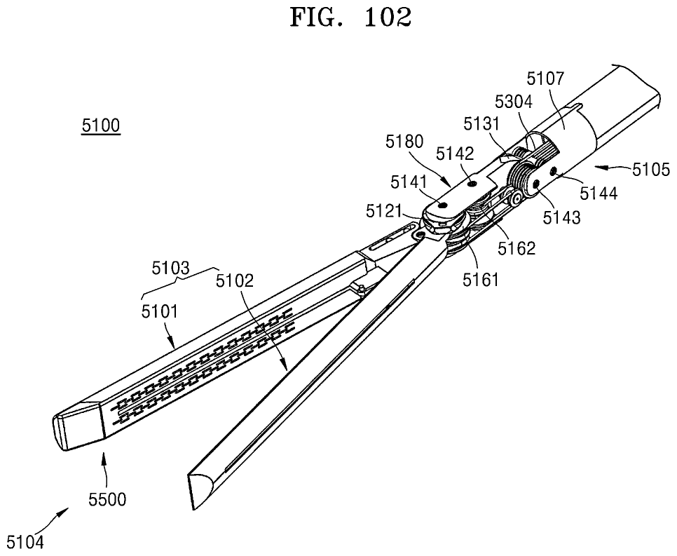

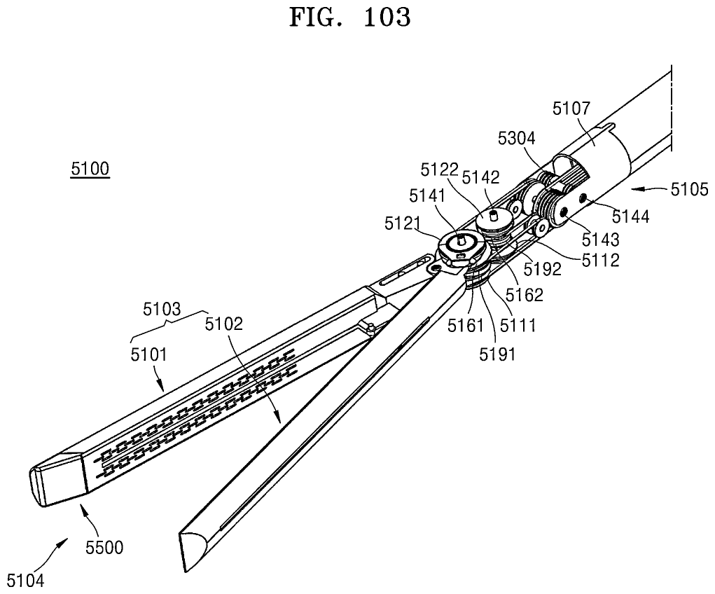

FIGS. 102 and 103 are perspective views illustrating the end tool of the surgical instrument according to the fourth embodiment of the present disclosure.

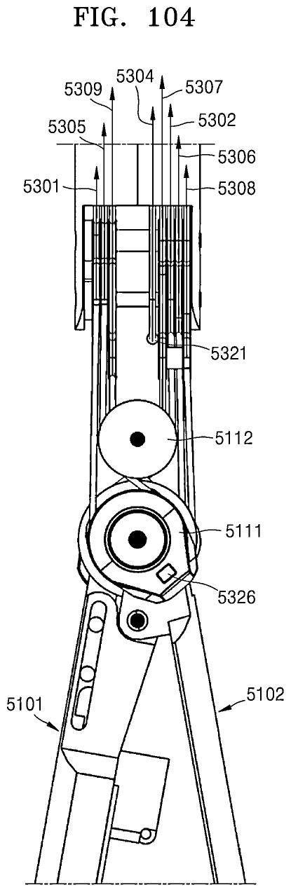

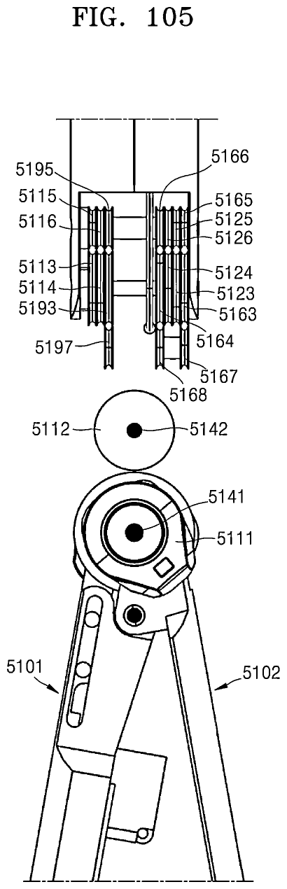

FIGS. 104 and 105 are plan views illustrating the end tool of the surgical instrument of FIG. 102.

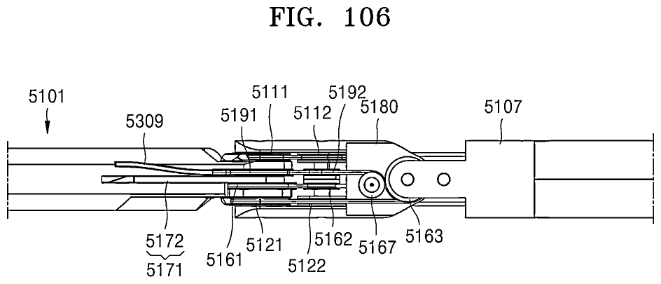

FIG. 106 is a side view illustrating the end tool of the surgical instrument of FIG. 102.

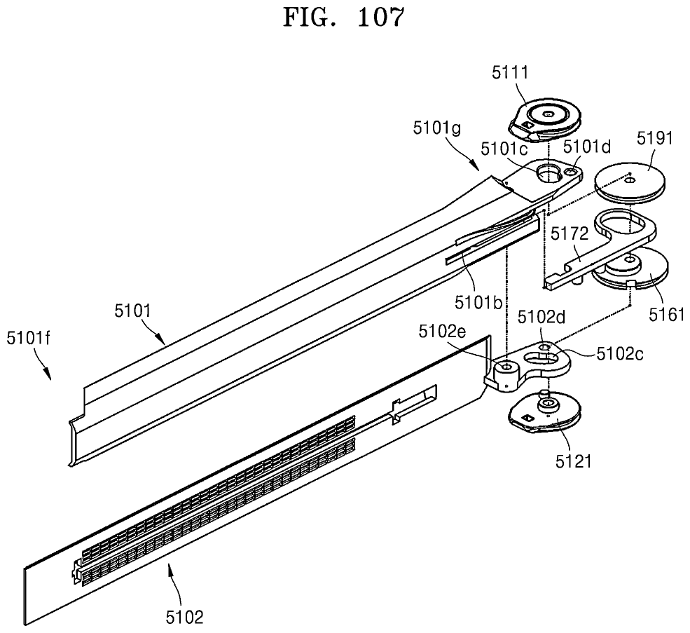

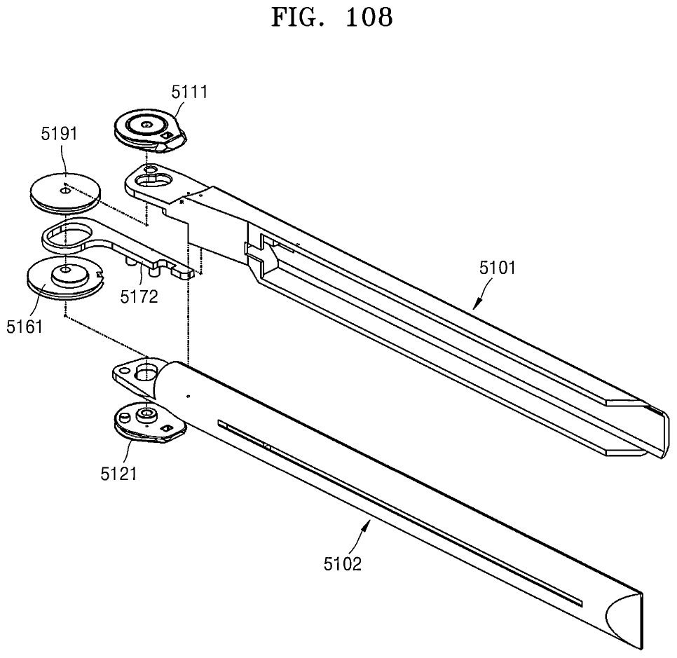

FIGS. 107 and 108 are exploded perspective views of the end tool of the surgical instrument of FIG. 102.

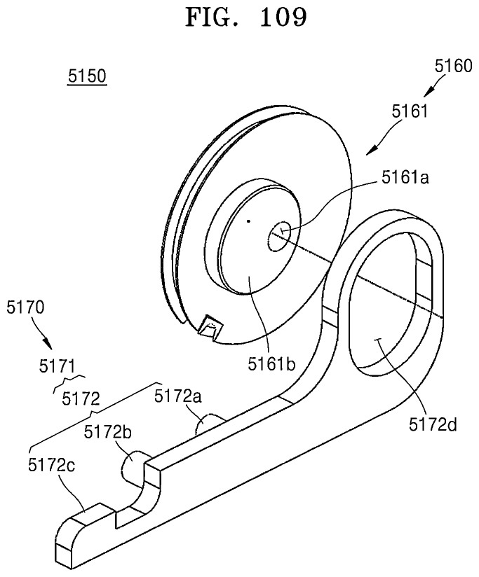

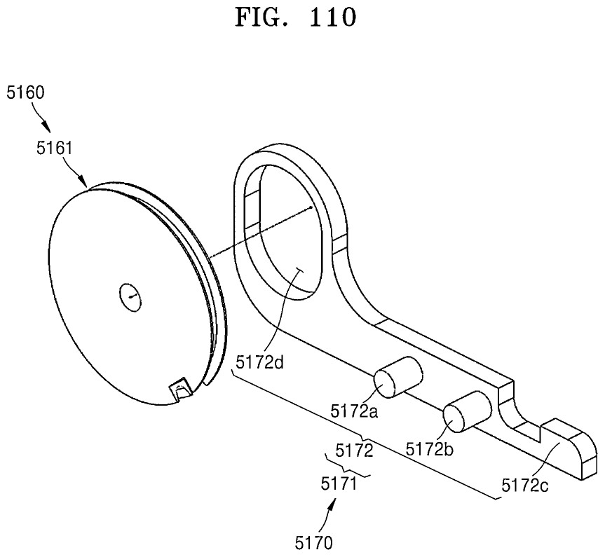

FIGS. 109 and 110 are exploded perspective views of the staple pulley assembly of the surgical instrument of FIG. 102.

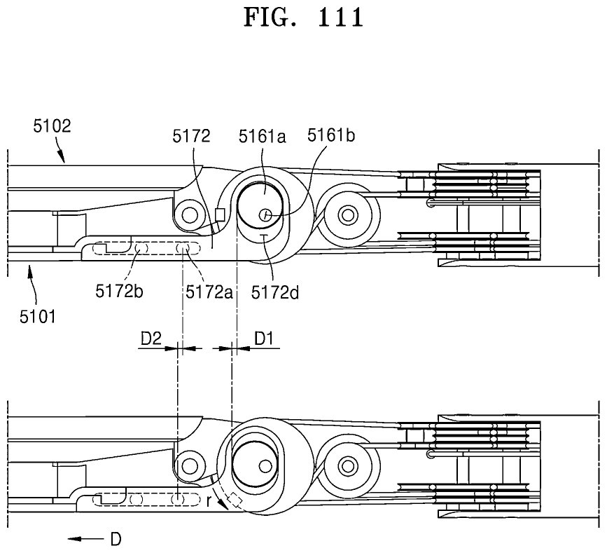

FIG. 111 is a side view illustrating operating states of a staple pulley in the end tool of FIG. 102.

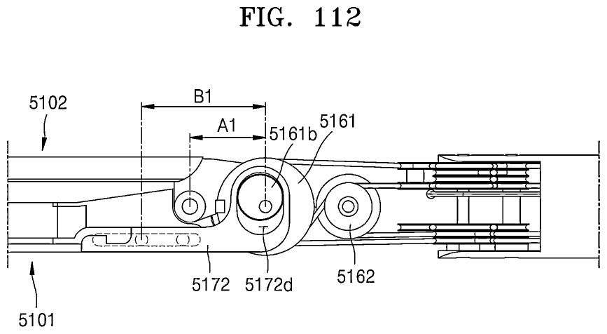

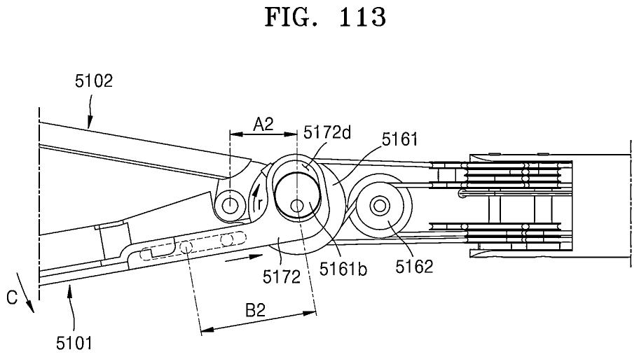

FIGS. 112 and 113 are plan views illustrating opening and closing motions of the end tool of the surgical instrument of FIG. 102.

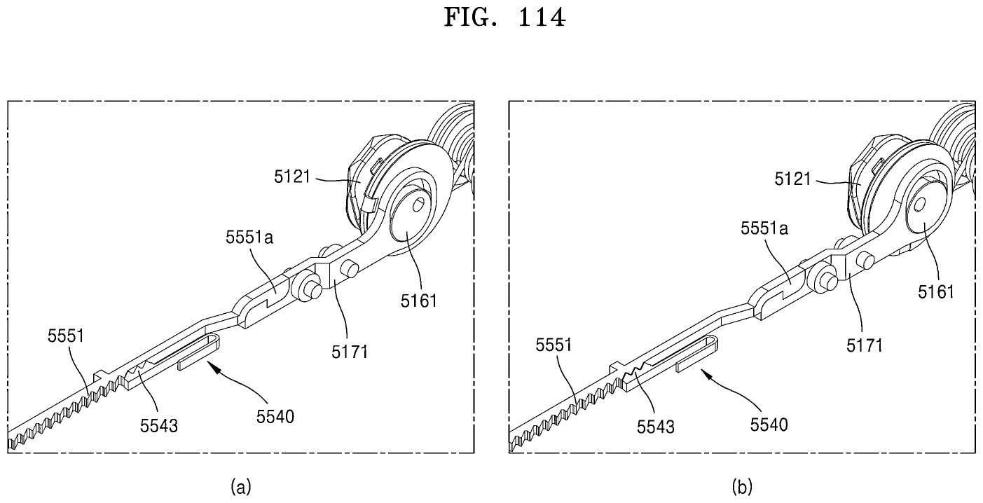

FIG. 114 is a perspective view illustrating a process in which the end tool of the surgical instrument of FIG. 102 is switched from a deactivated state to an activated state.

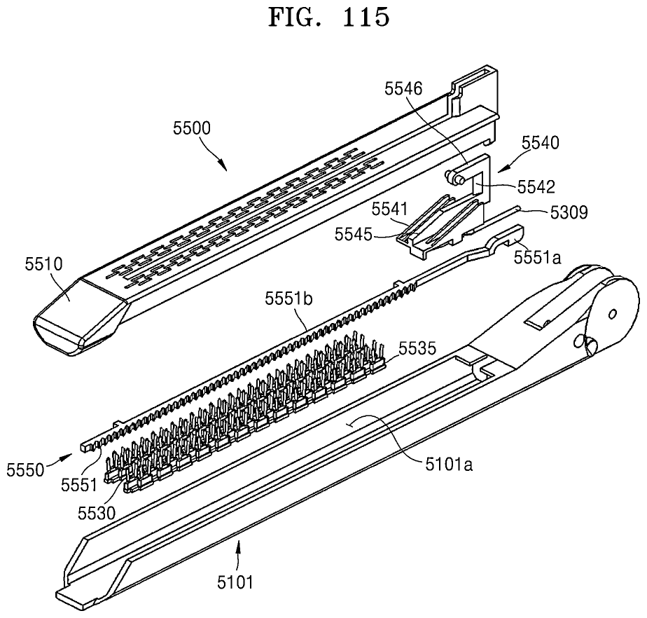

FIG. 115 is a perspective view illustrating a first jaw and a cartridge of the surgical instrument of FIG. 102.

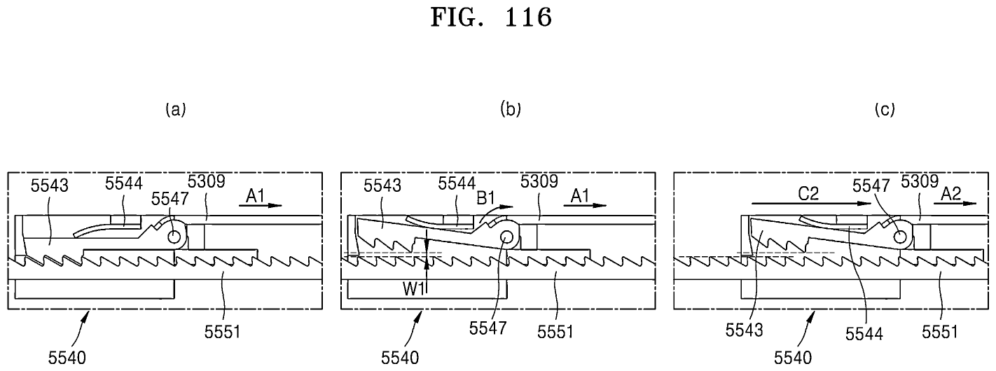

FIG. 116 is a plan view illustrating a backward movement of an operation member of the end tool of FIG. 102.

Here, FIG. 103 illustrates a state in which an end tool hub is removed. FIGS. 111 to 113 mainly describe operations of the staple pulley assembly, the staple link assembly, and a reciprocating assembly. FIG. 114 mainly describes a coupling relationship between a reciprocating member and the operation member.

Referring to FIGS. 102 to 106 and the like, a power transmission part of a surgical instrument 5100 according to an embodiment of the present disclosure may include a wire 5301, a wire 5302, a wire 5304, a wire 5305, a wire 5306, a wire 5307, a wire 5308, and a retraction wire 5309.

Here, the wire 5301 and the wire 5305 may be paired to serve as first jaw wires, and a description thereof is substantially the same as that of the wires 301 and 305 in the first embodiment described above and will therefore be omitted.

The wire 5302 and the wire 5306 may be paired to serve as second jaw wires. Here, the components encompassing the wire 5301 and the wire 5305, which are first jaw wires, and the wire 5302 and the wire 5306, which are second jaw wires, may be referred to as jaw wires.

In addition, the wire 5303 and the wire 5304 may be paired to serve as pitch wires.

In addition, the wire 5307 and the wire 5308 may be paired to serve as staple wires.

The wire 5309 may act as the retraction wire 5309, enabling the operation member 5540 to move backward toward the proximal end.

In addition, the power transmission part of the surgical instrument 5100 according to an embodiment of the present disclosure may include a coupling member (see 5321 of FIG. 104), a coupling member (see 5326 of FIG. 104), and the like that are coupled to respective end portions of the wires to respectively couple the wires to the pulleys. Here, each of the coupling members may have various shapes as necessary, such as a ball shape, a tube shape, and the like.

Here, on the end tool 5100 side, the coupling member (see 5321 of FIG. 104) may serve as a pitch wire-end tool coupling member, the coupling member (see 5326 of FIG. 104) may serve as a second jaw wire-end tool coupling member, one or more coupling members (not shown) may serve as a staple wire-end tool coupling member, and one or more coupling members (not shown) may serve a first jaw wire-end tool coupling member.

In addition, on the corresponding manipulation part (not shown) side, a first jaw wire-manipulation part coupling member and a second jaw wire-manipulation part coupling member may be disposed, and, in addition, a pitch wire-manipulation part coupling member and a staple wire-manipulation part coupling member may be further formed.

The wire 5302 and the wire 5306, which are second jaw wires, are coupled to the coupling member (see 5326 of FIG. 104), which is a second jaw wire-end tool coupling member, and the second jaw wire-manipulation part coupling member (not shown), respectively. These coupling members receive a driving force from a motor or human force, and the wires 5302 and 5306 are pulled and released by the driving force, thereby enabling the pulley 5121 of the end tool 5100 to rotate.

The wire 5307 and the wire 5308, which are staple wires, are coupled to the staple wire-end tool coupling member (not shown) and the staple wire-manipulation part coupling member (not shown), respectively. The staple wire-end tool coupling member is coupled to the staple pulley 5161, and the staple wire-manipulation part coupling member (not shown) is coupled to a pulley different from the staple pulley 5161. As a result, the staple pulley 5161 of the end tool 5100 may be rotated as the wire 5307 and the wire 5308 are pulled and released when the pulley coupled to the staple wire-manipulation part coupling member is rotated by a motor or human force.

Referring to FIGS. 102 to 114, the end tool 5100 of the fourth embodiment of the present disclosure includes a pair of jaws 5103 for performing a grip motion, that is, a first jaw 5101 and the second jaw 5102. Here, each of the first jaw 5101 and the second jaw 5102, or a component encompassing the first jaw 5101 and the second jaw 5102 may be referred to as the jaw.

Meanwhile, the end tool 5100 includes a plurality of pulleys including a pulley 5111 and a pulley 5112 that are related to a rotational motion of the first jaw 5101. The pulleys related to the rotational motion of the first jaw 5101 described in the present embodiment are substantially the same as the pulley 2111, the pulley 2112, the pulley 2113, the pulley 2114, the pulley 2115, and the pulley 2116 described with reference to FIGS. 4 to 11 and the like of the first embodiment, and thus, a detailed description thereof will be omitted herein.

Meanwhile, the end tool 5100 includes a plurality of pulleys including a pulley 5121 and a pulley 5122 that are related to a rotational motion of the second jaw 5102.

The pulley 5111 and the pulley 5121, which are end tool jaw pulleys, are formed to face each other, and are formed to be rotatable independently of each other around a rotation shaft 5141, which is an end tool jaw pulley rotation shaft. In this case, the pulley 5111 and the pulley 5121 are formed to be spaced apart from each other by a certain extent, and a staple assembly accommodation part may be formed therebetween. In addition, at least some of the staple pulley assembly 5160 and the staple link assembly 5170, which will be described below, may be disposed inside the staple assembly accommodation part.

The pulley 5112 functions as an end tool first jaw auxiliary pulley, and the pulley 5122 functions as an end tool second jaw auxiliary pulley, and these two components may be collectively referred to as an end tool jaw auxiliary pulley or simply an auxiliary pulley.

In detail, the pulley 5112 and the pulley 5122, which are end tool jaw auxiliary pulleys, may be additionally provided on one side of the pulley 5111 and one side the pulley 5121, respectively. In other words, the pulley 5112, which is an auxiliary pulley, may be disposed between the pulley 5111 and a pulley 5113 and/or a pulley 5114. In addition, the pulley 5122, which is an auxiliary pulley, may be disposed between the pulley 5121 and a pulley 5123 and/or a pulley 5124. The pulley 5112 and the pulley 5122 may be formed to be rotatable independently of each other around a rotation shaft 5142. Here, in the drawings, it is illustrated that the pulley 5112 and the pulley 5122 are formed to rotate around one rotation shaft 5142, but it is of course possible that each of the pulley 5112 and the pulley 5122 may be formed to be rotatable around a separate shaft. In addition, the configuration and function of the end tool auxiliary pulley may be modified and applied within a range substantially the same as or similar to those described in the end tool of the first embodiment described above, and thus, further detailed descriptions thereof will be omitted.

Components associated with the rotation of the pulley 5121, which is a pulley related to the second jaw 5102, will be described.

The pulley 5123 and the pulley 5124 function as end tool second jaw pitch main pulleys. That is, the pulley 5123 and the pulley 5124 function as main rotation pulleys for a pitch motion of the second jaw 5102. Here, the wire 5306, which is a second jaw wire, is wound around the pulley 5123, and the wire 5302, which is a second jaw wire, is wound around the pulley 5124.

A pulley 5125 and a pulley 5126 function as end tool second jaw pitch sub-pulleys. That is, the pulley 5125 and the pulley 5126 function as sub-rotation pulleys for a pitch motion of the second jaw 5102. Here, the wire 5306, which is a second jaw wire, is wound around the pulley 5125, and the wire 5302, which is a second jaw wire, is wound around the pulley 5126.

On one side of the pulley 5121, the pulley 5123 and the pulley 5124 are disposed to face each other. Here, the pulley 5123 and the pulley 5124 are formed to be rotatable independently of each other around a rotation shaft 5143 that is an end tool pitch rotation shaft. In addition, the pulley 5125 and the pulley 5126 are disposed on one side of the pulley 5123 and one side of the pulley 5124, respectively, to face each other. Here, the pulley 5125 and the pulley 5126 are formed to be rotatable independently of each other around a rotation shaft 5144 that is an end tool pitch auxiliary rotation shaft. Here, in the drawings, it is illustrated that all of the pulley 5123, the pulley 5125, the pulley 5124, and the pulley 5126 are formed to be rotatable around the Y-axis direction, but the concept of the present disclosure is not limited thereto, and the rotation shafts of the respective pulleys may be formed in various directions according to configurations thereof.

The wire 5306, which is a second jaw wire, is sequentially wound to make contact with at least portions of the pulley 5125, the pulley 5123, and the pulley 5121. In addition, the wire 5302 connected to the wire 5306 by the coupling member 5326 is sequentially wound to make contact with at least portions of the pulley 5121, the pulley 5122, the pulley 5124, and the pulley 5126.

In other words, the wire 5306 and the wire 5302, which are second jaw wires, are sequentially wound to make contact with at least portions of the pulley 5125, the pulley 5123, the pulley 5121, the pulley 5122, the pulley 5124, and the pulley 5126, and are formed to move along the above pulleys while rotating the above pulleys.

Accordingly, when the wire 5306 is pulled in the direction of an arrow 5306 of FIG. 104, the coupling member to which the wire 5306 is coupled and the pulley 5121 coupled thereto rotate in one direction, and in contrast, when the wire 302 is pulled in the direction of an arrow 302 of FIG. 104, the coupling member to which the wire 5302 is coupled and the pulley 5121 coupled thereto rotate in a direction opposite to the one direction.

The retraction wire 5309 may be sequentially wound to make contact with at least portions of a pulley 5191, a pulley 5192, and the like.

The pulley 5191, which is a pulley for the retraction wire, may be disposed adjacent to or in a space between the pulley 5111 and the pulley 5121.

The pulley 5192, which is an auxiliary pulley for the retraction wire, may be disposed on one side of the pulley 5191 to be closer the proximal end side than the pulley 5191, and may be disposed adjacent to or in the space between the pulley 5112 and the pulley 5122.

When the retraction wire 5309 is pulled toward an arrow 5309 of FIG. 104, the retraction wire 5309 may provide a driving force to cause the operation member 5540 to move backward.

Hereinafter, a pitch motion of the present disclosure will be described in more detail.

Meanwhile, when the wire 5301 is pulled toward an arrow 5301 of FIG. 104, and simultaneously, the wire 5305 is pulled toward an arrow 5305 of FIG. 104 (that is, when both strands of the first jaw wire are pulled), since the wires 5301 and 5305 are wound around lower portions of the pulley 5113 and the pulley 5114 rotatable around the rotation shaft 5143, which is an end tool pitch rotation shaft, the pulley 5111 to which the wires 5301 and 5305 are fixedly coupled and an end tool hub 5180 to which the pulley 5111 is coupled are rotated as a whole in the counterclockwise direction around the rotation shaft 5143, so that the end tool 5100 is rotated downward to perform a pitch motion. At this time, since the second jaw 5102 and the wires 5302 and 5306 fixedly coupled thereto are wound around upper portions of the pulley 5123 and the pulley 5124 rotatable around the rotation shaft 5143, the wires 5302 and 5306 are unwound in opposite directions of the arrows 5302 and 5306, respectively.

In contrast, when the wire 5302 is pulled toward an arrow 5302 of FIG. 104, and simultaneously, the wire 5306 is pulled toward the arrow 5306 of FIG. 104, since the wires 5302 and 5306 are wound around the upper portions of the pulley 5123 and the pulley 5124 rotatable around the rotation shaft 5143, which is an end tool pitch rotation shaft, the pulley 5121 to which the wires 5302 and 5306 are fixedly coupled and the end tool hub 5180 to which the pulley 5121 is coupled are rotated as a whole in the clockwise direction around the rotation shaft 5143, so that the end tool 5100 is rotated upward to perform a pitch motion. At this time, since the first jaw 5101 and the wires 5301 and 5305 fixedly coupled thereto are wound around lower portions of the pulley 5113 and the pulley 5114 rotatable around the rotation shaft 5143, the wires 5302 and 5306 are moved in opposite directions of the wires 5301 and 5305, respectively.

Further, the end tool 5100 of the fourth embodiment of the present disclosure may include the rotation shaft 5141, the rotation shaft 5142, the rotation shaft 5143, and the rotation shaft 5144. Here, the rotation shaft 5141 and the rotation shaft 5142 may be inserted through an end tool hub 5180, and the rotation shaft 5143 and the rotation shaft 5144 may be inserted through a pitch hub 5107. The rotation shaft 5141, the rotation shaft 5142, the rotation shaft 5143, and the rotation shaft 5144 may be arranged sequentially from a distal end 5104 toward a proximal end 5105 of the end tool 5100.

In addition, the end tool 5100 of the fourth embodiment of the present disclosure may include the end tool hub 5180 and the pitch hub 5107.

The rotation shaft 5141 and the rotation shaft 5142 may be inserted through the end tool hub 5180, and the pulley 5111 and the pulley 5121 axially coupled to the rotation shaft 5141 and at least some of the first jaw 5101 and the second jaw 5102 coupled to the pulley 5111 and the pulley 5121 may be accommodated inside the end tool hub 5180.

The rotation shaft 5143 and the rotation shaft 5144 may be inserted through the pitch hub 5107, and the pitch hub 5107 may be axially coupled to the end tool hub 5180 by the rotation shaft 5143. Accordingly, the end tool hub 5180 may be formed to be pitch-rotatable around the rotation shaft 5143 with respect to the pitch hub 5107.

Meanwhile, the end tool 5100 of the fourth embodiment of the present disclosure may further include components, such as a staple drive assembly (see 5150 of FIGS. 109 and 110) including the staple pulley assembly 5160 and the staple link assembly 5170, to perform stapling and cutting motions.

The staple pulley assembly 5160 may be formed between the pulley 5111 and the pulley 5121 to be adjacent to the pulley 5111 and the pulley 5121. In an embodiment, it is assumed that the staple pulley assembly 5160 includes one staple pulley 5161.

In the fourth embodiment of the present disclosure, by disposing the staple pulley assembly 5160 between the pulley 5111, which is a first jaw pulley, and the pulley 5121, which is a second jaw pulley, the end tool 5100 is allowed to perform pitch and yaw motions as well as stapling and cutting motions using a cartridge 5500.

Hereinafter, the staple pulley assembly 5160, the staple link assembly 5170, and a reciprocating assembly 5550 of the end tool 5100 of the surgical instrument according to the fourth embodiment of the present disclosure will be described in more detail.

In the end tool 5100 of the surgical instrument according to the fourth embodiment of the present disclosure, the staple pulley assembly 5160 and the staple link assembly 5170 form a cam-slot structure. In addition, such a structure provides the effect of amplifying a force for moving the reciprocating assembly 5550 forward. Furthermore, a deactivated state, in which the staple link assembly 5170 and the reciprocating assembly 5550 are separated from each other, can be switched to an activated state in which the staple link assembly 5170 and the reciprocating assembly 5550 are coupled to each other.

Referring to FIGS. 102 to 114 and the like, the staple pulley assembly 5160 may include one or more staple pulleys 5161.

A shaft pass-through part 5161a may be formed in the staple pulley 5161. The shaft pass-through part 5161a may be formed in the form of a hole, and the rotation shaft 5141, which is an end tool jaw pulley rotation shaft, may be inserted through the shaft pass-through part 5161a.

Further, a protruding member 5161b may be formed on the staple pulley 5161. A link member 5171 of the staple link assembly 5170 may be coupled to the protruding member 5161b. Here, the center of the protruding member 5161b does not coincide with the center of the staple pulley 5161, and the protruding member 5161b may be formed to be eccentric to a certain extent with respect to the staple pulley 5161. The protruding member 5161b may be fitted into a slot 5172d of the link member 5171 to be described later. Meanwhile, the end tool 5100 of the fourth embodiment of the present disclosure may further include the staple link assembly 5170 connected to the staple pulley assembly 5160, and the staple link assembly 5170 may include the link member 5171. Here, the staple link assembly 5170 may serve to connect the staple pulley assembly 5160 to the reciprocating assembly 5550 of the cartridge 5500, which will be described later.

In an embodiment, the staple link assembly 5170 includes only one link. That is, by coupling the staple pulley assembly 5160 to the staple link assembly 5170 using a cam-slot structure, a rotational motion of the staple pulley assembly 5160 can be converted into a linear motion of the staple link assembly 5170 even when the staple link assembly 5170 includes only one link.

In detail, the link member 5171 may be formed as a single link, which is a first link 5172. In other words, the first link 5172 may be formed as a single member.

The first link 5172 is formed in the shape of an elongated bar, and may include a first protrusion 5172a, a second protrusion 5172b, a coupling part 5172c, and the slot 5172d.

The first protrusion 5172a and the second protrusion 5172b may be formed in one region of a central portion of the first link 5172. The first protrusion 5172a and the second protrusion 5172b may be fitted into a guide groove 5101b of the first jaw 5101.

As described above, as the first protrusion 5172a and the second protrusion 5172b are moved along the guide groove 5101b in a state in which the first protrusion 5172a and the second protrusion 5172b of the first link 5172 formed in a protruding shape are fitted into the groove-shaped guide groove 5101b, the link member 5171 is moved with respect to the first jaw 5101 (and the cartridge 5500 therein). This will be described in more detail later.

Meanwhile, the coupling part 5172c may be formed at one end portion of the first link 5172. The coupling part 5172c may be coupled to a coupling part 5551a of a reciprocating member 5551 of the cartridge 5500, which will be described later.

Meanwhile, the slot 5172d may be formed at one end portion of the first link 5172. Here, the slot 5172d may be formed in the shape of an elongated hole, into which the protruding member 5161b may be fitted. In detail, the slot 5172d may be formed to have a certain curvature, and may be formed in an approximately elliptical shape. Here, the slot 5172d may be formed to be larger than the protruding member 5161b by a certain extent. Accordingly, in a state in which the protruding member 5161b of the staple pulley 5161 is fitted into the slot 5172d of the first link 5172, the protruding member 5161b is moved to a certain extent in the slot 5172d.

As described above, the protruding member 5161b may be formed to be eccentric with respect to the staple pulley 5161 by a certain extent. Accordingly, when the staple pulley 5161 is rotated, the protruding member 5161b, while in contact with the slot 5172d, may push the slot 5172d to move the first link 5172. That is, when the staple pulley 5161 is rotated, the protruding member 5161b is moved in the slot 5172d while coming into contact with the slot 5172d, which causes the first link 5172 to be linearly moved along the guide groove 5101b of the first jaw 5101.

### (Displacement of Staple Link Assembly According to Rotation of Staple Pulley)

Hereinafter, the displacement of the staple link assembly 5170 according to the rotation of the staple pulley 5161 will be described.

Referring to FIGS. 109 to 111 and the like, in the fourth embodiment of the present disclosure, the staple pulley 5161 and the staple link assembly 5170 are coupled to each other in a cam-slot configuration. That is, the protruding member 5161b formed on the staple pulley 5161 is coupled to the slot 5172d formed in the form of a slot in the staple link assembly 5170. Accordingly, when the staple pulley 5161 is rotated in the direction of an arrow r, the displacement of the protruding member 5161b of the staple pulley 5161 in the X-axis direction becomes D1. In addition, the displacement of the staple link assembly 5170 in the X-axis direction becomes D2.

Meanwhile, as compared to the case in which the staple pulley and the staple link assembly are axially coupled, when the staple pulley and the staple link assembly are cam-slot coupled as in the present embodiment, the displacement of the staple link assembly in the X-axis direction will be reduced even when the staple pulley is rotated by the same amount.

Meanwhile, since work is the product of force and displacement, assuming that the work for rotating the staple pulley is the same, the displacement and the force are inversely proportional to each other. Accordingly, when the displacement is reduced, the force is increased in inverse proportion to the displacement.

As a result, in the fourth embodiment of the present disclosure, the staple pulley 5161 and the staple link assembly 5170 are coupled to each other in a cam-slot configuration, and the displacement of the staple link assembly 5170 in the X-axis direction caused by the rotation of the staple pulley 5161 is relatively reduced compared to other coupling structures, and thus the force received by the staple link assembly 5170 in the X-axis direction is relatively increased compared to other coupling structures.

According to the fourth embodiment of the present disclosure described above, a force for moving the staple link assembly 5170 and the reciprocating assembly 550 connected thereto forward is amplified, thereby achieving the effect of performing a stapling motion more robustly.

### (Activated State and Deactivated State)

Hereinafter, an activated state and a deactivated state in the present embodiment will be described.

In the fourth embodiment of the present disclosure, there are an deactivated state in which the reciprocating assembly 5550 and the operation member 5540 are not coupled to and separated from each other, and an activated state in which the reciprocating assembly 5550 and the operation member 5540 are coupled. In addition, the deactivated state may be maintained in a jaw open state, and the deactivated state may be switched to the activated state in a jaw close state. In addition, after switching to the activated state, as the reciprocating assembly is repeatedly moved forward and backward according to an alternating rotational motion of the staple pulley, the operation member is moved forward.

This distinction between the activated and deactivated states may be made possible by the cam-slot structure described above. Hereinafter, this will be described in more detail.

Referring to FIGS. 112 to 114 and the like, in a state in which the jaw 5103 is closed as shown in FIG. 112, when the first jaw 5101 is rotated in the direction of an arrow C of FIG. 113 and the second jaw 5102 is rotated in the opposite direction of the arrow C, the jaw 5103 is in an opened state. Here, it is assumed that the position of a jaw rotation shaft in the X-axis direction is fixed, while the other components move.

When the jaw 5103 is in the open state, the pulley 5121, which is a second jaw pulley, and the staple pulley 5161 are rotated in the same direction.

When the pulley 5121, which is a second jaw pulley, is rotated in the direction of the arrow r of FIG. 113, the pulley 5121, the staple pulley 5161, the staple link assembly 5170, and the reciprocating assembly 5550 are moved together toward a distal end 5101f (i.e., are moved forward) in a state in which the jaw rotation shaft is fixed. Accordingly, A1 of FIG. 112, which is a distance between the rotation shaft 5141 and the jaw rotation shaft in a state in which the jaw 5103 is closed, is greater than A2 of FIG. 112, which is a distance between the rotation shaft 5141 and the jaw rotation shaft in a state in which the jaw 5103 is opened (i.e., A1>A2).

At the same time, when the staple pulley 5161 is rotated in the direction of the arrow r of FIG. 113, the staple link assembly 5170 connected to the staple pulley 5161 and the reciprocating assembly 5550 connected the staple link assembly 5170 are moved as a whole (i.e., are moved backward) toward a proximal end 5101g. Accordingly, B1 of FIG. 112, which is a distance between the rotation shaft 5141 and the staple link assembly 5170 (the second protrusion 5172b thereof) in a state in which the jaw 5103 is closed, is greater than B2 of FIG. 113, which is a distance between the rotation shaft 5141 and the staple link assembly 5170 (the second protrusion 5172b thereof) in a state in which the jaw 5103 is opened (i.e., B1>B2).

However, here, due to the cam-slot structure as described above, the displacement of the staple link assembly 5170 in the x-axis direction when the staple pulley 5161 is rotated is relatively small due to the above-described cam-slot structure.

That is, in the fourth embodiment of the present disclosure, a relationship of (A1−A2)>(B1−B2) is established.

In other words, when the jaw 5103 is opened, the movement amount (A1−A2) of the pulley 5121, the staple pulley 5161, the staple link assembly 5170, and the reciprocating assembly 5550 toward the distal end is greater than the movement amount (B1−B2) of the staple link assembly 5170 and the reciprocating assembly 5550 connected thereto toward the proximal end.

As a result, when the two movement components of the reciprocating assembly 5550 described above are synthesized, the reciprocating assembly 5550 is moved toward the distal end 5101f when the jaw 5103 is opened. That is, the reciprocating assembly 5550 is moved forward.

That is, in the present embodiment, the amount of forward movement of the reciprocating assembly caused by the rotation of the jaw pulley is greater than the amount of backward movement of the reciprocating assembly caused by the rotation of the staple pulley, thereby causing the reciprocating member to move forward when the jaws are open.

In addition, when the reciprocating assembly 5550 is moved forward as described above, the reciprocating assembly 5550 and the operation member 5540 are further spaced apart from each other, and thus, when the jaw is opened, the deactivated state in which the reciprocating assembly 5550 and the operation member 5540 are not coupled is maintained.

In contrast, in a state in which the jaw 5103 is opened as shown in FIG. 113, the first jaw 5101 is rotated in the opposite direction of the arrow C of FIG. 113, and when the second jaw 5102 is rotated in the direction of the arrow C, the jaw 5103 is in a closed state as shown in FIG. 112.

When the jaw 5103 is closed as described above, the reciprocating assembly 5550 is moved toward the proximal end 5101g. That is, the reciprocating assembly 5550 is moved backward.

In the closed state as shown in FIG. 112, a recess 5550b of the reciprocating assembly 5550 and a ratchet 5543a of a ratchet member 5543 of the operation member 5540 are in contact with each other but are not coupled to each other.

Accordingly, the deactivated state in which the reciprocating assembly 5550 and the operation member 5540 are not coupled to and separated from each other is maintained between the state of FIG. 113, in which the jaw 5103 is fully opened, and the state of FIG. 112, in which the jaw 5103 is closed. That is, in this state, even when the staple pulley 5161 is rotated, the operation member 5540 is not moved, and thus stapling and cutting motions are not performed.

Next, for stapling and cutting motions, the staple pulley 5161 is rotated (in the direction of the arrow C of FIG. 113) as shown in FIG. 114B in a state in which the jaw 5103 is closed as shown in FIG. 114A.

When the staple pulley 5161 is rotated, the staple link assembly 5170 connected to the staple pulley 5161 and the reciprocating assembly 5550 connected to the staple link assembly 5170 are moved as a whole toward the proximal end 5101g (i.e., are moved backward). In addition, as shown in FIG. 114B, when the reciprocating assembly 5550 is moved toward the proximal end 5101g to be coupled to the ratchet member 5543 of the operation member 5540, the deactivated state is switched to the activated state.

After switching to the activated state as described above, as the staple pulley 5161 performs an alternating rotational motion, the reciprocating assembly 5550 is repeatedly moved forward and backward, thereby performing stapling and cutting motions while the operation member 5540 is moved forward.

### (First and Second Jaws and Actuation Motion)

Hereinafter, a coupling structure of the first jaw 5101 and the second jaw 5102 of the end tool 5100 of the surgical instrument of FIG. 102 will be described in more detail.

The first jaw 5101 includes the cartridge accommodation part 5101a, the guide groove 5101b, the movable coupling hole 5101c, the jaw pulley coupling hole 5101d, and the shaft pass-through part.

The first jaw 5101 is formed entirely in the shape of an elongated bar, the cartridge 5500 is accommodated in the distal end 5101f side, and the pulley 5111 is coupled to the proximal end 5101g, so that the first jaw 5101 is formed to be rotatable around the rotation shaft 5141. In other words, the first jaw 5101 may be formed entirely in the form of a hollow box with one surface (upper surface) thereof removed, such that the cartridge accommodation part 5101a capable of accommodating the cartridge 5500 may be formed inside the first jaw 5101. That is, the first jaw 5101 may be formed in a substantially “U” shape in cross section.

In the first jaw 5101, the guide groove 5101b configured to guide the movement of the staple link assembly 5170 may be formed on one side of the cartridge accommodation part 5101a, e.g., on the proximal end 5101g side. The guide groove 5101b may be formed in the shape of a groove formed along a moving path of the staple link assembly 5170.

Meanwhile, the movable coupling hole 5101c, the jaw pulley coupling hole 5101d, and the shaft pass-through part may be formed on the proximal end side of the first jaw 5101.

Here, the movable coupling hole 5101c may be formed to have a certain curvature, and may be formed in an approximately elliptical shape. A shaft coupling part of the pulley 5111 may be fitted into the movable coupling hole 5101c. Here, a short radius of the movable coupling hole 5101c may be formed to be substantially the same as or slightly greater than a radius of the shaft coupling part. Meanwhile, a long radius of the movable coupling hole 5101c may be formed to be larger than the radius of the shaft coupling part. Thus, in a state in which the shaft coupling part of the pulley 5111 is fitted into the movable coupling hole 5101c of the first jaw 5101, the shaft coupling part is movable to a certain extent in the movable coupling hole 5101c.

Meanwhile, the jaw pulley coupling hole 5101d is formed in the form of a cylindrical hole, and a jaw coupling part of the pulley 5111 may be fitted into the jaw pulley coupling hole 5101d. Here, a radius of the jaw pulley coupling hole 5101d may be formed to be substantially the same as or slightly greater than a radius of the jaw coupling part of the pulley 5111. Thus, the jaw coupling part of the pulley 5111 may be formed to be rotatably coupled to the jaw pulley coupling hole 5101d of the first jaw 5101.

The shaft pass-through part may be formed at the distal end 5101f side of the first jaw 5101 relative to the movable coupling hole 5101c and the jaw pulley coupling hole 5101d, and may correspond to the jaw rotation shaft.

The second jaw 5102 includes a movable coupling hole 5102c, a jaw pulley coupling hole 5102d, and a shaft pass-through part 5102e.

The second jaw 5102 is formed in an overall elongated bar shape, and an anvil may be formed at a distal end 5102f side. The pulley 5121 is coupled to a proximal end 5102g, and may be formed to be rotatable around the rotation shaft 5141.

In detail, the anvil is formed on one surface of the second jaw 5102 in a flat plane shape, and shapes corresponding to the shapes of the staples 5530 to be described below may be formed on the one surface. The above-described anvil may serve as a support for supporting the staple 5530 on the opposite side of the operation member 5540 when the operation member 5540 pushes and raises the staple 5530 during a stapling motion, so that the staple 5530 is bent. In addition, a groove-shaped passageway may be formed on the surface of the second jaw 5102, on which the anvil is formed, to guide the movement of the operation member 5540. For example, the groove-shaped passageway may be formed such that one region of the clamp 5546 of the operation member 5540 corresponds to an inner side of the second jaw 5102.

Meanwhile, the movable coupling hole 5102c, the jaw pulley coupling hole 5102d, and the shaft pass-through part 5102e may be formed on the proximal end side of the second jaw 5102.

Here, the movable coupling hole 5102c may be formed to have a certain curvature, and may be formed in an approximately elliptical shape. A shaft coupling part of the pulley 5121 may be fitted into the movable coupling hole 5102c. Here, a short radius of the movable coupling hole 5102c may be formed to be substantially the same as or slightly greater than a radius of the shaft coupling part of the pulley 5121. Meanwhile, a long radius of the movable coupling hole 5102c may be formed to be larger than the radius of the shaft coupling part of the pulley 5121. Thus, in a state in which the shaft coupling part of the pulley 5121 is fitted into the movable coupling hole 5102c of the second jaw 5102, the shaft coupling part of the pulley 5121 is movable to a certain extent in the movable coupling hole 5102c.

Meanwhile, the jaw pulley coupling hole 5102d is formed in the form of a cylindrical hole, and a jaw coupling part of the pulley 5121 may be fitted into the jaw pulley coupling hole 5102d. Here, a radius of the jaw pulley coupling hole 5102d may be formed to be substantially the same as or slightly greater than a radius of the jaw coupling part of the pulley 5121. Thus, the jaw coupling part of the pulley 5121 may be formed to be rotatably coupled to the jaw pulley coupling hole 5102d of the second jaw 5102.

Meanwhile, the shaft pass-through part 5102e may be formed at the distal end 5102g side of the second jaw 5102 relative to the movable coupling hole 5102c and the jaw pulley coupling hole 5102d. The shaft pass-through part 5102e is formed in the shape of a hole and may correspond to the jaw rotation shaft.

### (Cartridge)

The cartridge 5500 may be disposed in the first jaw 5101, and for example, the cartridge 5500 may be disposed by being coupled to the cartridge accommodation part 5101a of the first jaw 5101. For example, the cartridge 5500 may be integrally formed with the first jaw 5101 while the operation member 5540 is connected to the retraction wire 5309.

In an optional embodiment, the cartridge 5500 may be formed to be mountable to and dismountable from the first jaw 5101. For example, the retraction wire 5309 to be described later and the operation member 5540 may be formed to be couplable to and decouplable from each other, rather than being integrally connected to each other, thereby facilitating the decoupling or coupling of the cartridge 5500 from and to the first jaw 5101.

The cartridge 5500 includes a plurality of staples 5530 and a blade 5542 therein, and performs tissue stapling and cutting. Here, the cartridge 5500 may include a cover 5510, the staples 5530, withdrawal members 5535, the operation member 5540, and the reciprocating assembly 5550.

The cartridge accommodation part of the first jaw 5101 may be formed to accommodate the reciprocating assembly 5550, the operation member 5540, and the staples 5530 therein.

In an embodiment, the reciprocating member 5551 may be a rack. The reciprocating member 5551 may include recesses 5551b and the coupling part 5551a.

The operation member 5540 may be disposed inside the cartridge accommodation part 5101a of the first jaw 5101. The operation member 5540 is formed to be in contact with the reciprocating member 5551, and may be formed to linearly move in one direction according to the reciprocating linear movement of the reciprocating member 5551.

The operation member 5540 may include a wedge 5541, the blade 5542, the ratchet member 5543, an operation part elastic member 5544, a body 5545, and the clamp 5546.

The configuration of the cartridge 5500 may be modified and applied within a range substantially the same as or similar to that of the cartridge 500 of the first embodiment described above, and thus, a detailed description thereof will be omitted.

Meanwhile, the retraction wire 5309 may be connected to the ratchet member 5543, and by pulling the retraction wire 5309, the ratchet member 5543 may rotationally move around a rotation shaft 5547. As a specific example, the retraction wire 5309 may be connected to one region around the rotation shaft 5547. In addition, when a greater force is applied to pull the retraction wire 5309, the retraction wire 5309 may move backward, and the operation member 5540 connected to the retraction wire 5309 may also move backward (toward the proximal end).

The operation part elastic member 5544 is formed on one side of the body 5545 or the wedge 5541 and serves to apply a certain elastic force to the ratchet member 5543. As an example, the operation part elastic member 5544 may be formed such that one region is in contact with the body 5545, and another region that is different from the one region is in contact with the ratchet member 5543. The operation part elastic member 5544 may apply an elastic force in a direction in which the ratchet member 5543 comes into close contact with the reciprocating member 5551. To this end, the operation part elastic member 5544 may be formed in the form of a leaf spring, and may be provided in various forms capable of providing a certain elastic force to the ratchet member 5543, such as a coil spring, a dish spring, and the like. In addition, although not shown in the drawings, in another optional embodiment, the operation part elastic member 5544 may be integrally formed with the operation member 5540.

### <Backward Movement>

FIG. 116 is a plan view illustrating the backward movement of the operation member of the end tool of FIG. 112.

The ratchet member 5543 of the operation member 5540 may be formed to be rotatable around the rotation shaft 5547, and as a specific example, the ratchet member 5543 may be inserted into the rotation shaft 5547 formed in a shape protruding from the body 5545 of the operation member 5540 and may rotationally move around the rotation shaft 5547.

The retraction wire 5309 may be connected to the ratchet member 5543, and by pulling the retraction wire 5309, the ratchet member 5543 may rotationally move around the rotation shaft 5547. As a specific example, the retraction wire 5309 may be coupled to one region around the rotation shaft 5547.

When the retraction wire 5309 is pulled in the direction of an arrow A1 of FIG. 116B, the ratchet member 5543 rotates around the rotation shaft 5547 in the direction of an arrow B1 of FIG. 116B.

Here, when a certain amount or more of force is applied to the retraction wire 5309, the ratchet member 5543 is spaced away from the reciprocating member 5551 to generate a separation gap W1, and the coupling (engagement) between the ratchet member 5543 and the reciprocating member 5551 is released, so that the operation member 5540 becomes movable with respect to the reciprocating member 5551.

In this state, when the retraction wire 5309 is pulled further in the direction of an arrow A2 of FIG. 116C, the operation member 5540 as a whole is moved in the direction of an arrow C2 of FIG. 116C in a state in which the ratchet member 5543 is spaced away from the reciprocating member 5551.

As a result, the operation member 5540 can be moved backward, i.e., can be moved toward the proximal end 52105 by the retraction wire 5309.

As described above, the present disclosure has been described with reference to the embodiment described with reference to the drawings, but it will be understood that this is merely exemplary, and those of ordinary skill in the art will understand that various modifications and other equivalent embodiments are possible therefrom. Accordingly, the true technical protection scope of the present disclosure should be defined by the technical spirit of the appended claims.

The embodiments may be represented by functional block elements and various processing steps. The functional blocks may be implemented as a varying number of hardware and/or software components that perform particular functions. For example, the embodiments may adopt integrated circuit configurations, such as memory, processing, logic, and/or look-up table, which may execute various functions by the control of one or more microprocessors or other control devices.

The particular implementations shown and described herein are illustrative examples of the embodiments and are not intended to otherwise limit the scope of the embodiments in any way. For the sake of brevity, conventional electronics, control methods, software development and other functional aspects of the methods may not be described in detail. Further, the connecting lines or connectors shown in the drawings are intended to represent example functional relationships and/or physical or logical couplings between the various elements. It should be noted that many alternative or additional functional relationships, physical connections, or logical connections may be present in a practical device. In addition, no item or component is essential to the practice of the present disclosure unless the component is specifically described as “essential” or “critical”.

The use of the terms “a” and “an” and “the” and similar referents in the context of describing the present disclosure (especially in the context of the following claims) are to be construed to cover both the singular and the plural. Further, recitation of ranges of values herein are merely intended to serve as a shorthand method of referring individually to each separate value falling within the range, unless otherwise indicated herein, and each separate value is incorporated into the specification as if it were individually recited herein. Finally, operations of all methods described herein can be performed in any suitable order unless otherwise indicated herein or otherwise clearly contradicted by context. The present disclosure is not necessarily limited to the described order of the operations. The use of any and all examples, or exemplary terms (e.g., “such as”) provided herein, is intended merely to better illuminate the present disclosure and does not pose a limitation on the scope of the present disclosure unless otherwise claimed. Further, numerous modifications and adaptations will be readily apparent to one of ordinary skill in the art without departing from the spirit and scope of the present disclosure.

1. A surgical instrument comprising:
an end tool that includes a jaw having a first jaw and a second jaw formed to face the first jaw, a first jaw pulley coupled to the jaw and formed to be rotatable around a first shaft, an opposing pulley formed to be rotatable around a shaft substantially the same as or parallel to the first shaft and formed to be spaced apart from the first jaw pulley by a certain extent, and a staple drive assembly including one or more staple pulleys at least partially formed between the first jaw pulley or the opposing pulley;
a cartridge that includes a reciprocating assembly connected to the staple drive assembly and configured to move linearly in response to a rotational movement of the one or more staple pulleys, and an operation member that is in contact with the reciprocating assembly and formed to move in one direction by the reciprocating assembly when the reciprocating assembly moves in the one direction; and
a retraction wire connected to the operation member and formed to pull the operation member toward a proximal end of the jaw.

2. The surgical instrument of claim 1, wherein the opposing pulley is a pulley for the retraction wire, with which the retraction wire comes into contact.

3. The surgical instrument of claim 1, wherein the opposing pulley is a second jaw pulley coupled to the second jaw.

4. The surgical instrument of claim 1, wherein, when the one or more staple pulleys rotate, the reciprocating assembly connected to the staple drive assembly moves toward a distal end or a proximal end of the cartridge.

5. The surgical instrument of claim 4, wherein, when the one or more staple pulleys alternately rotate in a clockwise direction and a counterclockwise direction, the reciprocating assembly connected to the staple drive assembly alternately moves toward the distal end and the proximal end of the cartridge.

6. The surgical instrument of claim 5, wherein, when the reciprocating assembly moves toward the distal end of the cartridge, the operation member moves toward the distal end of the cartridge by the reciprocating assembly.

7. The surgical instrument of claim 1, wherein the staple drive assembly includes a first staple pulley and a second staple pulley that face each other and rotationally move.

8. The surgical instrument of claim 7, wherein a bidirectional rotational movement of the first staple pulley or the second staple pulley is converted into a reciprocating linear movement of the reciprocating assembly connected to the staple drive assembly, by the staple drive assembly.

9. The surgical instrument of claim 7, wherein, when the first staple pulley rotates in a first direction among a clockwise direction and a counterclockwise direction, and the second staple pulley rotates in a direction opposite to the first direction among the clockwise and counterclockwise directions, the reciprocating assembly connected to the first and second staple pulleys and the operation member in contact with the reciprocating assembly move toward a distal end of the cartridge.

10. The surgical instrument of claim 9, wherein, when the first staple pulley rotates in the direction opposite to the first direction among the clockwise and counterclockwise directions, and the second staple pulley rotates in the first direction among the clockwise and counterclockwise directions, the reciprocating assembly connected to the one or more staple pulleys moves toward a proximal end of the end tool, and the operation member remains stationary with respect to the one direction.

11. The surgical instrument of claim 1, wherein,
as the operation member moves in the one direction,
a wedge of the operation member sequentially pushes and raises a plurality of staples in the cartridge to perform a stapling motion, and simultaneously,
a blade formed on one side of the wedge of the operation member moves in the one direction to perform a cutting motion.

12. The surgical instrument of claim 1, wherein the staple drive assembly includes a link member configured to connect the one or more staple pulleys to the reciprocating assembly.

13. The surgical instrument of claim 12, wherein
the operation member includes a ratchet member having at least one surface on which a ratchet is formed, and
the ratchet of the ratchet member is formed to be in contact with the reciprocating assembly.

14. The surgical instrument of claim 13, wherein the operation member moves toward a distal end of the cartridge together with the reciprocating assembly only when the reciprocating assembly moves toward the distal end of the cartridge.

15. The surgical instrument of claim 13, wherein the ratchet member of the operation member and the reciprocating assembly are separated from each other by the retraction wire.

16. The surgical instrument of claim 15, wherein the operation member is formed to be movable toward a proximal end of the end tool by the retraction wire while being spaced apart from the reciprocating assembly.

17. The surgical instrument of claim 15, further comprising an operation part elastic member disposed on one side of the operation member and configured to apply an elastic force in a direction that brings the ratchet member of the operation member and the reciprocating assembly closer to each other.

18. The surgical instrument of claim 17, wherein, when the retraction wire is pulled, the ratchet member presses the operation part elastic member in a direction in which the operation part elastic member moves away from the reciprocating assembly.

19. The surgical instrument of claim 1, wherein
an elastic member is disposed between the second jaw and the first jaw, and
in a standby state, the second jaw and the first jaw maintain an open state through the elastic member.

20. The surgical instrument of claim 19, wherein the elastic member is disposed to apply an elastic force in a direction in which the second jaw moves away from the first jaw.

21. The surgical instrument of claim 19, wherein, by moving the operation member, one region of the operation member is disposed inside the second jaw, thereby applying a force that reduces a gap between the second jaw and the first jaw and maintains a closed state of the second jaw and the first jaw.

22. The surgical instrument of claim 1, wherein the second jaw is coupled to the first jaw, and is formed to rotationally move integrally with the first jaw around the first shaft when the first jaw rotates.

23. The surgical instrument of claim 1, wherein
a passageway having a groove shape is formed in the second jaw in a longitudinal direction of the second jaw, and
a clamp of the operation member is formed to be movable in the passageway.

24. The surgical instrument of claim 23, wherein a guide part with an opening is formed in one region of the passageway at a proximal end side to allow the clamp of the operation member to be inserted into the second jaw.

25. The surgical instrument of claim 24, wherein, when the operation member moves from a proximal end toward a distal end of the end tool, one region of the clamp of the operation member passes through the guide part of the second jaw and moves along the passageway of the second jaw, thereby maintaining a closed state of the second jaw and the first jaw.

26. The surgical instrument of claim 1, wherein the operation member is formed to move toward a proximal end of the end tool independently of the reciprocating assembly by pulling the retraction wire.

27. The surgical instrument of claim 1, wherein the retraction wire and the operation member are integrally coupled to each other.

28. The surgical instrument of claim 1, wherein the retraction wire is formed to be attachable and detachable by being fastened to or unfastened from the operation member.

29. The surgical instrument of claim 1, wherein a backward-movement prevention member is formed on one side of the operation member to restrict a backward movement of the operation member caused by the reciprocating assembly when the reciprocating assembly moves backward in a proximal end direction.

30. The surgical instrument of claim 29, wherein the backward-movement prevention member of the operation member engages with an engaging part on an inner surface of a space of the end tool, in which the operation member is disposed, to restrict the backward movement of the operation member.

31. The surgical instrument of claim 30, wherein the engaging part is formed on an inner side surface of the first jaw and includes one or more protruding regions and one or more grooves.

32. The surgical instrument of claim 29, wherein the backward-movement prevention member is formed to perform a rotational movement.

33. The surgical instrument of claim 32, wherein
pulling a backward-movement prevention release wire connected to the backward-movement prevention member causes the backward-movement prevention member to perform the rotational movement, and
the restriction on the backward movement of the operation member by the backward-movement prevention member is released through the rotational movement of the backward-movement prevention member.

34. The surgical instrument of claim 1, further comprising one or more staple wires coupled to the one or more staple pulleys and configured to rotate the one or more staple pulleys.

35. The surgical instrument of claim 1, further comprising a pair of end tool first jaw pitch main pulleys formed to be rotatable around a second shaft that forms a certain angle with the first shaft.

36. The surgical instrument of claim 35, wherein the end tool is formed to be yaw-rotatable around the first shaft and simultaneously pitch-rotatable around the second shaft.

37. The surgical instrument of claim 1, wherein the first jaw pulley, the one or more staple pulleys, and the opposing pulley are sequentially stacked.
